(19) United States
(12) Patent Application Publication
(10) Pub. No.: US 2023/0227447 A1
(43) Pub. Date: Jul. 20, 2023

(54) BENZYLAMINE DERIVATIVES AS DDRS INHIBITORS

(71) Applicant: CHIESI FARMACEUTICI S.P.A., Parma (IT)

(72) Inventors: Laura CARZANIGA, Parma (IT); Fabio RANCATI, Parma (IT); Andrea RIZZI, Parma (IT); Anna KARAWAJCZYK, Parma (IT); Bartosz Pawel GUT, Parma (IT)

(73) Assignee: CHIESI FARMACEUTICI S.P.A., Parma (IT)

(21) Appl. No.: 17/927,762

(22) PCT Filed: May 24, 2021
(86) PCT No.: PCT/EP2021/063738

§ 371 Date: Nov. 25, 2022

(30) Foreign Application Priority Data
May 25, 2020 (EP) 20176360.4

(51) Int. Cl. C07D 471/04; C07D 487/04; C07D 417/12; C07D 403/12; C07D 401/12; C07D 239/42; C07D 213/81; C07D 239/26; C07D 519/00; C07D 213/74; C07D 401/04

(52) U.S. Cl. CPC C07D 471/04; C07D 487/04; C07D 417/12; C07D 403/12; C07D 401/12; C07D 239/42; C07D 213/81; C07D 239/26; C07D 519/00; C07D 213/74; C07D 401/04

(57) ABSTRACT

The present invention relates to a compounds of general formula (I) inhibiting DDR1 and DDR2, particularly the invention relates to compounds that are benzylamine derivatives, methods of preparing such compounds, pharmaceutical compositions containing them and therapeutic use thereof. The compounds of the invention may be useful in the treatment of diseases or conditions associated with a dysregulation of DDRs, in particular fibrosis.

### FIELD OF THE INVENTION

The present invention generally relates to compounds inhibiting Discoidin Domain Receptors (hereinafter DDR inhibitors); the invention relates to compounds that are benzylamine derivatives, methods of preparing such compounds, pharmaceutical compositions containing them and therapeutic use thereof.

The compounds of the invention may be useful for instance in the treatment of many disorders associated with DDR mechanisms.

### BACKGROUND OF THE INVENTION

The discoidin domain receptor (DDR) family comprises two distinct members, DDR1 and DDR2. DDRs are type I transmembrane receptor tyrosine kinase (RTKs), that display an overall structural organization that is similar to many members of the RTK family. They were initially discovered in the early 1990s by homology cloning based on their catalytic kinase domains (KD) (see Johnson, J. D. (1993) Proc. Natl. Acad. Sci. U.S.A. 90, 5677-5681; Di Marco, E. (1993) J. Biol. Chem. 268, 24290-24295; Zerlin, M. (1993) Oncogene 8, 2731-2739; Perez, J. L. (1996) Oncogene 12, 1469-1477).

Subsequently, collagens were identified as ligands for DDRs (see Vogel, W. (1997) Mol. Cell 1, 13-23; Shrivastava A. Mol Cell. 1997; 1:25-34.), thus establishing the unique characteristic of these receptors among other members of the RTK superfamily, that are typically activated by soluble peptide-like growth factors.

All DDRs are single-pass type I transmembrane glycoproteins that are characterized by the presence of six distinct domains: a discoidin (DS) domain, a DS-like domain, an extracellular juxtamembrane (EJXM) region, a transmembrane (TM) segment, a long intracellular juxtamembrane (IJXM) region, and an intracellular kinase domain (KD). The presence of the N-terminal DS and DS-like domains is the defining feature of the DDR RTK subfamily.

The DS domain contains the collagen-binding region and is responsible for mediating DDR specificity for fibrillar and non-fibrillar collagens (see Curat, C. A. (2001) J. Biol. Chem. 276, 45952-45958; Leitinger, B. (2003) J. Biol. Chem. 278, 16761-16769; Abdulhussein, R. (2004) J. Biol. Chem. 279, 31462-31470; Xu, H. (2011) Matrix Biol. 30, 16-26). The function of the DS-like domain of DDRs is not fully understood, but published data suggest that it contributes to collagen-induced receptor activation (see Carafoli, F. (2012) Structure 20, 688-697).

The EJXM region of human DDRs (49 residues in DDR1 and 31 residues in DDR2), which connects the DS domain to the TM segment, is of unknown structure. The EJXM region contains several putative N- and O-glycosylation sites, which may regulate receptor trafficking, turnover, and/or ligand-induced activation (see Curat, C. (2001) J. Biol. Chem. 276, 45952-45958).

A short TM helical segment (˜20 residues) links the ectodomain and the intracellular domains of DDRs. The TM segment plays a role in receptor dimerization (see Noordeen, N. A. (2006) J. Biol. Chem. 281, 22744-22751).

An unusually large (130-140 residues) IJXM region connects the TM segment with the KD. The IJXM region contains several tyrosine residues that serve as docking sites for cytoplasmic effectors and regulators that are essential for signal transduction. A classical KD (˜300 residues) follows the IJXM region in both DDR1 and DDR2.

The DDR1 subfamily is composed of five membrane-anchored isoforms, and the DDR2 subfamily is represented by a single protein. The five DDR1 isoforms are generated by alternative splicing. They all have in common the extracellular and transmembrane domains but differ in the cytoplasmic region. Of the five DDR1 isoforms, three (DDR1a, DDR1b, DDR1c) are functional receptors (see Valiathan, R. R. (2012) Cancer Metastasis Rev. 31, 295-321; Alves, F. (2001) FASEB J. 15, 1321-1323).

DDRs are unique among RTKs because they are activated by an extracellular matrix protein, collagen. The DDRs only bind collagen in its native, triple-helical conformation and do not recognize heat denatured collagen (gelatin) (see Vogel, W. (1997) Mol. Cell 1, 13-23; Leitinger, B. (2003) J. Biol. Chem. 278, 16761-16769).

Both DDRs display broad collagen specificity and are activated by many different collagen types, with fibrillar collagens (I-III and V) acting as ligands for both receptors (see Vogel, W. (1997) Mol. Cell 1, 13-23; Shrivastava A. Mol Cell. 1997; 1:25-34). The DDRs have distinct preferences for certain types of collagens. DDR1, but not DDR2, binds to the basement membrane collagen IV, while DDR2 seems to preferentially bind collagen II and collagen X (see Leitinger B. J Mol Biol. 2004; 344(4):993-1003; Leitinger B. Matrix Biol. 2006; 25(6):355-364). Similar to collagen-binding integrins, the DDRs recognize specific amino acid motifs in collagen. Detailed studies, utilizing libraries of triple-helical peptides, uncovered a six amino acids motif, GVMGFO, as a binding motif for both DDRs (see Farndale RWet al. Biochem Soc Trans. 2008; 36 (Pt 2):241-250).

The DDRs are unusual RTKs in that they form ligand-independent stable dimers that are non-covalently linked (see Noordeen, N. A. (2006) J. Biol. Chem. 281, 22744-22751; Mihai C. J Mol Biol. 2009; 385:432-445). DDR dimers likely form during biosynthesis and exist on the cell surface prior to ligand binding. Upon collagen binding, DDRs undergo tyrosine autophosphorylation. The two distinguishing features of DDR phosphorylation dynamics are a delayed and a sustained response. While typical RTKs are activated within seconds to minutes, maximal DDR activation is often achieved only hours after stimulation with collagen and can remain detectable for up to several days post-stimulation (see Vogel, W. (1997) Mol. Cell 1, 13-23; Shrivastava A. Mol Cell. 1997; 1:25-34). The molecular basis and the biological effects of these two intriguing characteristics of DDR phosphorylation are poorly understood.

Phosphorylation of tyrosine residues within the intracellular domains of activated DDRs generates docking sites for SH2, SH3, and PTB domain-containing proteins (see Wang, C. Z, (2006) Mol. Biol. Cell 17, 2839-2852); Lemeer, S., (2012) J. Proteomics 75, 3465-3477; L'hôte, C. G. (2002) FASEB J. 16, 234-236; Koo, D. H. (2006) FEBS Lett. 580, 15-22; Yang, G., (2009) Proteomics 9, 4944-4961).

Evidence so far suggests that stimulation of DDR1 with collagen is coupled to the activation of the PI3K/Akt and Ras/ERK MAPK cascades (see Lu, K. (2011) Cardiovasc. Pathol. 20, 71-76; Suh, H. N., J. Cell. Phyisiol. 226, 3422-3432; Ongusaha, P. P., EMBO J. 22, 1289-1301).

In the case of DDR2, the evidence points to a role for Src as a downstream effector and regulator of DDR2 signaling (see Ikeda, K., (2002) J. Biol. Chem. 277, 19206-19212; Olaso, E. (2011) Fibrogenesis Tissue Repair 4, 5; Yang, K., J. Biol. Chem. 280, 39058-39066).

The importance of DDRs as collagen receptors is demonstrated by the phenotype of DDR knock-out mice. While both DDR1 and DDR2 knockout mice are viable, they are small in size compared to wild type littermates (see Vogel W F, Mol Cell Biol. 2001; 21(8):2906-2917; Labrador J P. EMBO Rep. 2001; 2(5):446-452). DDR1 knockout mice have poorly mineralized fibula bones. In DDR2 knockout mice, dwarfism has been linked to shorter long bones that arise due to reduced chondrocyte proliferation. In humans, DDR2 mutations are associated with multiple skeletal defects, including short limbs and abnormal calcification. Besides being smaller in size, DDR knockout/mutant mice exhibit defects in reproduction. DDR1 knockout mice are unable to lactate due to aberrant mammary gland morphogenesis. Additionally, DDR1 knockout mice exhibit altered kidney structure and impaired primary mesangial cell adhesion to ECM (see Gross O, Kidney Int. 2004; 66(1):102-111; Curat C A, J Am Soc Nephrol. 2002; 13(11):2648-2656). These mice are also unable to control their ear movements and show loss of auditory function with profound structural changes throughout the cochlear duct (see Meyer zum Gottesberge A M, Lab Invest. 2008; 88(1): 27-37). DDR2 knockout mice, in contrast, show no defects in lactation, kidney structure, or auditory function. Instead these mice display impaired dermal wound healing due to defective proliferation, invasion, proteolytic activity, and ECM remodeling by skin fibroblasts (see Olaso E, J Biol Chem. 2002; 277(5):3606-3613)

Despite some of the developmental defects found in DDR-null mice, these mice have been valuable in understating the role of these receptors in multiple diseases, including lung fibrosis.

The first evidence for a protective role of DDR1 deletion in lung fibrosis was generated in 2006 by the research group of Dr. Vogel (see Avivi-Green C, Am J Respir Crit Care Med 2006; 174:420-427). The authors demonstrated that DDR1-null mice were largely protected against bleomycin (BLM)-induced injury. Furthermore, myofibroblast expansion and apoptosis were much lower in these animals compared with their wild-type counterparts. Absence of inflammation in knockout mice was confirmed by lavage cell count and cytokines ELISA. These results indicated that DDR1 expression is a prerequisite for the development of lung inflammation and fibrosis.

The above results have been confirmed using a pharmacological approach (and therapeutic regimes) by Wang Z. et al. (see Wang, Z., J. Med. Chem. 2016, 59, 5911-5916). Mice were treated with compound 6j (a tetrahydroisoquinoline derivative) after the onset of BLM-induced fibrotic injury. Compound 6j prevented BLM-induced pathological changes (i.e., reduction in alveolar spaces and ECM deposition) in a dose-dependent manner. This histological result was accompanied by reduced expression levels of fibrotic markers fibronectin, α-SMA, and collagen.

The role of DDR2 in organ fibrosis is less-well understood and controversial. DDR2-null mice have increased liver fibrosis after chronic liver injury (see Olaso E, Am J Pathol 2011; 179:2894-2004). On the other hand, DDR2 deficiency or downregulation reduces bleomycin-induced lung fibrosis (see Zhao H, Bian H, Bu X, Zhang S, Zhang P, Yu J, et al Mol Ther 2016; 24:1734-1744). Zhao et al, demonstrated that DDR2 plays a critical role in the induction of fibrosis and angiogenesis in the lung. The authors showed that DDR2 synergizes with transforming growth factor (TGF)-β to induce myofibroblast differentiation. Furthermore, they showed that treatment of injured mice with specific siRNA against DDR2 exhibited therapeutic efficacy against lung fibrosis. In a second publication, Jia et al showed that mice lacking DDR2 are protected from bleomycin-induced lung fibrosis (see Jia S, Am J Respir Cell Mol Biol 2018; 59:295-305). The authors demonstrated that, after bleomycin treatment, DDR2-null mice present a markedly preserved alveolar structure, with airspaces clear of heavy cellular infiltrate. In addition, DDR2-null fibroblasts are significantly more prone to apoptosis than wild-type fibroblasts, supporting a paradigm in which fibroblast resistance to apoptosis is critical for progression of fibrosis.

Various compounds have been described in the literature as DDR1 or DDR2 antagonists.

WO2015004481 (Astex) discloses bicyclic compounds as DDR1 and DDR2 inhibitors useful in the treatment of diseases such as cancer.

WO2017005583 (F. Hoffmann-La Roche) discloses triazaspiro derivatives as DDR1 inhibitors, useful for the treatment of renal conditions, liver conditions, inflammatory conditions, vascular conditions, cardiovascular conditions, fibrotic diseases, cancer and acute and chronic organ transplant rejection.

WO2014032755 (Merck) discloses compounds useful for the treatment and/or prophylaxis of physiological and/or pathophysiological states in the triggering of which DDR2 is involved, in particular for use in the treatment and/or prophylaxis of osteoarthritis.

WO2013161851 (Chugai) discloses benzamide derivatives as DDR1 antagonists useful for the treatment of fibrosis and/or inflammation.

WO2015060373 (Chugai) discloses quinazolinone and isoquinolinone derivatives as DDR1 antagonist useful for the treatment of fibrosis and/or inflammation.

WO2016064970 (Guangzhou) discloses isoquinolines derivatives as DDR1 inhibitors useful as therapeutic agents for preventing and treating inflammation, liver fibrosis, kidney fibrosis, lung fibrosis, skin scar, atherosclerosis, and cancer.

WO2005092896 (Jeil Pharmaceutical) discloses furopyrimidine derivatives as DDR2 inhibitors useful in treating illnesses caused by the DDR2 tyrosine kinase activity such as hepatocirrhosis, rheumatoid arthritis or cancer.

WO2010062038 (Legochem) discloses compounds as DDR1 and DDR2 inhibitors useful for the treatment of diseases such as a cancer, hepatocirrhosis, arteriosclerosis, rheumatoid arthritis, osteoarthritis, which are known to be mainly caused by an excessive activation DDR1 and DDR2.

WO2017038870 (Toray) discloses urea derivatives as DDR1 inhibitors, useful for the treatment of diseases wherein DDR1 receptors are involved.

Daniel E. Jeffries et al., in “Discovery of VU6015929: A Selective Discoidin Domain Receptor 1/2 (DDR1/2) Inhibitor to Explore the Role of DDR1 in Antifibrotic Therapy”, Med. Chem. Lett. 2020, 11, 29-33, disclose a selective dual DDR1/2 inhibitor, 7e (VU6015929), and suggest DDR1 inhibition as an exciting target for antifibrotic therapy.

Of note, antagonizing the DDR receptors may be useful for the treatment of fibrosis and disease, disorder and conditions that result from fibrosis and even more antagonizing both receptors DDR1 and DDR2 may be particularly efficacious in the treatment of the above-mentioned disease, disorder and conditions.

Several efforts have been done in the past years to develop novel DDR1 and DDR2 receptor antagonists useful for the treatment of several disease and some of those compounds have shown efficacy also in humans.

Despite the above cited prior art, there remains a potential for developing inhibitors of both receptors DDR1 and DDR2 useful for the treatment of diseases or conditions associated with a dysregulation of DDR receptors, in particular fibrosis.

In this respect, the state of the art does not describe or suggest benzylamine derivatives of general formula (I) of the present invention having an antagonist activity on receptor DDR which represent a solution to the aforementioned need.

### SUMMARY OF THE INVENTION

In a first aspect the invention refers to a compound of formula (I)

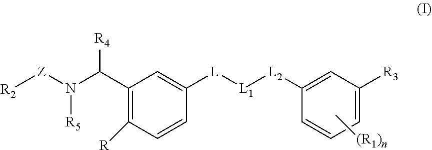

wherein
L and L₁ are different and independently selected from —C(O) and NH; L₂ is absent or NH, wherein when L and L₂ are both NH, L₁ is —C(O);
Z is absent or selected from —CH₂ and —C(O);
R₁ is H or selected from the group consisting of —O(C₁-C₄)alkyl,

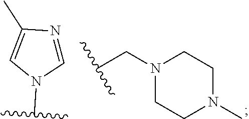

n is an integer from 1 to 3,
R is selected from the group consisting of (C₁-C₄)alkyl, halo, (C₁-C₄)haloalkyl and (C₃-C₆)cycloalkyl;
R₂ is selected from the group consisting of heteroaryl and heterocycloalkyl wherein each of said heteroaryl and heterocycloalkyl may be optionally substituted by one or more —C(O)NHR₆, —CN, (C₁-C₄)alkyl, halo, —NHC(O)R₆, heteroaryl and —NR₇R₈;
R₃ is selected from the group consisting of (C₁-C₄)alkyl, (C₁-C₄)haloalkyl, (C₃-C₆) cycloalkyl and —O(C₁-C₄)haloalkyl;
R₄ is H or selected from the group consisting of (C₁-C₄)alkyl, halo and (C₃-C₆) cycloalkyl;
R₅ is H or selected from the group consisting of (C₁-C₄)alkyl and heteroaryl(C₁-C₄)alkyl-;
R₆ is H or (C₁-C₄)alkyl;
R₇ and R₈ are at each occurrence independently H or selected from the group consisting of (C₁-C₄)alkyl, (C₃-C₈)cycloalkyl, (C₁-C₆)haloalkyl and halo; and pharmaceutically acceptable salts thereof.

In a second aspect, the invention refers to pharmaceutical composition comprising a compound of formula (I) in admixture with one or more pharmaceutically acceptable carrier or excipient.

In a third aspect, the invention refers to a compound of formula (I) for use as a medicament.

In a further aspect, the invention refers to a compound of formula (I) for use in treating disease, disorder, or condition associated with dysregulation of DDR.

In a further aspect, the invention refers to a compound of formula (I) for use in the prevention and/or treatment of fibrosis and/or diseases, disorders, or conditions that involve fibrosis.

In a further aspect, the invention refers to a compound of formula (I) for use in the prevention and/or treatment idiopathic pulmonary fibrosis (IPF).

In a further aspect, the invention refers to a compound of formula VIII

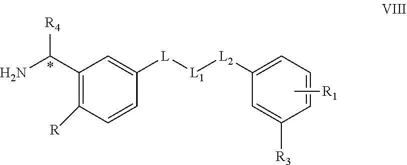

preferably for use as intermediate in the preparation of a series of compound of formula (I), wherein R, R₁, R₃, R₄, L, L₁ and L₂ are as indicated above for Formula (I).

In a further aspect, the invention refers to a compound of formula VII

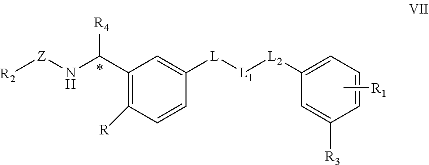

preferably for use as intermediate in the preparation of a series of compound of formula (I), wherein Z is absent, CH₂ or —C(O), R, R₁, R₂, R₃, R₄, L, L₁ and L₂ are as indicated above for Formula (I).

### DETAILED DESCRIPTION OF THE INVENTION

Unless otherwise specified, the compound of formula (I) of the present invention is intended to include also stereoisomer, tautomer or pharmaceutically acceptable salt or solvate thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUP AC Recommendations 1996, Pure and Applied Chemistry, 68:2193-2222 (1996)).

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “(Cₓ-C_{y}) alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The expressions “(Cₓ-C_{y}) haloalkyl” wherein x and y are integers, refer to the above defined “Cₓ-C_{y}alkyl” groups wherein one or more hydrogen atoms are replaced by one or more halogen atoms, which can be the same or different. Examples of said “(Cₓ-C_{y}) haloalkyl” groups may thus include halogenated, poly-halogenated and fully halogenated alkyl groups wherein all hydrogen atoms are replaced by halogen atoms, e.g. trifluoromethyl.

The term “aryl” refers to mono cyclic carbon ring systems wherein the ring is aromatic. Examples of suitable aryl monocyclic ring systems include, for instance, phenyl.

The term “heteroaryl” refers to a mono- or bi-cyclic aromatic ring system of 5 to 12 ring atoms containing one or more heteroatoms selected from S, N and O, and includes groups having two such monocyclic rings, or one such monocyclic ring and one monocyclic aryl ring, which are fused through a common bond. Examples for heteroaryl are pyridinyl, pyrimidinyl, imidazolyl, pyrazolyl, triazolyl, oxazolyl, oxadiazolyl, thiazolyl, thiadiazol, indazolyl, benzo[d][1,2,3]triazolyl, imidazo[1,5-a]pyridinyl, pyrazolo[3,4-b]pyridinyl, pyrazolo[4,3-b]pyridinyl, and tetrazolo[1,5-a]pyridinyl.

Particular examples for monocyclic heteroaryl are pyrimidinyl and pyridinyl.

Particular examples for bicycle heteroaryl are imidazo[1,2-a]pyridinyl, 1H-pyrrolo[2,3-b]pyridinyl, pyrazolo[1,5-a]pyrimidinyl, 1H-indazolyl, indazolyl, benzo[d]thiazolyl.

The term “heterocycloalkyl” refers to saturated or partly unsaturated mono or bicyclic ring system of 3 to 10 ring atoms comprising one or more heteroatoms selected from N, S or O. In particular embodiments, heterocycloalkyl is partly unsaturated bicyclic ring system of 7 to 9 ring atoms, comprising one or more heteroatoms selected from N, S or O. Particular example for bicyclic partly unsaturated heterocycloalkyl is 4,5,6,7-tetrahydropyrazolo[1,5-a]pyrimidinyl.

The term “(Cₓ-C_{y})cycloalkyl” wherein x and y are integers, refers to a monovalent saturated monocyclic or bicyclic hydrocarbon group of x to y ring carbon atoms. In particular embodiments, cycloalkyl refers to a monovalent saturated monocyclic hydrocarbon group of 3 to 8 ring carbon atoms. Bicyclic means consisting of two saturated carbocycles having one or more carbon atoms in common. Particular cycloalkyl groups are monocyclic. Examples for monocyclic cycloalkyl are cyclopropyl, cyclobutanyl, cyclopentyl, cyclohexyl or cycloheptyl.

The term “—O(Cₓ-C_{y})cycloalkyl” wherein x and y are integers, refers to the above defined “(Cₓ-C_{y})cycloalkyl” groups, wherein the carbon atom is linked to an oxygen atom. Examples include, e.g cyclopropyloxy.

The term “(Cₓ-C_{y}) aminoalkyl” wherein x and y are integers, refers to the above defined “(C₁-C₆) alkyl” groups wherein one or more hydrogen atoms are replaced by one or more amino group.

A bond pointing to a wavy or squiggly line, such as

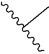

as used in structural formulas herein, depicts the bond that is the point of attachment of the moiety or substituent to the core or backbone structure.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—.

In general, the bracketed group is a lateral group, not included into the chain, and brackets are used, when deemed useful, to help disambiguating linear chemical formulas; e.g. the sulfonyl group —SO₂— might be also represented as —S(O)₂— to disambiguate e.g. with respect to the sulfinic group —S(O)O—.

Whenever basic amino or quaternary ammonium groups are present in the compounds of formula (I), physiologically acceptable anions may be present, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate. Likewise, in the presence of acidic groups such as COOH groups, corresponding physiological cation salts may be present as well, for instance including alkaline or alkaline earth metal ions.

The term “half maximal inhibitory concentration” (IC₅₀) indicates the concentration of a particular compound or molecule required for obtaining 50% inhibition of a biological process in vitro. IC₅₀ values can be converted logarithmically to pIC₅₀ values (−log IC₅₀), in which higher values indicate exponentially greater potency. The IC₅₀ value is not an absolute value but depends on experimental conditions e.g. concentrations employed. The IC₅₀ value can be converted to an absolute inhibition constant (Ki) using the Cheng-Prusoff equation (Biochem. Pharmacol. (1973) 22:3099).

As above indicated, the present invention refers to a series of compounds represented by the general formula (I) as herein below described in details, which are endowed with an inhibitory activity on receptors DDR₁ and DDR₂.

Advantageously, antagonizing both receptors DDR1 and DDR2 can be particularly efficacious in the treatment of those diseases where the DDR receptors play a relevant role in the pathogenesis such as fibrosis and disease, disorder and condition from fibrosis.

The compounds of formula (I) of the present invention are able to act as antagonist of both DDR1 and DDR2 receptors in a substantive and effective way, particularly appreciated by the skilled person when looking at a suitable and efficacious compounds useful for the treatment of fibrosis, in particular idiopathic pulmonary fibrosis.

As indicated in the experimental part, in fact, the compounds of formula (I) of the invention have activity on both receptors DDR1 and DDR2 as shown in Table 2, wherein for each compound is reported the potency expressed as inhibition constant (Ki).

As it can be appreciated, the compounds of the present invention according to Table 2, show a notable potency with respect to their inhibitory activity on both receptors DDR1 and DDR2 below about 1000 nM, even below 300 nM for most of the compounds confirming that they are able to antagonize the two isoforms of DDR receptor mainly involved in fibrosis and diseases that result from fibrosis.

In addition, some compounds of the invention are classified in Table 4 in term of potency (IC₅₀) with respect to their inhibitory activity against DDR1 and DDR2 receptors, according to the cell-based assay.

Therefore, the compounds of the present invention are particularly appreciated by the skilled person when looking at a suitable and efficacious compounds useful for the treatment of fibrosis, in particular idiopathic pulmonary fibrosis.

Thus, in one aspect the present invention relates to a compound of general formula (I) as DDR1 and DDR2 antagonist

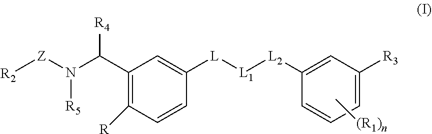

wherein
L and L₁ are different and independently selected from —C(O) and NH; L₂ is absent or NH, wherein when L and L₂ are both NH, L₁ is —C(O);
Z is absent or selected from —CH₂ and —C(O);
R₁ is H or selected from the group consisting of —O(C₁-C₄)alkyl,

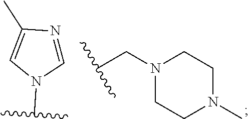

n is an integer from 1 to 3,
R is selected from the group consisting of (C₁-C₄)alkyl, halo, (C₁-C₄)haloalkyl and (C₃-C₆)cycloalkyl;
R₂ is selected from the group consisting of heteroaryl and heterocycloalkyl wherein each of said heteroaryl and heterocycloalkyl may be optionally substituted by one or more —C(O)NHR₆, —CN, (C₁-C₄)alkyl, halo, —NHC(O)R₆, heteroaryl and —NR₇R₈;
R₃ is selected from the group consisting of (C₁-C₄)alkyl, (C₁-C₄)haloalkyl, (C₃-C₆) cycloalkyl and —O(C₁-C₄)haloalkyl;
R₄ is H or selected from the group consisting of (C₁-C₄)alkyl, halo and (C₃-C₆) cycloalkyl;
R₅ is H or selected from the group consisting of (C₁-C₄)alkyl and heteroaryl(C₁-C₄)alkyl-;
R₆ is H or (C₁-C₄)alkyl;
R₇ and R₈ are at each occurrence independently H or selected from the group consisting of (C₁-C₄)alkyl, (C₃-C₈)cycloalkyl, (C₁-C₆)haloalkyl and halo; and pharmaceutically acceptable salts thereof.

In one preferred embodiment, the present invention refers to a compound of general formula (I)

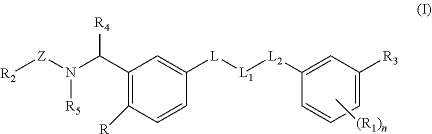

wherein
L and L₁ are different and independently selected from —C(O) and NH; L₂ is absent or NH;
Z is absent or selected from —CH₂ and —C(O);
R₁ is selected from the group consisting of —O(C₁-C₄)alkyl,

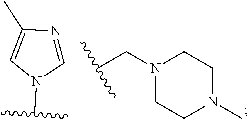

n is 1;
R is selected from the group consisting of (C₁-C₄)alkyl and halo;
R₂ is selected from the group consisting of heteroaryl and heterocycloalkyl wherein each of said heteroaryl and heterocycloalkyl may be optionally substituted by one or more —C(O)NHR₆ and CN;
R₃ is selected from the group consisting of (C₁-C₄)haloalkyl and —O(C₁-C₄)haloalkyl;

### R₄ is H;

R₅ is H or selected from the group consisting of (C₁-C₄)alkyl and heteroaryl(C₁-C₄)alkyl-;
R₆ is H or (C₁-C₄)alkyl;
and pharmaceutically acceptable salts thereof.

In a further preferred embodiment, the present invention refers to a compound of general formula (I) wherein R₁ is in meta with respect to the rest of the molecule, n is 1, L₂ is absent and R₄ is H, represented by the general formula (Ia)

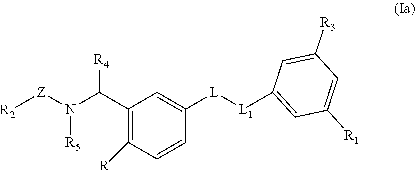

wherein
L and L₁ are different and independently selected from —C(O) and NH; Z is absent or selected from —CH₂ and —C(O);
R₁ is selected from the group consisting of —O(C₁-C₄)alkyl,

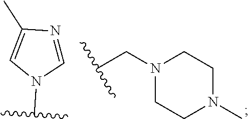

n is 1;
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-;
R₆ is H or (C₁-C₄)alkyl;
and pharmaceutically acceptable salts thereof.

In a further preferred embodiment, R₂ is selected from the group consisting of pyrimidinyl, pyridinyl, imidazo[1,2-a]pyridinyl, 1H-pyrrolo[2,3-b]pyridinyl, pyrazolo[1,5-a]pyrimidinyl, 1H-indazolyl, indazolyl, 4,5,6,7-tetrahydropyrazolo[1,5-a]pyrimidinyl and benzo[d]thiazolyl.

In a further preferred embodiment, the present invention refers to a compound of general formula (Ia), wherein L and L₁ are different and independently selected from —C(O) and NH;

Z is absent or selected from —CH₂ and —C(O);
R₁ is selected from the group consisting of —OCH₃,

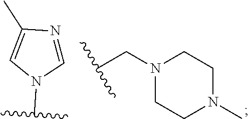

n is 1;
R is selected from the group consisting of methyl, ethyl, propyl and isopropyl;
R₂ is selected from the group consisting of pyrimidinyl, pyridinyl, imidazo[1,2-a]pyridinyl, 1H-pyrrolo[2,3-b]pyridinyl, pyrazolo[1,5-a]pyrimidinyl, 1H-indazolyl, indazolyl, 4,5,6,7-tetrahydropyrazolo[1,5-a]pyrimidinyl and benzo[d]thiazolyl wherein each of said heteroaryl and heterocycloalkyl may be optionally substituted by one or more —C(O)NHR₆ and CN;
R₃ is trifluoromethyl;
R₅ is H or selected from the group consisting of methyl, ethyl and 3-methylimidazo[1,2-a]pyridinyl;
R₆ is H or methyl;
and pharmaceutically acceptable salts thereof.

According to the preferred embodiment, the invention refers to at least one of the compounds listed in the Table 1 below; those compounds are active on receptors DDR1 and DDR2, as shown in Table 2.

TABLE 1
List of preferred compounds of Formula (I)

| Example | | |
|---|---|---|
| No. | Structure | Chemical Name |
| Example 22 | 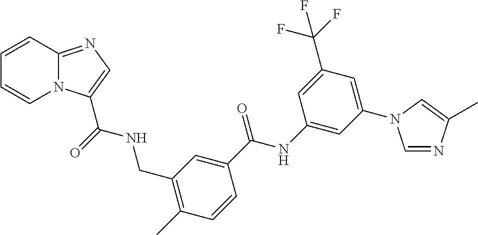 [SMILES: Cc1cn(-c2cc(NC(=O)c3ccc(C)c(CNC(=O)c4cnc5ccccn45)c3)cc(C(F)(F)F)c2)cn1] | N-(2-methyl-5-((3-(4- methyl-1H-imidazol-1-yl)- 5- (trifluoromethyl)phenyl) carbamoyl)benzyl)imidazo[1,2- a]pyridine-3-carboxamide |
| Example 19 | 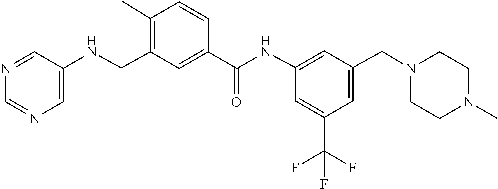 [SMILES: Cc1ccc(C(=O)Nc2cc(CN3CCN(C)CC3)cc(C(F)(F)F)c2)cc1CNc1cncnc1] | 4-methyl-N-(3-((4- methylpiperazin-1- yl)methyl)-5- (trifluoromethyl)phenyl)-3- ((pyrimidin-5- ylamino)methyl)benzamide |
| Example 23 | 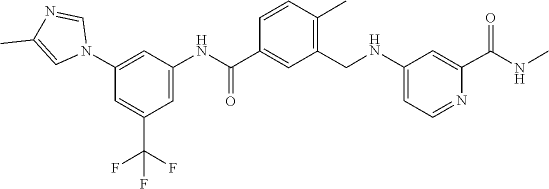 | N-methyl-4-((2-methyl-5- ((3-(4-methyl-1H-imidazol- 1-yl)-5- (trifluoromethyl)phenyl) carbamoyl)benzyl)amino) picolinamide |
| Example 24 | 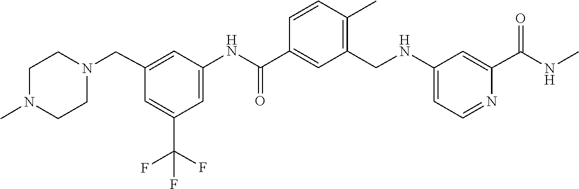 | N-methyl-4-((2-methyl-5- ((3-((4-methylpiperazin-1- yl)methyl)-5- (trifluoromethyl)phenyl) carbamoyl)benzyl)amino) picolinamide |
| Example 25 | 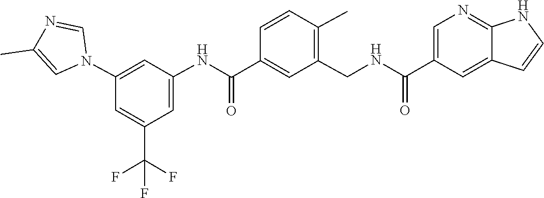 [SMILES: Cc1cn(-c2cc(NC(=O)c3ccc(C)c(CNC(=O)c4cnc5[nH]ccc5c4)c3)cc(C(F)(F)F)c2)cn1] | N-(2-methyl-5-((3-(4- methyl-1H-imidazol-1-yl)- 5- (trifluoromethyl)phenyl) carbamoyl)benzyl)-1H- pyrrolo[2,3-b]pyridine-5- carboxamide |
| Example 26 | 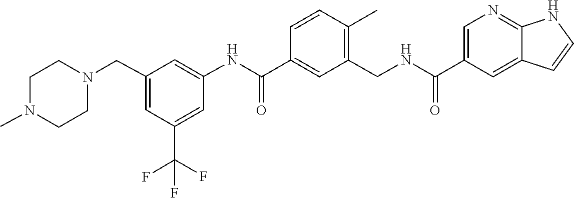 [SMILES: Cc1ccc(C(=O)Nc2cc(CN3CCN(C)CC3)cc(C(F)(F)F)c2)cc1CNC(=O)c1cnc2[nH]ccc2c1] | N-(2-methyl-5-((3-((4- methylpiperazin-1- yl)methyl)-5- (trifluoromethyl)phenyl) carbamoyl)benzyl)-1H- pyrrolo[2,3-b]pyridine-5- carboxamide |
| Example 21 | 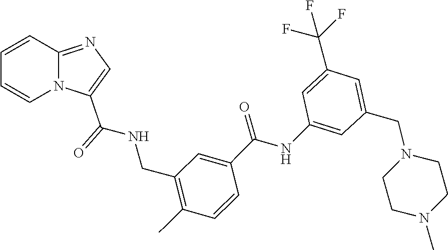 [SMILES: Cc1ccc(C(=O)Nc2cc(CN3CCN(C)CC3)cc(C(F)(F)F)c2)cc1CNC(=O)c1cnc2ccccn12] | N-(2-methyl-5-((3-((4- methylpiperazin-1- yl)methyl)-5- (trifluoromethyl)phenyl) carbamoyl)benzyl)imidazo[1,2- a]pyridine-3-carboxamide |
| Example 20 | 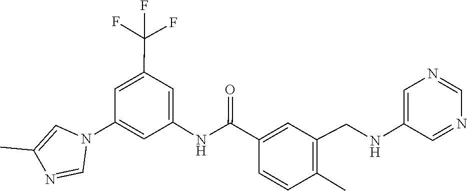 [SMILES: Cc1cn(-c2cc(NC(=O)c3ccc(C)c(CNc4cncnc4)c3)cc(C(F)(F)F)c2)cn1] | 4-methyl-N-(3-(4-methyl- 1H-imidazol-1-yl)-5- (trifluoromethyl)phenyl)-3- ((pyrimidin-5- ylamino)methyl)benzamide |
| Example 1 | 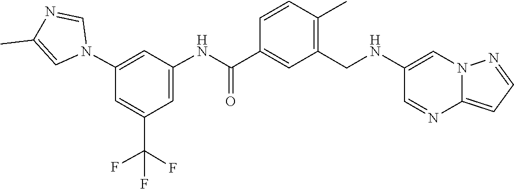 [SMILES: Cc1cn(-c2cc(NC(=O)c3ccc(C)c(CNc4cnc5ccnn5c4)c3)cc(C(F)(F)F)c2)cn1] | 4-methyl-N-(3-(4-methyl- 1H-imidazol-1-yl)-5- (trifluoromethyl)phenyl)-3- ((pyrazolo[1,5-a]pyrimidin- 6- ylamino)methyl)benzamide |
| Example 18 | 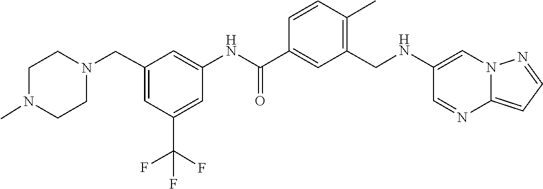 [SMILES: Cc1ccc(C(=O)Nc2cc(CN3CCN(C)CC3)cc(C(F)(F)F)c2)cc1CNc1cnc2ccnn2c1] | 4-methyl-N-(3-((4- methylpiperazin-1- yl)methyl)-5- (trifluoromethyl)phenyl)-3- ((pyrazolo[1,5-a]pyrimidin- 6- ylamino)methyl)benzamide |
| Example 28 | 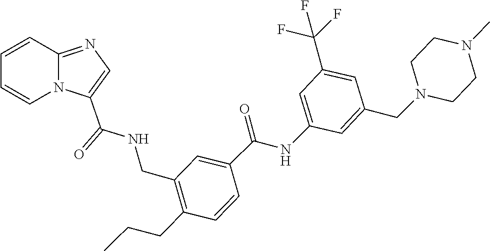 [SMILES: CCCc1ccc(C(=O)Nc2cc(CN3CCN(C)CC3)cc(C(F)(F)F)c2)cc1CNC(=O)c1cnc2ccccn12] | N-(5-((3-((4- methylpiperazin-1- yl)methyl)-5- (trifluoromethyl)phenyl) carbamoyl)-2- propylbenzyl)imidazo[1,2- a]pyridine-3-carboxamide formate salt |
| Example 30 | 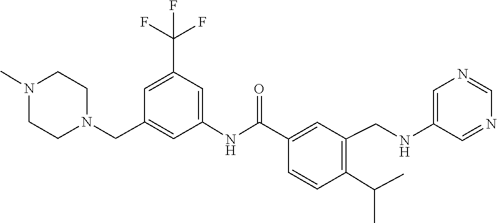 [SMILES: CC(C)c1ccc(C(=O)Nc2cc(CN3CCN(C)CC3)cc(C(F)(F)F)c2)cc1CNc1cncnc1] | 4-isopropyl-N-(3-((4- methylpiperazin-1- yl)methyl)-5- (trifluoromethyl)phenyl)-3- ((pyrimidin-5- ylamino)methyl)benzamide |
| Example 29 | 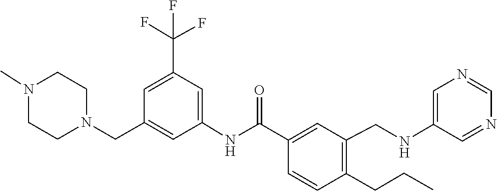 [SMILES: CCCc1ccc(C(=O)Nc2cc(CN3CCN(C)CC3)cc(C(F)(F)F)c2)cc1CNc1cncnc1] | N-(3-((4-methylpiperazin- 1-yl)methyl)-5- (trifluoromethyl)phenyl)-4- propyl-3-((pyrimidin-5- ylamino)methyl)benzamide |
| Example 10 | 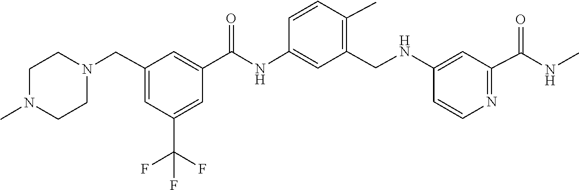 | N-methyl-4-((2-methyl-5- (3-((4-methylpiperazin-1- yl)methyl)-5- (trifluoromethyl)benzamido) benzyl)amino)picolinamide |
| Example 27 | 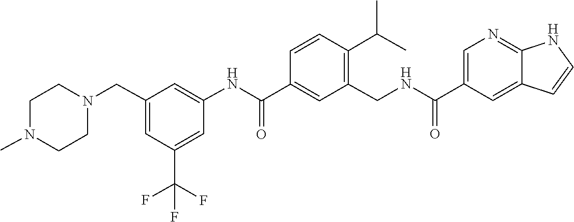 [SMILES: CC(C)c1ccc(C(=O)Nc2cc(CN3CCN(C)CC3)cc(C(F)(F)F)c2)cc1CNC(=O)c1cnc2[nH]ccc2c1] | N-(2-isopropyl-5-((3-((4- methylpiperazin-1- yl)methyl)-5- (trifluoromethyl)phenyl) carbamoyl)benzyl)-1H- pyrrolo[2,3-b]pyridine-5- carboxamide |
| Example 9 | 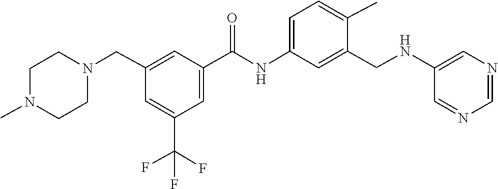 [SMILES: Cc1ccc(NC(=O)c2cc(CN3CCN(C)CC3)cc(C(F)(F)F)c2)cc1CNc1cncnc1] | N-(4-methyl-3-((pyrimidin- 5-ylamino)methyl)phenyl)- 3-((4-methylpiperazin-1- yl)methyl)-5- (trifluoromethyl)benzamide |
| Example 16 | 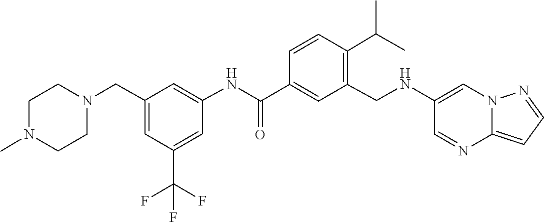 [SMILES: CC(C)c1ccc(C(=O)Nc2cc(CN3CCN(C)CC3)cc(C(F)(F)F)c2)cc1CNc1cnc2ccnn2c1] | 4-isopropyl-N-(3-((4- methylpiperazin-1- yl)methyl)-5 (trifluoromethyl)phenyl)-3- ((pyrazolo[1,5-a]pyrimidin- 6 ylamino)methyl)benzamide |
| Example 17 | 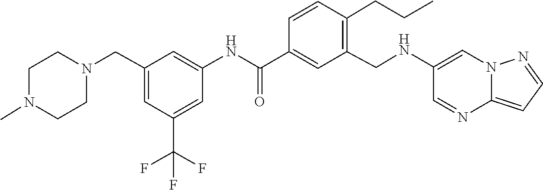 [SMILES: CCCc1ccc(C(=O)Nc2cc(CN3CCN(C)CC3)cc(C(F)(F)F)c2)cc1CNc1cnc2ccnn2c1] | N-(3-((4-methylpiperazin- 1-yl)methyl)-5- (trifluoromethyl)phenyl)-4- propyl-3-((pyrazolo[1,5- a]pyrimidin-6- ylamino)methyl)benzamide |
| Example 11 | 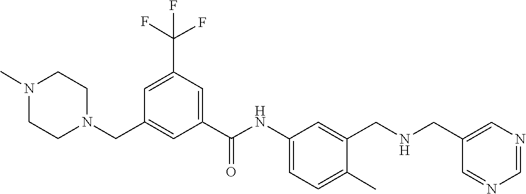 [SMILES: Cc1ccc(NC(=O)c2cc(CN3CCN(C)CC3)cc(C(F)(F)F)c2)cc1CNCc1cncnc1] | N-(4-methyl-3- (((pyrimidin-5- ylmethyl)amino)methyl) phenyl)-3-((4- methylpiperazin-1- yl)methyl)-5- (trifluoromethyl)benzamide |
| Example 12 | 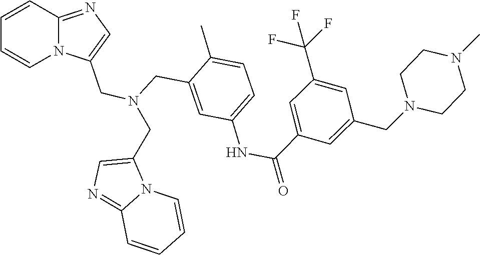 [SMILES: Cc1ccc(NC(=O)c2cc(CN3CCN(C)CC3)cc(C(F)(F)F)c2)cc1CN(Cc1cnc2ccccn12)Cc1cnc2ccccn12] | N-(3-((bis(imidazo[1,2- a]pyridin-3- ylmethyl)amino)methyl)-4- methylphenyl)-3-((4- methylpiperazin-1- yl)methyl)-5- (trifluoromethyl)benzamide |
| Example 14 | 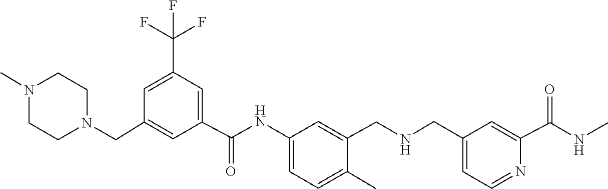 | N-methyl-4-(((2-methyl-5- (3-((4-methylpiperazin-1- yl)methyl)-5- (trifluoromethyl)benzamido) benzyl)amino)methyl) picolinamide |
| Example 15 | 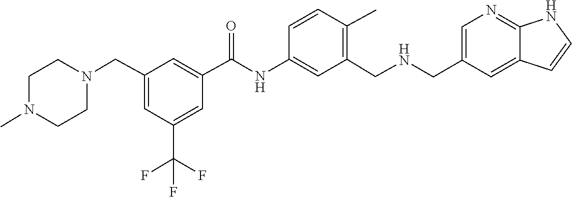 [SMILES: Cc1ccc(NC(=O)c2cc(CN3CCN(C)CC3)cc(C(F)(F)F)c2)cc1CNCc1cnc2[nH]ccc2c1] | N-(3-((((1H-pyrrolo[2,3- b]pyridin-5- yl)methyl)amino)methyl)- 4-methylphenyl)-3-((4- methylpiperazin-1- yl)methyl)-5- (trifluoromethyl)benzamide |
| Example 2 | 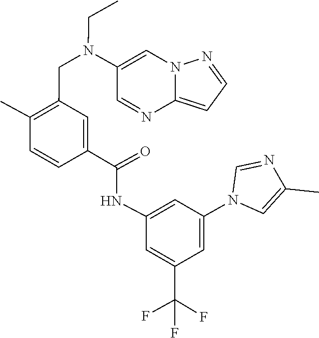 | 3-((ethyl(pyrazolo[1,5- a]pyrimidin-6- yl)amino)methyl)-4- methyl-N-(3-(4-methyl-1H- imidazol-1-yl)-5- (trifluoromethyl)phenyl) benzamide |
| Example 6 | 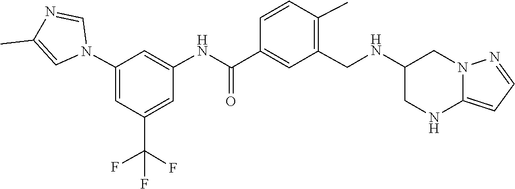 [SMILES: Cc1cn(-c2cc(NC(=O)c3ccc(C)c(CNC4CNc5ccnn5C4)c3)cc(C(F)(F)F)c2)cn1] | 4-methyl-N-(3-(4-methyl- 1H-imidazol-1-yl)-5- (trifluoromethyl)phenyl)-3- (((4,5,6,7- tetrahydropyrazolo[1,5- a]pyrimidin-6- yl)amino)methyl)benzamide |
| Example 5 | 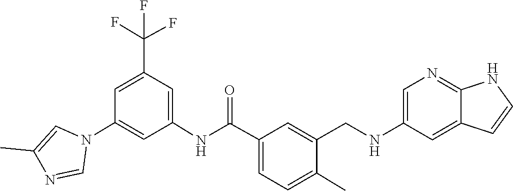 [SMILES: Cc1cn(-c2cc(NC(=O)c3ccc(C)c(CNc4cnc5[nH]ccc5c4)c3)cc(C(F)(F)F)c2)cn1] | 3-(((1H-pyrrolo[2,3- b]pyridin-5- yl)amino)methyl)-4- methyl-N-(3-(4-methyl-1H- imidazol-1-yl)-5- (trifluoromethyl)phenyl) benzamide |
| Example 4 | 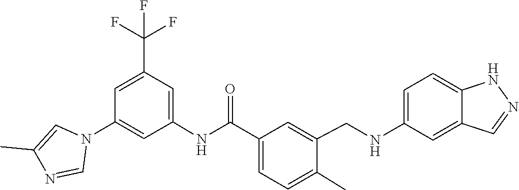 [SMILES: Cc1cn(-c2cc(NC(=O)c3ccc(C)c(CNc4ccc5[nH]ncc5c4)c3)cc(C(F)(F)F)c2)cn1] | 3-(((1H-indazol-5- yl)amino)methyl)-4- methyl-N-(3-(4-methyl-1H- imidazol-1-yl)-5- (trifluoromethyl)phenyl) benzamide |
| Example 8 | 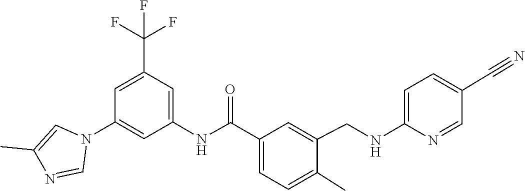 [SMILES: Cc1cn(-c2cc(NC(=O)c3ccc(C)c(CNc4ccc(C#N)cn4)c3)cc(C(F)(F)F)c2)cn1] | 3-(((5-cyanopyridin-2- yl)amino)methyl)-4- methyl-N-(3-(4-methyl-1H- imidazol-1-yl)-5- (trifluoromethyl)phenyl) benzamide |
| Example 7 | 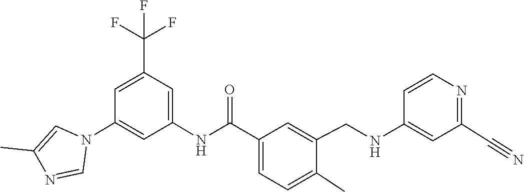 [SMILES: Cc1cn(-c2cc(NC(=O)c3ccc(C)c(CNc4ccnc(C#N)c4)c3)cc(C(F)(F)F)c2)cn1] | 3-(((2-cyanopyridin-4- yl)amino)methyl)-4- methyl-N-(3-(4-methyl-1H- imidazol-1-yl)-5- (trifluoromethyl)phenyl) benzamide |
| Example 3 | 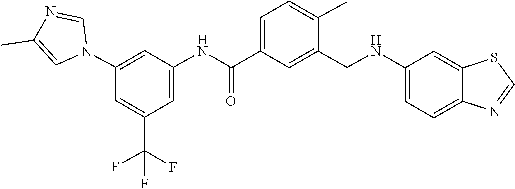 [SMILES: Cc1cn(-c2cc(NC(=O)c3ccc(C)c(CNc4ccc5ncsc5c4)c3)cc(C(F)(F)F)c2)cn1] | 3-((benzo[d]thiazol-6- ylamino)methyl)-4-methyl- N-(3-(4-methyl-1H- imidazol-1-yl)-5- (trifluoromethyl)phenyl) benzamide |
| Example 13 | 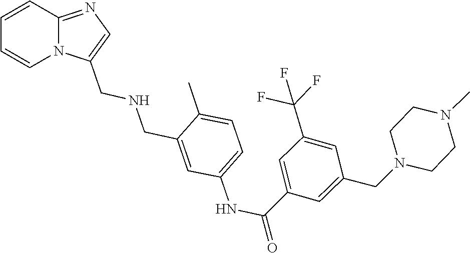 [SMILES: Cc1ccc(NC(=O)c2cc(CN3CCN(C)CC3)cc(C(F)(F)F)c2)cc1CNCc1cnc2ccccn12] | N-(3-(((imidazo[1,2- a]pyridin-3- ylmethyl)amino)methyl)-4- methylphenyl)-3-((4- methylpiperazin-1- yl)methyl)-5- (trifluoromethyl)benzamide |
| Example 31 | 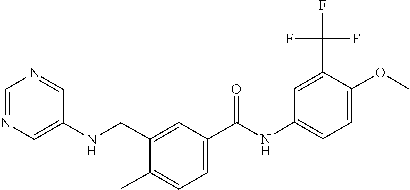 [SMILES: COc1ccc(NC(=O)c2ccc(C)c(CNc3cncnc3)c2)cc1C(F)(F)F] | N-(4-methoxy-3- (trifluoromethyl)phenyl)-4- methyl-3-((pyrimidin-5- ylamino)methyl)benzamide |
| Example 33 | 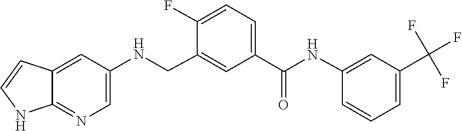 [SMILES: O=C(Nc1cccc(C(F)(F)F)c1)c1ccc(F)c(CNc2cnc3[nH]ccc3c2)c1] | 3-(((1H-pyrrolo[2,3-b] pyridin-5-yl)amino) methyl)-4-fluoro-N-(3 (trifluoromethyl)phenyl) benzamide |
| Example 32 | 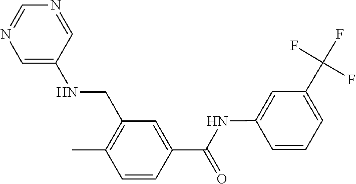 [SMILES: Cc1ccc(C(=O)Nc2cccc(C(F)(F)F)c2)cc1CNc1cncnc1] | 4-methyl-3-((pyrimidin-5- ylamino)methyl)-N-(3- (trifluoromethyl)phenyl) benzamide |
| Example 35 | 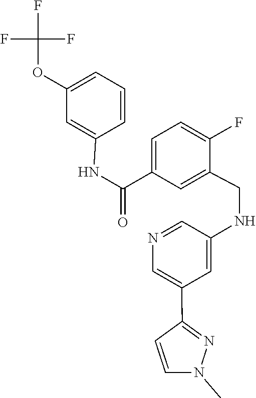 [SMILES: Cn1ccc(-c2cncc(NCc3cc(C(=O)Nc4cccc(OC(F)(F)F)c4)ccc3F)c2)n1] | 4-fluoro-3-(((5-(1-methyl- 1H-pyrazol-3-yl)pyridin-3- yl)amino)methyl)-N-(3- (trifluoromethoxy)phenyl) benzamide |
| Example 34 | 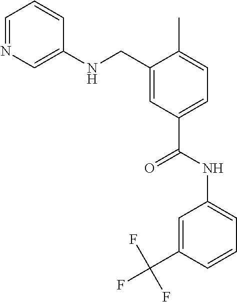 [SMILES: Cc1ccc(C(=O)Nc2cccc(C(F)(F)F)c2)cc1CNc1cccnc1] | 4-methyl-3-((pyridin-3- ylamino)methyl)-N-(3- (trifluoromethyl) phenyl)benzamide |
| Example 36 | 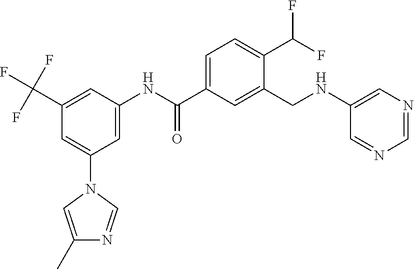 [SMILES: Cc1cn(-c2cc(NC(=O)c3ccc(C(F)F)c(CNc4cncnc4)c3)cc(C(F)(F)F)c2)cn1] | 4-(difluoromethyl)-N-(3- (4-methyl-1H-imidazol-1- yl)-5-(trifluoromethyl) phenyl)-3-((pyrimidin-5-yl amino)methyl)benzamide |
| Example 37 | 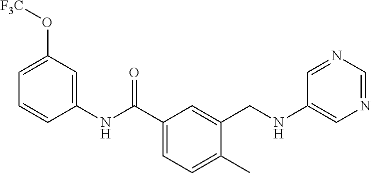 [SMILES: Cc1ccc(C(=O)Nc2cccc(OC(F)(F)F)c2)cc1CNc1cncnc1] | 4-methyl-3-((pyrimidin-5- ylamino)methyl)-N-(3- (trifluoromethoxy) phenyl)benzamide |
| Example 38 | 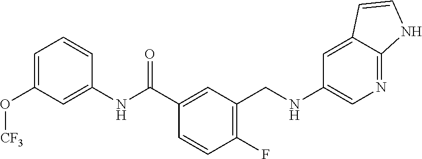 [SMILES: O=C(Nc1cccc(OC(F)(F)F)c1)c1ccc(F)c(CNc2cnc3[nH]ccc3c2)c1] | 3-(((1H-pyrrolo[2,3- b]pyridin-5- yl)amino)methyl)-4- fluoro-N-(3- (trifluoromethoxy)phenyl) benzamide |

In a further preferred embodiment, the invention refers to a compound of general formula (Ia) wherein R₁ is

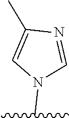

represented by the general formula (Ib)

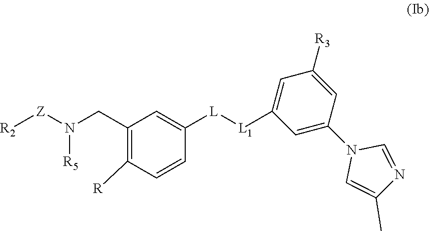

wherein
L and L₁ are different and independently selected from —C(O) and NH;
Z is absent or selected from —CH₂ and —C(O);
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-;
R₆ is H or (C₁-C₄)alkyl;
and pharmaceutically acceptable salts thereof.

In a further preferred embodiment, the invention refers to the compound of formula (Ib), wherein L and L₁ are different and independently selected from —C(O) and NH;

Z is absent or C(O);
R is methyl or propyl;
R₂ is selected from the group consisting of imidazo[1,2-a]pyridinyl, pyrimidinyl, pyridinyl, 1H-pyrrolo[2,3-b]pyridinyl, pyrazolo[1,5-a]pyrimidinyl, 1H-indazolyl, benzo[d]thiazolyl and 4,5,6,7-tetrahydropyrazolo[1,5-a]pyrimidinyl, wherein each of said heteroaryl and heterocycloalkyl may be optionally substituted by one or more —C(O)NHR₆ and CN;
R₃ is trifluoromethyl;
R₅ is H or ethyl;
R₆ is methyl.

In a further preferred embodiment, the invention refers to a compound of general formula (Ia) wherein R₁ is

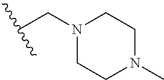

represented by the general formula (Ic)

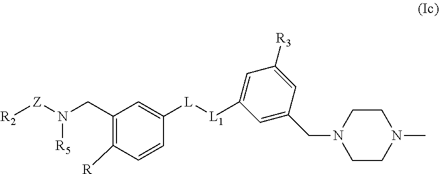

wherein
L and L₁ are different and independently selected from —C(O) and NH;
Z is absent or selected from —CH₂ and —C(O);
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-;
R₆ is H or (C₁-C₄)alkyl;
and pharmaceutically acceptable salts thereof.

In a further preferred embodiment, the invention refers to the compound of formula (Ic), wherein L and L₁ are different and independently selected from —C(O) and NH;

Z is absent or selected from —CH₂ and —C(O);
R is selected from the group consisting of methyl, propyl and isopropyl;
R₂ is selected from the group consisting of imidazo[1,2-a]pyridinyl, pyrimidinyl, pyridinyl 1H-pyrrolo[2,3-b]pyridinyl and pyrazolo[1,5-a]pyrimidinyl, wherein each of said heteroaryl and heterocycloalkyl may be optionally substituted by one or more —C(O)NHR₆;
R₃ is trifluoromethyl;
R₅ is H or 3-methylimidazo[1,2-a]pyridinyl;
R₆ is methyl;
and pharmaceutically acceptable salts thereof.

In a further embodiment, the invention refers to a compound of general formula (I) wherein L₂ is absent, n is 1, R₁ is —O(C₁-C₄)alkyl and is in para with respect to L₁, and R₄ is H, represented by the general formula (Id)

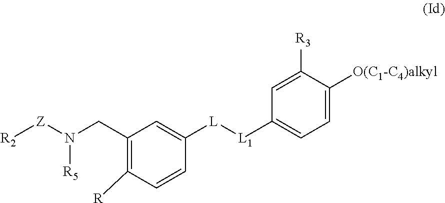

wherein
L and L₁ are different and independently selected from —C(O) and NH;
Z is absent or selected from —CH₂ and —C(O);
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-;
R₆ is H or (C₁-C₄)alkyl;
and pharmaceutically acceptable salts thereof.

In a further embodiment, the invention refers to the compound of formula (Id), wherein L and L₁ are different and independently selected from —C(O) and NH;

Z is absent or selected from —CH₂ and —C(O);
R is selected from the group consisting of methyl, propyl and isopropyl;
R₂ is selected from the group consisting of imidazo[1,2-a]pyridinyl, pyrimidinyl, pyridinyl 1H-pyrrolo[2,3-b]pyridinyl and pyrazolo[1,5-a]pyrimidinyl, wherein each of said heteroaryl and heterocycloalkyl may be optionally substituted by one or more —C(O)NHR₆;
R₃ is trifluoromethyl;
R₅ is H or 3-methylimidazo[1,2-a]pyridinyl;
R₆ is methyl;
and pharmaceutically acceptable salts thereof.

In a further embodiment, the invention refers to a compound of general formula (Id) wherein L₂ is absent, n is 1, R₁ is —OCH₃ and is in para with respect to L₁, and R₄ is H, represented by the general formula (Ie)

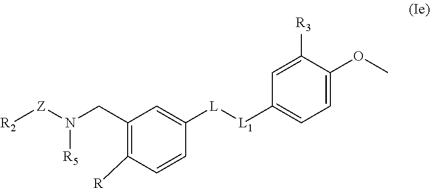

wherein
L and L₁ are different and independently selected from —C(O) and NH;
Z is absent or selected from —CH₂ and —C(O);
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-;
R₆ is H or (C₁-C₄)alkyl;
and pharmaceutically acceptable salts thereof.

In another preferred embodiment, the invention refers to the compound of formula (Ie), wherein L and L₁ are different and independently selected from —C(O) and NH;

Z is absent or selected from —CH₂ and —C(O);
R is selected from the group consisting of methyl, propyl and isopropyl;
R₂ is selected from the group consisting of imidazo[1,2-a]pyridinyl, pyrimidinyl, pyridinyl 1H-pyrrolo[2,3-b]pyridinyl and pyrazolo[1,5-a]pyrimidinyl, wherein each of said heteroaryl and heterocycloalkyl may be optionally substituted by one or more —C(O)NHR₆;
R₃ is trifluoromethyl;
R₅ is H or 3-methylimidazo[1,2-a]pyridinyl;
R₆ is methyl;
and pharmaceutically acceptable salts thereof.

In a further preferred embodiment, the invention refers to a compound of general formula (I) wherein L₂ is absent, R₄ and R₅ are —H, Z is absent, represented by the general formula (If)

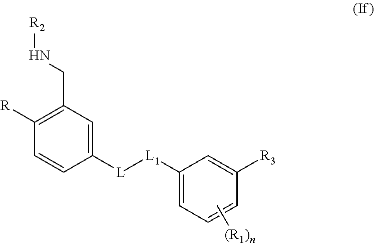

wherein

### L is —C(O); L₁ is —NH;

R₁ is H or selected from the group consisting of —O(C₁-C₄)alkyl and

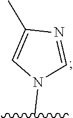 [SMILES: *n1cnc(C)c1]

R is selected from the group consisting of (C₁-C₄)alkyl and halo;
R₂ is selected from the group consisting of

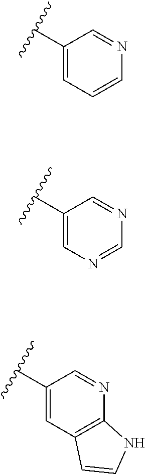 [SMILES: *c1cccnc1 ; *c1cncnc1 ; *c1cnc2[nH]ccc2c1]

R₃ is selected from the group consisting of (C₁-C₄)haloalkyl and —O(C₁-C₄)haloalkyl;
and pharmaceutically acceptable salts thereof.

In a further embodiment, the invention refers to a compound of general formula (If) wherein L is —C(O); L₁ is —NH;

R₁ is H or selected from the group consisting of —OCH₃ and

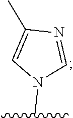 [SMILES: *n1cnc(C)c1]

R is selected from the group consisting of methyl and fluorine;
R₂ is selected from the group consisting of

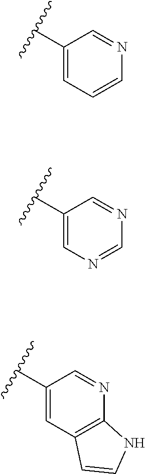 [SMILES: *c1cccnc1 ; *c1cncnc1 ; *c1cnc2[nH]ccc2c1]

R₃ is selected from the group consisting of trifluoromethyl and trifluoromethoxy;
and pharmaceutically acceptable salts thereof.

In an even further preferred embodiment, the invention refers to a compound of general formula (If) wherein L is —C(O), L₁ is —NH, R₁ is H or —OCH₃, R is selected from the group consisting of methyl and fluorine, R₂ is selected from the group consisting of

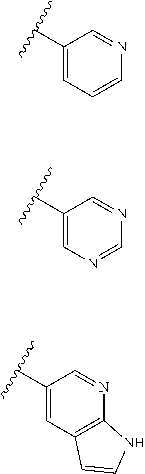

R₃ is trifluoromethyl;
and pharmaceutically acceptable salts thereof.

In a further preferred embodiment, the invention refers to at least one of the compounds listed in the Table 3 below. Those compounds are active on receptors DDR1 and DDR2, as shown in Tables 2 and 4.

TABLE 3
List of preferred compounds of Formula (If)

| Example | | |
|---|---|---|
| No. | Structure | Chemical Name |
| Example 20 | 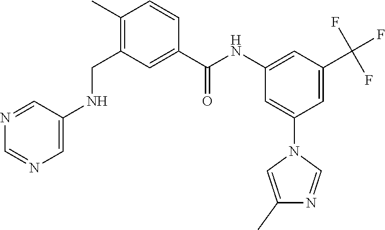 [SMILES: Cc1cn(-c2cc(NC(=O)c3ccc(C)c(CNc4cncnc4)c3)cc(C(F)(F)F)c2)cn1] | 4-methyl-N-(3-(4-methyl- 1H-imidazol-1-yl)-5- (trifluoromethyl)phenyl)-3- ((pyrimidin-5- ylamino)methyl)benzamide |
| Example 31 | 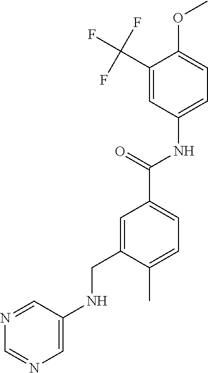 [SMILES: COc1ccc(NC(=O)c2ccc(C)c(CNc3cncnc3)c2)cc1C(F)(F)F] | N-(4-methoxy-3- (trifluoromethyl)phenyl)-4- methyl-3-((pyrimidin-5- ylamino)methyl)benzamide |
| Example 32 | 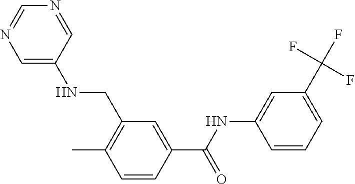 [SMILES: Cc1ccc(C(=O)Nc2cccc(C(F)(F)F)c2)cc1CNc1cncnc1] | 4-methyl-3-((pyrimidin-5- ylamino)methyl)-N-(3- (trifluoromethyl)phenyl) benzamide |
| Example 34 | 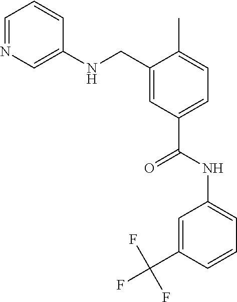 [SMILES: Cc1ccc(C(=O)Nc2cccc(C(F)(F)F)c2)cc1CNc1cccnc1] | 4-methyl-3-((pyridin-3- ylamino)methyl)-N-(3- (trifluoromethyl) phenyl)benzamide |
| Example 37 | 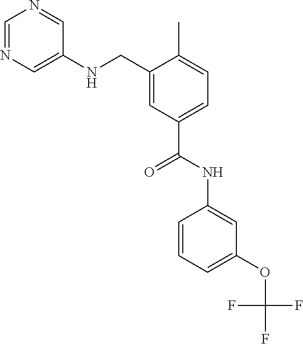 [SMILES: Cc1ccc(C(=O)Nc2cccc(OC(F)(F)F)c2)cc1CNc1cncnc1] | 4-methyl-3-((pyrimidin-5- ylamino)methyl)-N-(3- (trifluoromethoxy)phenyl) benzamide |
| Example 38 | 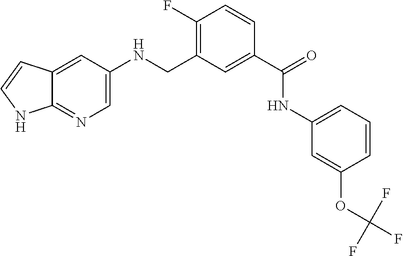 [SMILES: O=C(Nc1cccc(OC(F)(F)F)c1)c1ccc(F)c(CNc2cnc3[nH]ccc3c2)c1] | 3-(((1H-pyrrolo[2,3- b]pyridin-5- yl)amino)methyl)-4- fluoro-N-(3- (trifluoromethoxy)phenyl) benzamide |
| Example 33 | 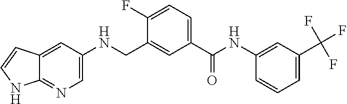 [SMILES: O=C(Nc1cccc(C(F)(F)F)c1)c1ccc(F)c(CNc2cnc3[nH]ccc3c2)c1] | 3-(((1H-pyrrolo[2,3-b] pyridin-5-yl)amino) methyl)-4-fluoro-N-(3 (trifluoromethyl)phenyl) benzamide |

The compounds of the invention, including all the compounds here above listed, can be prepared from readily available starting materials using the following general methods and procedures or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures.

Thus, processes described below and reported in the following schemes should not be viewed as limiting the scope of the synthetic methods available for the preparation of the compounds of the invention.

In some cases, generally known protective groups (PG) could be employed when needed to mask or protect sensitive or reactive moieties, in accordance to general principles of chemistry (Protective group in organic syntheses, 3rd ed. T. W. Greene, P. G. M. Wuts).

Processes of preparation described below and reported in the following Schemes should not be viewed as limiting the scope of the synthetic methods available for the preparation of the compounds of the invention.

The compounds of formula (I), including all the compounds here above listed, can be generally prepared according to the procedures shown in the schemes below. Where a specific synthetic step differs from what is described in the general schemes, it has been detailed in the specific examples, and/or in additional schemes.

Compounds of formula (I) contain at least one stereogenic center, as marked by an asterisk * in the picture below.

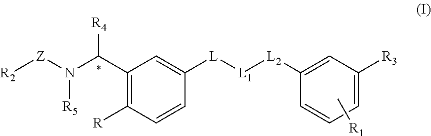

Enantiomerically pure compounds can be prepared from the corresponding racemates by means of chiral chromatography. Whenever, in compounds of formula (I), there are two or more stereogenic centers, the structure is then characterized by different stereoisomers. Stereochemically pure compounds may be obtained by chiral separation from a diastereoisomeric mixture, or stepwise by chromatographic separation of diastereoisomers followed by further chiral separation into single stereoisomers.

Compounds of formula (I) may be prepared according to SCHEME 1 as described hereinafter providing at least one non-limiting synthetic route for the preparation of all examples.

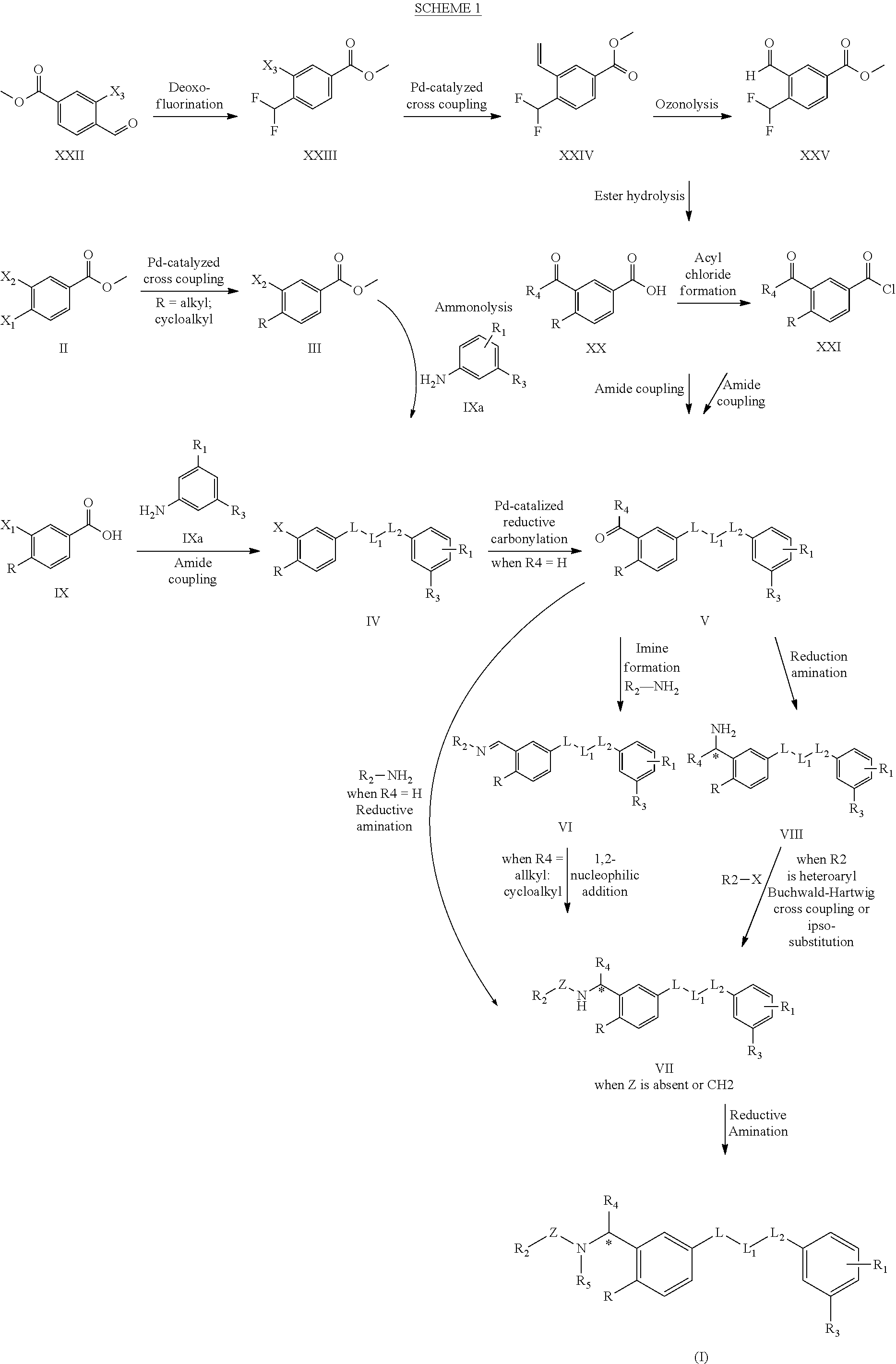

According to SCHEME 1, Intermediate III may be obtained from Intermediate II through a palladium catalyzed cross coupling on the most reactive leaving group between X₁ and X₂, wherein X₁ and X₂ can be, for example, chloride, bromide, iodide, OMs or OTs. For example the reaction may be carried out by reacting a bis-halide aryl intermediate II with an alkylboronic acid or potassium alkyltrifluoroborate following the classical Suzuki protocol, in a suitable s organic solvent such as Dioxane or THF, in the presence of an inorganic base such as K₂PO₄ or cesium carbonate, with a suitable palladium catalytic system such as Pd₂(dppf)Cl₂ or another palladium source/phosphine based ligand at high temperature (around 100° C.) for a few hours.

Direct amidation of esters (ammonolysis) may be carried on between Intermediate III and Intermediate IXa to obtain Intermediate IV, using for example potassium tert-butoxide or as sodium methoxide as a promoter in a suitable organic solvent as THF or Dioxane at room temperature for few hours.

In a different approach, intermediate IV may be prepared with a one-step synthesis starting from intermediate IX, under suitable amide coupling reaction conditions. For example, intermediate IX and IXa may be reacted in the presence of an activating agent such as COMU or HATU, with an organic base such as DIPEA or TEA, in a suitable organic solvent such as DCM or DMF, and at temperature generally around RT for a time ranging from a few hours to overnight.

Palladium-catalyzed reductive carbonylation of aryl halides may be performed starting from Intermediate IV to prepare Intermediate V, when R₄=H. For example formic acid or formylsaccharine may be used as CO sources, with silane or formic acid itself as the hydrogen donor and suitable palladium catalytic system such as Palladium acetate/Ph₃P or Palladium acetate/bis(diphenylphosphino)butane or another palladium source/phosphine based ligand, TEA or Na₂CO₃ as a base, in a suitable solvent as Toluene or DMF, at a temperature ranging from 60 to 100° C. for a few hours.

Alternatively, Intermediate V may be prepared from Intermediate XX under suitable amide coupling reaction conditions, as described for preparation of Intermediate IV. Intermediate V can be also prepared from Intermediate XX, by converting it into the acyl chloride XXI using for example thionyl chloride or oxalyl chloride, in a suitable solvent such as DCM, and performing subsequently an amide coupling using a suitable base, such as DIPEA or TEA, in a suitable solvent, such DCM or DMF, at room temperature.

In a different approach, when R=CHF₂, Intermediate XX can be prepared from intermediate XXV through for example ester hydrolysis, using LiOH in a suitable solvent, such as THF or Dioxane, at room temperature.

Intermediate XXV can be obtained via ozonolysis, applying for example an ozone stream in a suitable solvent such as DCM and performing a suitable reductive work-up, such as using Ph₃P or Me₂S, at a suitable temperature, such as zero degrees.

Deoxofluorination of Intermediate XXII to afford Intermediate XXIII, can be carried out in a solvent such as DCM or DMF, in presence of a fluorinating agent such as DAST or Deoxo-Fluor reagent, at a suitable temperature such as room temperature. Pd-catalyzed cross-coupling, may be carried out by reacting a halide-aryl intermediate XXIII, where halide is X₃, with an alkylboronic acid or potassium alkyltrifluoroborate following the classical Suzuki protocol, in a suitable organic solvent such as Dioxane or THF, in the presence of an inorganic base such as K₂PO₄ or cesium carbonate, with a suitable palladium catalytic system such as Pd₂(dppf)Cl₂ or another palladium source/phosphine based ligand at high temperature (around 100° C.) for a few hours, to afford Intermediate XXIV. Reductive amination of Intermediate V with the amine R₂—NH₂ (when R₄=H), to afford Intermediate VII, may be carried out in a solvent such as 1,2-Dichloroethane or DCM, in presence of a reductant such as NaBH₃CN or Na(OAc)₃BH, at room temperature.

Differently, Intermediate VII can be prepared via a two-step synthesis in which the imminic intermediate VI is formed first reacting Intermediate V with amine R₂—NH₂ in a suitable solvent such as 1,2-Dichloroethane, DCM or toluene at room temperature or at reflux if required. The presence of dehydrating agent can help the formation of the imine that is than converted into VII by addition of reducing agent as above described.

Intermediate VI can also be useful to prepare intermediate VII when R₄=alkyl or cycloalkyl, by mean of a 1,2-addition of a suitable organometallic reagent such as Grignard reagent or organolithium reagent, at a temperature ranging from 78° C. to room temperature.

In a further method, Intermediate VII, when R₄=alkyl or cycloalkyl, may be prepared converting Intermediate V into aminic Intermediate VIII, performing a reductive amination with an ammonia source such as ammonium acetate or ammonia solution and a reductant such as NaBH₃CN or NaBH₄ in a suitable solvent such as MeOH or EtOH at a temperature ranging from room temperature to 50° C. Then Intermediate VIII may undergo a Buchwald-Hartwig cross coupling reaction with a halide or triflate R₂—X (when R₂=heteroaryl), in a suitable organic solvent such as Dioxane or Toluene, in the presence of an inorganic base such as K₂PO₄ or Cesium Carbonate, with a suitable palladium catalytic system such as Pd(dba)2/RuPhos or another palladium source/phosphine based ligand at high temperature (around 100° C.) for a period ranging from few hours to overnight.

Alternatively, Ipso-substitution of the leaving group of the R₂—X (when R₂=heteroaryl) by the amine group of the intermediate VIII, to give intermediate VII, may be carried out in a high boiling organic solvent such as DMSO or DMA, at a temperature equal to or higher that 100° C. and in the presence of an inorganic base such as tBuOK or K₂CO₃.

Intermediate VII, when R₅=H, does not need further reaction to be converted into Compound I.

Intermediate VII may be converted into Compound of formula (I), when R₅ is different from H and Z is absent or CH₂, via reductive amination with an alkylic aldehyde R₅—CHO performed in a similar way to that described for the preparation of Intermediate VII from Intermediate V.

Alternatively, compounds of formula (I) may be prepared according to SCHEME 2 as described hereinafter providing at least one non-limiting synthetic route for the preparation of all examples.

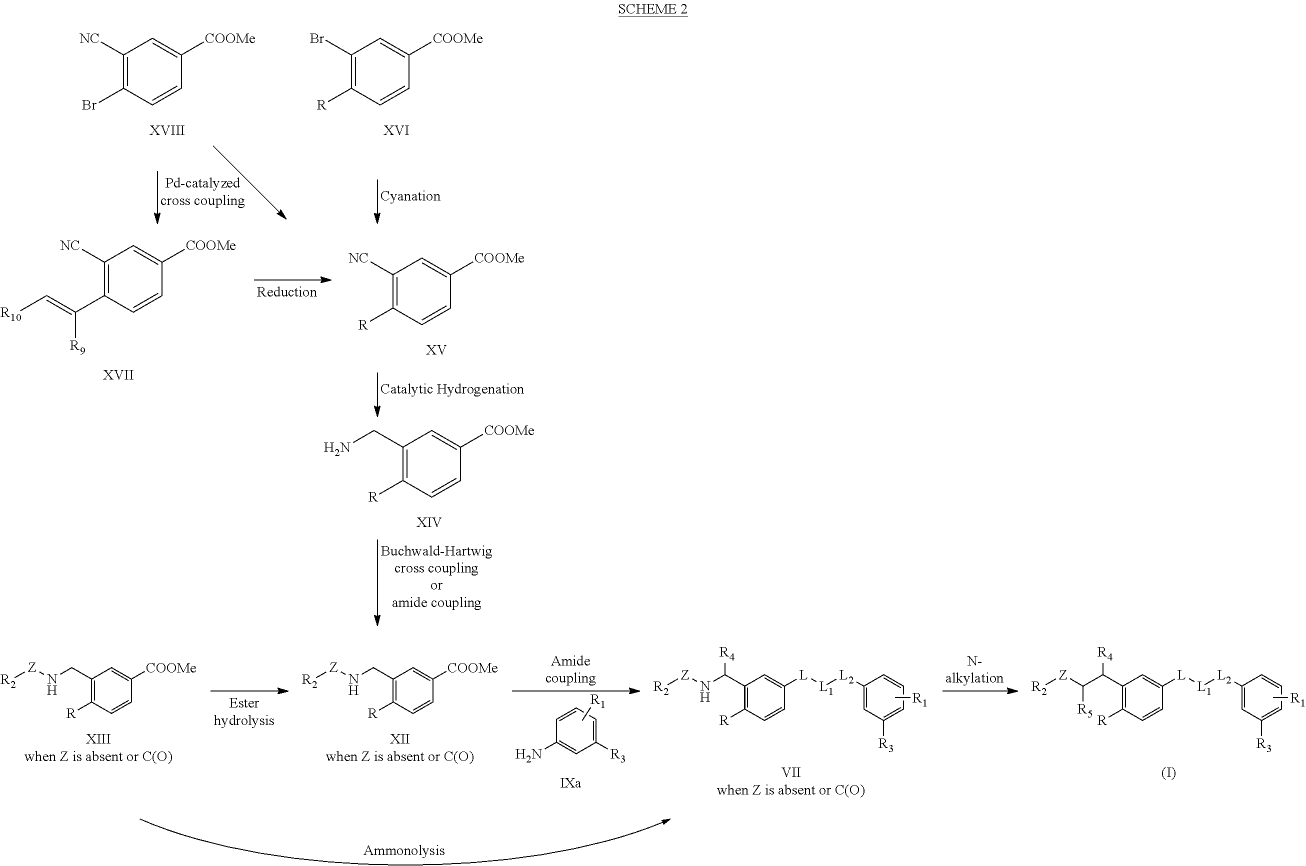

According to SCHEME 2, Intermediate XVIII can be converted into intermediate XV by Pd-catalyzed alkylation of aryl bromide by means of a Negishi, Stille or Suzuki cross-coupling, reacting XVIII with an alkylzinc halide or alkylstannane in the presence of a suitable organic solvent such as THF or Toluene, with a suitable palladium catalytic system such as Pd(OAc)₂/CPhos or another palladium source/phosphine based ligand at high temperature (around 100° C.) for a period ranging from few hours to overnight.

Alternatively, Intermediate XV may be prepared through Intermediate XVII, obtained following a Suzuki protocol starting from Intermediate XVIII, using for example an alkenylboronic acid or vinyltrifluoroborate with a suitable palladium catalytic system such as PdCl₂(dppf), in presence of an inorganic base such as TEA or Cesium Carbonate, in a suitable solvent such as Dioxane or iPrOH at a high temperature (around 100° C.) for a period ranging from few hours to overnight. Then Intermediate XVII may be converted into Intermediate XV by reduction under hydrogen atmosphere in presence of a suitable catalyst such as Pd/C in a suitable solvent such as, but not limited to, EtOH at room temperature for few hours.

In a different approach Intermediate XV may be obtained from Intermediate XVI carrying out a Pd-catalyzed cyanation of the aryl halide, using for example zinc cyanide in a suitable solvent such as DMF or DMA and a suitable Pd catalyst such as Pd(PPh₃)₄ or XantPhos-PdCl₂, at a high temperature (around 100° C.).

Catalytic hydrogenation of Intermediate XV to give Intermediate XIV may be carried out under hydrogen atmosphere using for example Raney nickel or Platinum dioxide and ammonia or KOH in a suitable solvent such as MeOH or iPrOH at room temperature.

Intermediate XIV may be converted into Intermediate XIII performing a Buchwald-Hartwig cross-coupling reaction when Z is absent, using a halide or a triflate R₂—X (when R₂=heteroaryl), in a suitable organic solvent such as Dioxane or Toluene, in the presence of an inorganic base such as K₂PO₄ or Cesium Carbonate, with a suitable palladium catalytic system such as Pd(dba)₂/RuPhos or another palladium source/phosphine based ligand at high temperature (around 100° C.) for a period ranging from few hours to overnight. When Z=CO an amide coupling may be carried out using an activating agent such as COMU or HATU, with an organic base such as DIPEA or TEA, in a suitable organic solvent such as DCM or DMF, and at temperature generally around RT for a time ranging from a few hours to overnight.

Ester hydrolysis of Intermediate XIII may lead to Intermediate XII using an inorganic base such as LiOH or Ba(OH)₂ in a mixture of an organic solvent such as THF and/or methanol with water, generally at RT and for a time ranging from 1 h to overnight. Intermediate XII may be converted into Intermediate VII by amide coupling reaction with an amine IXa using an activating agent such as BTFFH or T3P, with an organic base such as DIPEA or TEA, in a suitable organic solvent such as DCM or DMF, and at temperature generally around RT for a time ranging from a few hours to overnight.

Direct amidation of esters (ammonolysis) may be carried on between Intermediate XIII and Intermediate IXa to obtain Intermediate VII, using for example potassium tert-butoxide or as sodium methoxide as a promoter in a suitable organic solvent as THF or Dioxane at room temperature for few hours.

Intermediate VII, when R₅=H, does not need further reaction to be converted into Compound of formula (I).

Intermediate VII may be converted into Compound of formula (I), when R₅ is different from H and Z is CO, performing an alkylation on the amidic nitrogen, using for example an alkyl halide or alkyltriflate R₅—X with a suitable base such as KOH or NaH in a suitable solvent such as DMSO or DMF

In a different approach, compounds of formula (I) may be prepared according to SCHEME 3 as described hereinafter providing at least one non-limiting synthetic route for the preparation of all examples.

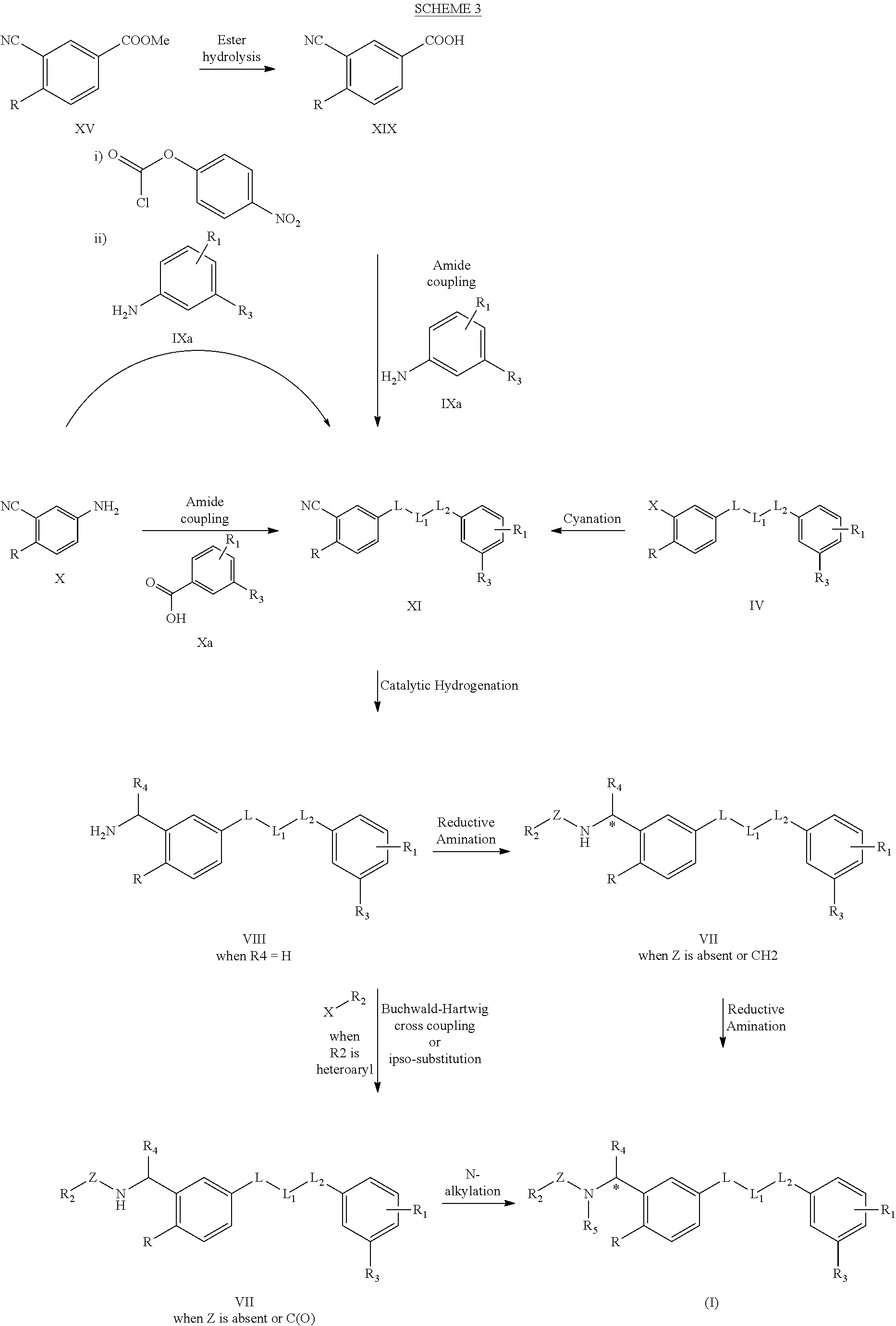

According to SCHEME 3, Intermediate VIII may be converted into Intermediate VII through reductive amination using an heteroarylaldehyde R₂—CHO, in a similar way to that described for the preparation of Intermediate VII from Intermediate V.

Intermediate VII (when Z is absent) may be obtained performing a Buchwald-Hartwig amination starting from Intermediate VIII in a similar way to that described above for the preparation of Intermediate XIII.

Alternatively, Intermediate VII may be prepared reacting Intermediate VIII and a fluoroaryl R₂—X performing an ipso-substitution using for example LiOH as a base in a suitable high boiling solvent such as DMF at a temperature ranging from room temperature to 100° C.

Catalytic hydrogenation of the cyano group on Intermediate XI, carried out in a similar way described above for the preparation of Intermediate XIV, may lead to Intermediate VIII. Intermediate XI when L=NH and L₂ is absent can be obtained through an amide coupling using Intermediate X and carboxylic acid Xa, in a similar way described above for the preparation of Intermediate XIII

Intermediate XI when L₂=NH may be prepared in a two-step process carrying out the formation of p-nitrocarbamate using p-nitrochloroformate with a suitable base such as Pyridine or TEA in a suitable solvent such as DCM at room temperature, followed by urea formation using with amine IXa, a suitable solvent such as DCM or DMF and a base such as DIPEA or TEA at room temperature.

In a different approach, Intermediate XI when L₂=CO may be obtained directly form intermediate XIX via amide coupling with Intermediate IXa in a similar way described above for the preparation of Intermediate XIII.

In a further alternative approach, Intermediate XI may be prepared via Pd-catalyzed cyanation from Intermediate IV, in a similar way described above for the preparation of Intermediate XV.

Intermediate VII, when R₅=H, does not need further reaction to be converted into Compound of formula (I).

Intermediate VII may be converted into Compound of formula (I), when R₅ is different from H and Z is CO, performing an alkylation on the amidic nitrogen, using for example an alkyl halide or alkyltriflate R₅—X with a suitable base such as KOH or NaH in a suitable solvent such as DMSO or DMF.

Alternatively, Intermediate VII may be converted into Compound of formula (I), when R₅ is different from H and Z is absent or CH₂, via reductive amination with an alkylic aldehyde R₅—CHO performed in a similar way to that described for the preparation of Intermediate VII from Intermediate V.

As above mentioned, the compound of formula (I) of the invention can conveniently be prepared by using common intermediates, represented by the compounds of formula VII and VIII.

In a further aspect, the invention refers to a compound of formula VIII

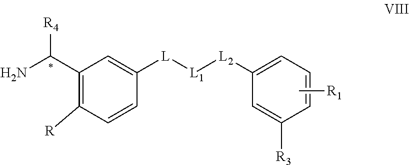

wherein R, R₁, R₃ and R₄ are as above indicated.

In a further aspect, the invention refers to a compound of formula VII

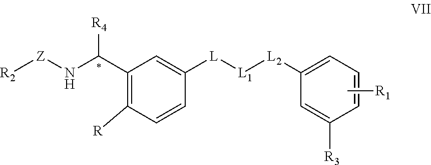

wherein Z is absent, CH₂ or —C(O), R, R₁, R₂, R₃ and R₄ are as above indicated.

In a further aspect, the invention refers to the use of the compound VII as intermediate for the preparation of a compound of formula (I), wherein Z is absent, CH₂ or —C(O), and R, R₁, R₂, R₃ and R₄ are as above indicated.

In a further aspect, the invention refers to the use of the compound VIII as intermediate for the preparation of a compound of formula (I).

The compounds of formula (I) of the present invention have surprisingly been found to effectively inhibit both receptor DDR1 and DDR2. Advantageously, the inhibition of receptors DDR1 and DDR2 may result in efficacious treatment of the diseases or condition wherein the DDR receptors are involved.

In particular in this respect, it has now been found that the compounds of formula (I) of the present invention have an antagonist drug potency expressed as inhibition constant (Ki) on DDR1 and DDR2 showed Ki values lower than 1000 nM and for most of the compounds of the invention Ki is even lower that 300 nM as shown in the present experimental part. Preferably, the compounds of the present invention have a Ki on DDR1 and DDR2 lesser or equal than 30 nM.

It has moreover been found that some compounds of formula (I) of the present invention have an inhibitory drug potency on DDR1 and DDR2 expressed as IC₅₀ lower than 15 nM and even more preferably lower than 10 nM.

In one aspect, the present invention refers to a compound of formula (I) for use as a medicament.

In a preferred embodiment, the invention refers to a compound of formula (I) for use in the treatment of disorders associated with DDR receptors mechanism.

In a further embodiment, the present invention refers to a compound of formula (I) for use in the treatment of a disease, disorder or condition associated with DDR receptors.

In one embodiment, the present invention refers to a compound of formula (I) useful for the prevention and/or treatment of fibrosis and/or diseases, disorders, or conditions that involve fibrosis.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Preferably, the compounds of formula (I) of the present invention are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis. More preferably, the compounds of formula (I) of the present invention are useful for the treatment of IPF.

In one aspect, the invention also refers to a method for the prevention and/or treatment of disorders associated with DDR receptors mechanisms, said method comprises administering to a patient in need of such treatment a therapeutically effective amount of a compound of formula (I).

In a further aspect, the invention refers to the use of a compound of formula (I) according to the invention for the treatment of disorders associated with DDR receptors mechanism.

In one aspect, the invention refers to the use of a compound of formula (I) in the preparation of a medicament for the treatment of disorders associated with DDR receptors mechanism.

In a further aspect, the invention refers to a method for the prevention and/or treatment of disorder or condition associated with dysregulation of DDR receptors 1 and 2 administering a patient in need of such treatment a therapeutically effective amount of a compound of formula (I).

In a further aspect, the present invention refers to the use of a compound of formula (I) for the treatment of a disease, disorder or condition associated with dysregulation of DDR receptors 1 and 2.

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) in admixture with at least one or more pharmaceutically acceptable carrier or excipient.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage forms such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of Formula (I) according to the invention, in form of a single- or multi-dose dry powder inhaler or a metered dose inhaler.

All preferred groups or embodiments described above for compounds of formula (I) may be combined among each other and apply as well mutatis mutandis.

The various aspects of the invention described in this application are illustrated by the following examples which are not meant to limit the invention in any way.

### PREPARATIONS OF INTERMEDIATES AND EXAMPLES

Chemical Names of the compounds were generated with Structure To Name Place IUPAC Name by PerkinElmer ChemDraw Professional 18.1.

The compounds of the invention can be prepared from readily available starting materials using the following general methods and procedures or by using other information readily available to those of ordinary skill in the art. Although a particular embodiment of the present invention may be shown or described herein, those skilled in the art will recognize that all embodiments or aspects of the present invention can be prepared using the methods described herein or by using other methods, reagents and starting materials known to those skilled in the art. It will also be appreciated that where typical or preferred process conditions (i.e., reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by one skilled in the art by routine optimization procedures.

Abbreviations

Et₃N=triethyl amine; TEA=triethylamine; HATU=(Dimethylamino)-N,N-dimethyl(3H-[1,2,3]triazolo[4,5-b]pyridin-3-yloxy)methaniminium hexafluorophosphate; DAST=Diethylamino sulfur trifluoride; DMAP=4-dimethylaminopyridine; DMF=dimethylformamide; Me₂S, or (CH₃)₂S=Methyl sulfide; MnO₂=Manganese(IV) oxide; EtOAc=Ethyl acetate; RT=room temperature; THF=tetrahydrofuran; DCM=dichloromethane; MeOH=methyl alcohol; LCMS=Liquid Chromatography/Mass Spectrometry; HPLC=high pressure liquid chromatography; TLC=Thin Layer Chromatography; d-DMSO=deuterated dimethyl sulfoxide. CDCl₃=deuterated chloroform; NMR=nuclear magnetic resonance; DIPEA=N,N-Diisopropylethylamine; UPLC=Ultra Performance Liquid Chromatography; tBu XPhos=2-Di-tert-butylphosphino-2′,4′,6′-triisopropylbiphenyl; Pd₂(dba)₃=Tris(dibenzylideneacetone)dipalladium(0); iPrOH=iso-propanol; PdCl₂(dppf)=[1,1′-Bis(diphenylphosphino)ferrocene]dichloropalladium(II); atm=atmospheres; RuPhos=2-Dicyclohexylphosphino-2′,6′-diisopropoxybiphenyl; Pd(dba)₂=Bis(dibenzylideneacetone)palladium(0); BINAP=(±)-2,2′-Bis(diphenylphosphino)-1,1′-binaphthalene; STAB=sodium triacetoxyborohydride; CPhos=2-Dicyclohexylphosphino-2′,6′-bis(N,N-dimethylamino)biphenyl; Pd(OAc)₂=palladium(II) acetate; AcOH=Acetic acid; Py=pyridine; T3P=Propanephosphonic acid anhydride; prepHPLC=preparative high pressure liquid chromatography; NaBH₄=sodium borohydride; Na₂SO₄=sodium sulfate; BTFFH=Fluoro-dipyrrolidinocarbenium hexafluorophosphate; pTLC=preparative thin layer chromatography; FCC=flash column chromatography; amu=atomic mass unit; t_{R}=retention time; FA=Formic acid

General Experimental Details

NMR Characterization

¹H NMR spectra were recorded on Bruker Avance III HD 400 MHz or Bruker Fourier 300 MHz. Chemical shifts are reported as δ values in ppm relative to tetramethyl silane (TMS) as an internal standard. Coupling constants (J values) are given in hertz (Hz) and multiplicities are reported using the following abbreviation (s=singlet, d=doublet, t=triplet, q=quartet, m=multiplet, br=broad, nd=not determined).

LC/UV/MS Analytical Methods

LC/MS retention times are estimated to be affected by an experimental error of ±0.5 min.

Method 1 (LCMS-019-20-80-95-6-1-25-UV-BCM)

Apparatus: Dionex UHPLC Ultimate 3000 with DAD detector/Thermo Scientific MSQ Plus

Column: Kinetex® 2.6 μm XB-C18 (4.6×50 mm), 110A, column no. 00B-4496-E0, internal column no. 019

Reagents:

Formic acid≥98%, Sigma-Aldrich
Acetonitrile for HPLC UV/gradient grade, Baker

### μQ-water for LCMS

HPLC Conditions:

Wavelength range: (190-340) nm±4 nm
Flow: 1.0 ml/min
Column temperature: 25° C.
Autosampler temperature: 20° C.
Injection volume: 2.0 μl
Analysis time: 6 min
Elution: gradient

| | Mobile phase A | Mobile phase B | |
|---|---|---|---|
| Time [min] | [%] | [%] | Flow [ml/min] |
| 0.0 | 80 | 20 | 1.0 |
| 3.35 | 20 | 80 | 1.0 |
| 3.75 | 20 | 80 | 1.0 |
| 3.9 | 5 | 95 | 1.0 |
| 4.75 | 5 | 95 | 1.0 |
| 5.0 | 80 | 20 | 1.0 |
| 6.0 | 80 | 20 | 1.0 |

Mobile phase A: 0.1% v/v water solution of formic acid
Mobile phase B: 0.1% v/v acetonitrile solution of formic acid
Solution for syringe washing: 20% MeOH

MS conditions:

Mass range: 100-1000 m/z
Ionization: alternate
Scan speed: 12 000 amu/sec

Method 2 (LCMS-019-10-60-95-6-1-25-UV)

Apparatus: Dionex UHPLC Ultimate 3000 with DAD detector/Thermo Scientific MSQ Plus

Column: Kinetex® 2.6 μm XB-C₁₈ (4.6×50 mm), 110A, column no. 00B-4496-E0, internal column no. 019

Reagents:

Formic acid≥98%, Sigma-Aldrich
Acetonitrile for HPLC UV/gradient grade, Baker

### μQ-water for LCMS

HPLC Conditions:

Wavelength range: (190-340) nm±4 nm
Flow: 1.0 ml/min
Column temperature: 25° C.
Autosampler temperature: 20° C.
Injection volume: 2.0 μl
Analysis time: 6 min
Elution: gradient

| | Mobile phase A | Mobile phase B | |
|---|---|---|---|
| Time [min] | [%] | [%] | Flow [ml/min] |
| 0.0 | 90 | 10 | 1.0 |
| 3.35 | 40 | 60 | 1.0 |
| 3.75 | 40 | 60 | 1.0 |
| 3.9 | 5 | 95 | 1.0 |
| 4.75 | 5 | 95 | 1.0 |
| 5.0 | 90 | 10 | 1.0 |
| 6.0 | 90 | 10 | 1.0 |

Mobile phase A: 0.1% v/v water solution of formic acid
Mobile phase B: 0.1% v/v acetonitrile solution of formic acid
Solution for syringe washing: 20% MeOH

Ms Conditions:

Mass range: 100-1000 m/z
Ionization: alternate
Scan speed: 12 000 amu/sec

Method 3 (LCMS-019-5-80-80-7-1-25-UV-Rot)

Apparatus: Dionex UHPLC Ultimate 3000 with DAD detector/Thermo Scientific MSQ Plus

Column: Kinetex® 2.6 μm XB-C18 (4.6×50 mm), 110A, column no. OOB-4496-E0, internal column no. 019

Reagents:

Formic acid≥98%, Sigma-Aldrich
Acetonitrile for HPLC UV/gradient grade, Baker

### μQ-water for LCMS

HPLC Conditions:

Wavelength range: (190-340) nm±4 nm
Flow: 1.0 ml/min
Column temperature: 25° C.
Autosampler temperature: 20° C.
Analysis time: 7 min
Elution: gradient

| | Mobile phase A | Mobile phase B | |
|---|---|---|---|
| Time [min] | [%] | [%] | Flow [ml/min] |
| 0.0 | 95 | 5 | 1.0 |
| 1.0 | 95 | 5 | 1.0 |
| 4.75 | 20 | 80 | 1.0 |
| 5.25 | 20 | 80 | 1.0 |
| 6.0 | 95 | 5 | 1.0 |
| 7.0 | 95 | 5 | 1.0 |

Mobile phase A: 0.1% v/v water solution of formic acid
Mobile phase B: 0.1% v/v acetonitrile solution of formic acid
Solution for syringe washing: 20% MeOH

Ms Conditions:

Mass range: 100-1000 m/z
Ionization: alternate
Scan speed: 12 000 amu/sec

Method 4 (LCMS-019-10-70-95-6-1-25-UV)

Apparatus: Dionex UHPLC Ultimate 3000 with DAD detector/Thermo Scientific MSQ Plus

Column: Kinetex® 2.6 μm XB-C18 (4.6×50 mm), 110A, column no. 00B-4496-E0, internal column no. 019

Reagents:

Formic acid≥98%, Sigma-Aldrich
Acetonitrile for HPLC UV/gradient grade, Baker

### μQ-water for LCMS

HPLC Conditions:

Wavelength range: (190-340) nm±4 nm
Flow: 1.0 ml/min
Column temperature: 25° C.
Autosampler temperature: 20° C.
Injection volume: 2.0 μl
Analysis time: 6 min
Elution: gradient

| | Mobile phase A | Mobile phase B | |
|---|---|---|---|
| Time [min] | [%] | [%] | Flow [ml/min] |
| 0.0 | 90 | 10 | 1.0 |
| 3.35 | 30 | 70 | 1.0 |
| 3.75 | 30 | 70 | 1.0 |
| 3.9 | 5 | 95 | 1.0 |
| 4.75 | 5 | 95 | 1.0 |
| 5.0 | 90 | 10 | 1.0 |
| 6.0 | 90 | 10 | 1.0 |

Mobile phase A: 0.1% v/v water solution of formic acid
Mobile phase B: 0.1% v/v acetonitrile solution of formic acid
Solution for syringe washing: 20% MeOH

MS conditions:

Mass range: 100-1000 m/z
Ionization: alternate
Scan speed: 12 000 amu/sec

Method 5 (LCMS-005-1-30-50-10-05-55 UV)

Apparatus: Dionex UHPLC Ultimate 3000 with DAD detector/Thermo Scientific MSQ Plus

Column: ACQUITY UPLC BEH C8 1.7 μm (2.1×150 mm), 130 A, column no. 186003377, internal column no. 005

Reagents:

Formic acid≥98%, Sigma-Aldrich
Acetonitrile for HPLC UV/gradient grade, Baker

### μQ-water for LCMS

HPLC Conditions:

Wavelength range: (190-340) nm±4 nm
Flow: 0.5 ml/min
Column temperature: 55° C.
Autosampler temperature: 20° C.
Analysis time: 10 min
Elution: gradient

| | Mobile phase A | Mobile phase B | |
|---|---|---|---|
| Time [min] | [%] | [%] | Flow [ml/min] |
| 0.0 | 99 | 1 | 0.5 |
| 0.5 | 99 | 1 | 0.5 |
| 3.0 | 70 | 30 | 0.5 |
| 6.5 | 50 | 50 | 0.5 |
| 7.5 | 20 | 80 | 0.5 |
| 8.0 | 20 | 80 | 0.5 |
| 8.1 | 99 | 1 | 0.5 |
| 10.0 | 99 | 1 | 0.5 |

Mobile phase A: 0.1% v/v water solution of formic acid
Mobile phase B: 0.1% v/v acetonitrile solution of formic acid
Solution for syringe washing: 20% MeOH

Ms Conditions:

Mass range: 100-1000 m/
Ionization: alternate
Scan speed: 12 000 amu/sec

Method 6: (LCMS-019-30-80-95-6-1-25-UV)

Apparatus: Dionex UHPLC Ultimate 3000 with DAD detector/Thermo Scientific MSQ Plus

Column: Kinetex® 2.6 μm XB-C18 (4.6×50 mm), 110A, column no. 00B-4496-E0, internal column no. 019

Reagents:

Formic acid≥98%, Sigma-Aldrich
Acetonitrile for HPLC UV/gradient grade, Baker

### μQ-water for LCMS

HPLC Conditions:

Wavelength range: (190-340) nm±4 nm
Flow: 1.0 ml/min
Column temperature: 25° C.
Autosampler temperature: 20° C.
Analysis time: 6 min
Elution: gradient

| | Mobile phase A | Mobile phase B | |
|---|---|---|---|
| Time [min] | [%] | [%] | Flow [ml/min] |
| 0.0 | 70 | 30 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 70 | 30 | 1.0 |
| 6.00 | 70 | 30 | 1.0 |

Mobile phase A: 0.1% v/v water solution of formic acid
Mobile phase B: 0.1% v/v acetonitrile solution of formic acid
Solution for syringe washing: 20% MeOH

MS conditions:

Mass range: 100-1000 m/z
Ionization: alternate
Scan speed: 12 000 amu/sec

Where the preparation of starting materials is not described, these are commercially available, known in the literature, or readily obtainable by those skilled in the art using standard procedures. All solvents were purchased from commercial sources and were used without additional purification.

Thin layer chromatography was performed on Merck silica gel 60 F254 TLC plates.

Preparative thin-layer chromatography (pTLC) was performed with Uniplate 1000 micron or 500 micron silica gel plates. Flash chromatography was performed on Interchim PuriFlash 450 and 520Plus systems using pre-packed silica gel cartridges.

When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions. All final compounds were obtained as a free base, unless stated otherwise.

General Method a for Amide Coupling

Carboxylic acid or carboxylic acid salt (1.0 eq), amine (1.0 eq.) and DIPEA (6.0 eq) were dissolved in anhydrous DCM under argon. Next, T3P (50% in EtOAc, 1.5 eq.) was added and the reaction was stirred at RT overnight. The reaction mixture was partitioned between DCM and water. The water phase was extracted with DCM (3×) and the combined organic phases were concentrated to afford the crude product which was purified by the indicated method.

General Method B for Amide Coupling

Carboxylic acid or carboxylic acid salt (1.0 eq) was dissolved in anhydrous DMF under argon, then BTFFH (3.0 eq) and DIPEA (4.5 eq) were added. Next, amine (1.5 eq) was added and the reaction was stirred at 80° C. overnight. Then, the reaction mixture was concentrated to dryness in vacuo and the residue was partitioned between EtOAc and water. The water phase was extracted with EtOAc (3×), the combined organic phases were washed with brine and concentrated to afford crude product which was purified by the indicated method.

General Method C for Amide Coupling:

Carboxylic acid salt (1.0 eq) and amine (1.0 eq.) were dissolved the mixture of DMF:DCM (1:3), followed by the addition of DIPEA (8.0 eq.) and HATU (2.0 eq.). The reaction was stirred overnight at RT, then the reaction mixture was partitioned between DCM and saturated NaHCO₃. The water phase was extracted with DCM (3×), the combined organic layers were dried with Na₂SO₄ and concentrated in vacuo to afford the crude product which was purified by the indicated method.

### Scheme for the Preparation of Intermediate lithio 3-[(4-methylpiperazin-1-yl)methyl]-5-(trifluoromethyl)benzoate

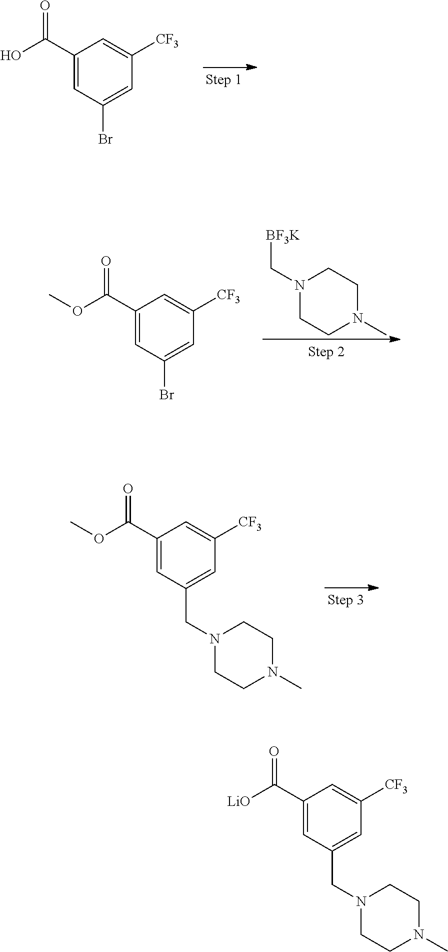

Step 1: Preparation of methyl 3-bromo-5-(trifluoromethyl)benzoate

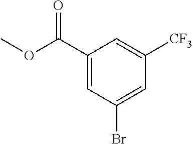

To the solution of 3-bromo-5-(trifluoromethyl)benzoic acid (75.0 g, 279 mmol) in MeOH (282 mL), SOCl₂ (81.0 mL, 1115 mmol) was added dropwise at 0° C. Next, the reaction mixture was stirred under reflux overnight, whereupon volatiles were removed in vacuo. To the residue water (200 mL) was added and aqueous layer was extracted with EtOAc (2×250 mL). Combined organic layers were washed with saturated solution of NaHCO₃ (2×200 mL), dried over Na₂SO₄ concentrated in vacuo to afford the product (74.5 g, 94%) as beige solid.

¹H NMR (300 MHz, DMSO-d6) δ 8.30 (dt, J=1.8, 0.8 Hz, 2H), 8.13 (td, J=1.6, 0.8 Hz, 1H), 3.90 (s, 3H).

Step 2: Preparation of methyl 3-[(4-methylpiperazin-1-yl)methyl]-5-(trifluoromethyl)benzoate

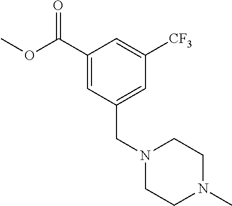

3-Bromo-5-(trifluoromethyl)benzoate (47.5 g, 168 mmol), Cs₂CO₃ (164 g, 503 mmol), potassium 1-methyl-4-trifluoroboratomethylpiperazine (40.6 g, 184.6 mmol) were suspended in a mixture of THF (100 mL) and water (11 mL). The suspension was degassed, then Pd(OAc)₂ (3.76 g, 16.8 mmol) and XPhos (16.0 g, 33.5 mmol) were added and the reaction was carried out at 80° C. for 24 h. The reaction mixture was diluted with water (100 mL) and extracted with EtOAc (2×150 mL). The combined organic phases were concentrated and dried in vacuo to afford the crude, which was purified by column chromatography (DCM:MeOH, 9:1) to give titular compound as a brown oil (25.3 g, 48%).

¹H NMR (300 MHz, DMSO-d6) δ 8.16 (d, J=1.7 Hz, 1H), 8.07 (t, J=1.8 Hz, 1H), 7.97-7.86 (m, 1H), 3.90 (s, 3H), 3.62 (s, 2H), 2.38 (s, 8H), 2.15 (s, 3H).

Step 3: Preparation of lithio 3-[(4-methylpiperazin-1-yl)methyl]-5-(trifluoromethyl)benzoate

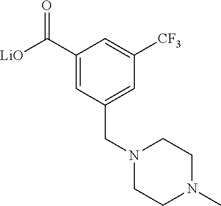

Methyl 3-[(4-methylpiperazin-1-yl)methyl]-5-(trifluoromethyl)benzoate (25.3 g, 80.0 mmol) was dissolved in MeOH (700 mL). 1M LiOH solution was added (3.8 g, 160 mL) to the reaction mixture and stirred at RT overnight. The solvent was removed in vacuo and the crude material triturated with diethyl ether (2×) and filtered. The solid was collected to give the titular product as a solid (26.0 g, 100%).

¹H NMR (300 MHz, DMSO-d6) δ 8.02 (s, 2H), 7.49 (s, 1H), 3.51 (s, 2H), 2.32 (s, 8H), 2.14 (s, 3H).

### Scheme for the Preparation of intermediate 5-(1-methyl-1H-pyrazol-3-yl)pyridin-3-amine

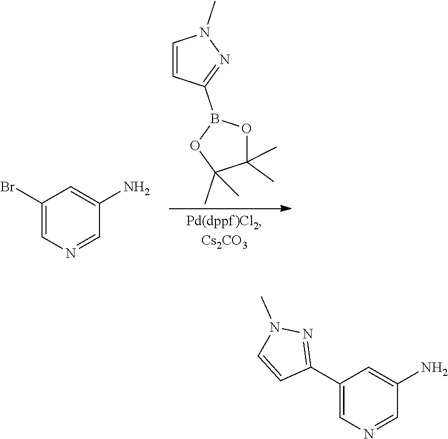

Step 1: Preparation of 5-(1-methyl-1H-pyrazol-3-yl)pyridin-3-amine

5-bromopyridin-3-amine (0.5 g, 2.89 mmol), 1-methyl-3-(4,4,5,5-tetramethyl-1,3,2-dioxaborolan-2-yl)-1H-pyrazole (0.782 g, 3.76 mmol) and Cs₂CO₃ (2.82 g, 8.67 mmol) were suspended in Dioxane (11.42 ml) and water (1.142 ml). The mixture was purged with Ar for 15 min, then Pd(dppf)Cl₂ (0.211 g, 0.289 mmol) was added. The reaction mixture was stirred for 3 h at 90° C., then cooled down to RT and concentrated. The crude material was purified via FCC (eluting system: from 100% to 10% MeOH in DCM). The compound was triturated with Et₂O to give the desired product (468 mg, 93%).

¹H NMR (300 MHz, DMSO-d6) δ 8.20-8.10 (m, 1H), 7.84 (d, J=2.6 Hz, 1H), 7.73 (d, J=2.2 Hz, 1H), 7.31 (dd, J=2.6, 1.8 Hz, 1H), 6.62 (d, J=2.3 Hz, 1H), 5.33 (s, 2H), 3.87 (s, 3H).

### Example 1

4-methyl-N-(3-(4-methyl-1H-imidazol-1-yl)-5-(trifluoromethyl)phenyl)-3-((pyrazolo[1,5-a]pyrimidin-6-ylamino)methyl)benzamide

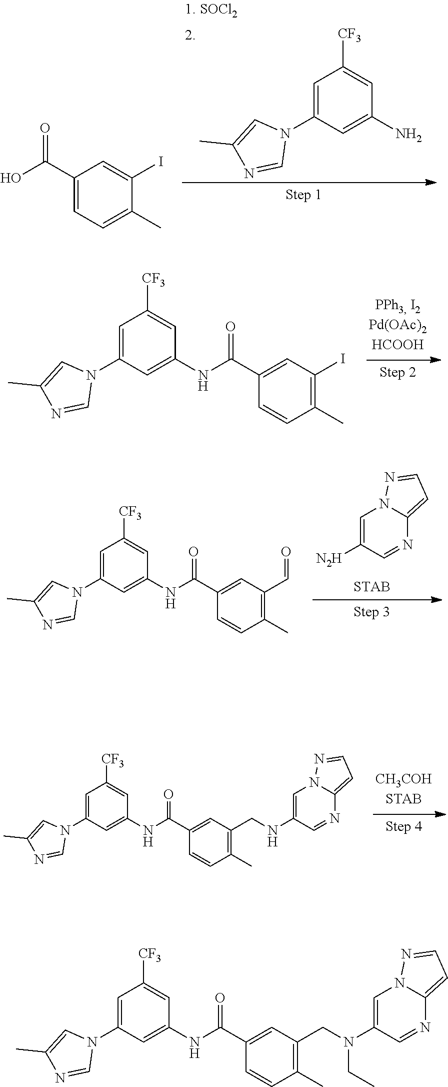

Step 1: Preparation of 3-iodo-4-methyl-N-[3-(4-methyl-1H-imidazol-1-yl)-5-(trifluoromethyl)phenyl]benzamide

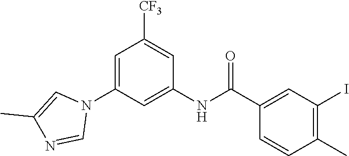

A solution of 3-iodo-4-methylbenzoic acid (7.00 g, 26.7 mmol) in SOCl₂ (47 mL) was refluxed for 2 h, then evaporated in vacuo to remove residual SOCl₂. The residue was dissolved in anhydrous THF (25 mL) and added dropwise to a solution of DIPEA (4.14 g, 32.0 mmol), 3-(4-methyl-1H-imidazol-1-yl)-5-(trifluoromethyl)aniline (6.44 g, 26.7 mmol) and DMAP (130 mg, 1.06 mmol) in anhydrous THF (48 mL). The reaction mixture was stirred at RT for 17 h and evaporated in vacuo. The residue was dissolved in EtOAc (200 mL). Water was added (180 mL) and the pH was adjusted to 8 with 1 M NaOH. The layers were then separated and aqueous layer was extracted with DCM/MeOH 100:5 (100 mL×5). The combined organic extracts were evaporated in vacuo to give the final product as off-white solid (13.05 g, 100%).

¹H NMR (300 MHz, DMSO-d6) δ 10.67 (s, 1H), 8.46 (d, J=1.9 Hz, 1H), 8.27 (t, J=1.9 Hz, 1H), 8.21 (d, J=1.4 Hz, 1H), 8.13 (d, J=1.8 Hz, 1H), 7.94 (dd, J=7.9, 1.9 Hz, 1H), 7.74 (d, J=1.8 Hz, 1H), 7.53 (d, J=8.0 Hz, 1H), 7.49 (d, J=1.6 Hz, 1H), 2.46 (s, 3H), 2.18 (d, J=1.0 Hz, 3H).

Step 2: Preparation of 3-formyl-4-methyl-N-[3-(4-methyl-1H-imidazol-1-yl)-5-(trifluoromethyl)phenyl]benzamide

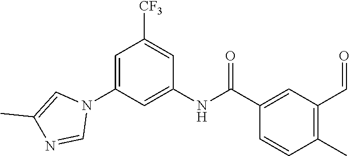 [SMILES: Cc1cn(-c2cc(NC(=O)c3ccc(C)c(C=O)c3)cc(C(F)(F)F)c2)cn1]

PPh₃ (1.62 g, 6.2 mmol), 12 (1.57 g, 6.2 mmol) and toluene (20 mL) were added to a 100 mL SealTube equipped with a stir bar and were stirred for 10 min at RT. Then 3-iodo-4-methyl-N-[3-(4-methyl-1H-imidazol-1-yl)-5-(trifluoromethyl)phenyl]benzamide (2.50 g, 5.15 mmol), Pd(OAc)₂ (34.7 mg, 3 mol %) and Et₃N (3.13 g, 30.9 mmol) were added into the solution. Next HCOOH (0.95 g, 20.6 mmol) was added, the tube was immediately sealed and the mixture was stirred at 80° C. for 4 h. The reaction mixture was cooled to RT, diluted with EtOAc (150 mL) and washed with 0.01M NaOH. The aqueous layer was extracted with EtOAc (150 mL×2), the combined extracts were dried over Na₂SO₄, filtered and concentrated under reduced pressure. The residue obtained was purified by column chromatography on silica gel (DCM/MeOH from 100:1 to 100:4) to provide the product as yellow solid (1.055 g, 53%).

¹H NMR (300 MHz, DMSO-d6) δ 10.83 (s, 1H), 10.35 (s, 1H), 8.48 (d, J=2.1 Hz, 1H), 8.30 (d, J=2.0 Hz, 1H), 8.22 (d, J=1.4 Hz, 1H), 8.18 (dd, J=8.0, 1.8 Hz, 1H), 8.16-8.14 (m, 1H), 7.75 (s, 1H), 7.59 (s, 1H), 7.50 (t, J=1.3 Hz, 1H), 2.73 (s, 3H), 2.18 (d, J=1.0 Hz, 3H).

Step 3: Preparation of 4-methyl-N-(3-(4-methyl-1H-imidazol-1-yl)-5-(trifluoromethyl)phenyl)-3-((pyrazolo[1,5-a]pyrimidin-6-ylamino)methyl)benzamide

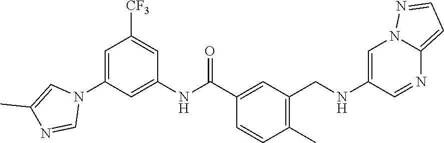 [SMILES: Cc1cn(-c2cc(NC(=O)c3ccc(C)c(CNc4cnc5ccnn5c4)c3)cc(C(F)(F)F)c2)cn1]

### Example 1

3-formyl-4-methyl-N-[3-(4-methyl-1H-imidazol-1-yl)-5-(trifluoromethyl)phenyl]benzamide (190 mg, 0.49 mmol) and pyrazolo[1,5-a]pyrimidin-6-amine (65.8 mg, 0.49 mmol) were dissolved in glacial AcOH (2.0 mL). The brown solution was stirred at RT for 3 h. Next, STAB (208 mg, 0.98 mmol) was added and the reaction mixture was stirred at RT overnight. AcOH was evaporated under reduced pressure, the residue dissolved in 1 M NaOH (25 mL) and the product extracted with EtOAc (50 mL) and DCM:MeOH 100:5 (50 mL×2). The combined extracts were evaporated under reduced pressure and the solid residue purified by column chromatography (DCM:MeOH, from 100:4 to 100:8) to provide the product as yellow solid (215 mg, 86%).

| | t_{R} | | |
|---|---|---|---|
| Example | (min) | Method | NMR data |
| Example 1 | 1.963 | 1 | ¹H NMR (400 MHz, DMSO-d6) δ 10.61 |
| | | | (s, 1H), 8.40 (d, J = 2.6 Hz, 1H), 8.28 (t, J = |
| | | | 2.0 Hz, 1H), 8.19 (d, J = 1.5 Hz, 1H), 8.13- |
| | | | 8.10 (m, 2H), 7.99 (d, J = 1.9 Hz, 1H), 7.89 |
| | | | (dd, J = 7.9, 2.1 Hz, 1H), 7.87 (d, J = 2.4 |
| | | | Hz, 1H), 7.71 (d, J = 2.2 Hz, 1H), 7.47 (t, |
| | | | J = 1.3 Hz, 1H), 7.44 (d, J = 8.0 Hz, 1H), |
| | | | 6.54 (dd, J = 2.4, 0.8 Hz, 1H), 6.40 (t, J = |
| | | | 5.6 Hz, 1H), 4.32 (d, J = 5.2 Hz, 2H), 2.46 |
| | | | (s, 3H), 2.17 (d, J = 1.0 Hz, 3H). |

### Example 2

3-((ethyl(pyrazolo[1,5-a]pyrimidin-6-yl)amino)methyl)-4-methyl-N-(3-(4-methyl-1H-imidazol-1-yl)-5-(trifluoromethyl)phenyl)benzamide

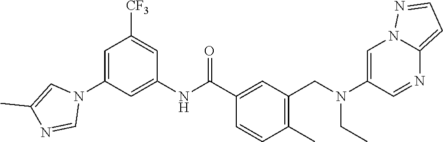 [SMILES: CCN(Cc1cc(C(=O)Nc2cc(-n3cnc(C)c3)cc(C(F)(F)F)c2)ccc1C)c1cnc2ccnn2c1]

### Example 2

4-methyl-N-(3-(4-methyl-1H-imidazol-1-yl)-5-(trifluoromethyl)phenyl)-3-((pyrazolo[1,5-a]pyrimidin-6-ylamino)methyl)benzamide (Example 1) (30.0 mg, 0.059 mmol) was dissolved in glacial AcOH (0.4 mL) and acetaldehyde (0.10 mL, 1.78 mmol) was added at RT. Next, STAB (25.2 mg, 0.12 mmol) was added and the reaction mixture was stirred at RT overnight. The reaction mixture was treated with 0.1 M NaOH (10 mL) and the product extracted with DCM (30 mL×3). The combined extracts were dried over Na₂SO₄, filtered and evaporated under reduced pressure. The solid residue was purified by prepHPLC to provide the product as light yellow solid (5 mg, 16%).

| | t_{R} | | |
|---|---|---|---|
| Example | (min) | Method | Analytical dataNMR |
| Example 2 | 2.220 | 1 | ¹H NMR (300 MHz, Methanol-d4) δ 8.48 |
| | | | (d, J = 2.7 Hz, 1H), 8.19 (t, J = 2.0 Hz, 1H), |
| | | | 8.17-8.12 (m, 1H), 8.10 (d, J = 1.5 Hz, 1H), |
| | | | 8.02 (s, 1H), 7.90 (d, J = 2.5 Hz, 1H), 7.89 |
| | | | (d, J = 1.8 Hz, 1H), 7.83 (dd, J = 7.8, 2.0 |
| | | | Hz, 1H), 7.59 (s, 1H), 7.41 (d, J = 7.9 Hz, |
| | | | 1H), 7.34 (s, 1H), 6.54 (dd, J = 2.5, 0.9 Hz, |
| | | | 1H), 4.56 (s, 2H), 3.56 (q, J = 7.0 Hz, 2H), |
| | | | 2.49 (s, 3H), 2.25 (d, J = 1.0 Hz, 3H), 1.24 |
| | | | (t, J = 7.0 Hz, 3H). |

The following compounds were prepared via reductive amination as described for Example 1, step 1-3, applying the corresponding commercially available amines in step 3.

| | | | Amount of | | | Analytical |
|---|---|---|---|---|---|---|
| | Structure and | Amount | product | | t_{R} | data |
| Ex. | Chemical Name | of SM | (yield) | Method | (min) | NMR |
| Ex. 3 | 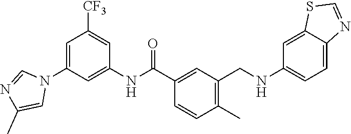 [SMILES: Cc1cn(-c2cc(NC(=O)c3ccc(C)c(CNc4ccc5ncsc5c4)c3)cc(C(F)(F)F)c2)cn1] | 0.20 g | 0.036 g (Y = 13%) | 1 | 1.963 | ¹H NMR (300 MHz, Methanol-d4) δ 8.80 (s, 1H), 8.21 (t, J = 2.1 Hz, 1H), 8.10 (d, J = 1.5 Hz, 1H), 8.05 (s, 1H), 8.02 (d, J = |
| | 3-((benzo[d]thiazol-6- | | | | | 2.0 Hz, 1H), |
| | ylamino)methyl)-4-methyl- | | | | | 7.82 (dd, J = |
| | N-(3-(4-methyl-1H- | | | | | 7.9, 2.0 Hz, |
| | imidazol-1-yl)-5- | | | | | 1H), 7.77 (d, J = |
| | (trifluoromethyl)phenyl) | | | | | 8.9 Hz, 1H), |
| | benzamide | | | | | 7.59 (s, 1H), |
| | | | | | | 7.39 (d, J = 8.0 |
| | | | | | | Hz, 1H), 7.35 (t, |
| | | | | | | J = 1.3 Hz, 1H), |
| | | | | | | 7.11 (d, J = 2.3 |
| | | | | | | Hz, 1H), 6.96 |
| | | | | | | (dd, J = 8.9, 2.3 |
| | | | | | | Hz, 1H), 4.44 |
| | | | | | | (s, 2H), 2.50 (s, |
| | | | | | | 3H), 2.26 (d, J = |
| | | | | | | 1.1 Hz, 3H). |
| Ex. 4 | 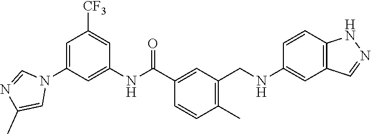 [SMILES: Cc1cn(-c2cc(NC(=O)c3ccc(C)c(CNc4ccc5[nH]ncc5c4)c3)cc(C(F)(F)F)c2)cn1] | 0.20 g | 0.020 g (Y = 7.7%) | 4 | 1.683 | ¹H NMR (300 MHz, Methanol-d4) δ 8.22 (s, 1H), 8.11 (d, J = 1.5 Hz, 1H), 8.05 (d, J = 3.6 Hz, 2H), 7.81 (dd, J = 7.9, 2.0 Hz, |
| | 3-(((1H-indazol-5- | | | | | 1H), 7.77 (s, |
| | yl)amino)methyl)-4- | | | | | 1H), 7.60 (s, |
| | methyl-N-(3-(4-methyl- | | | | | 1H), 7.39 (d, J = |
| | 1H-imidazol-1-yl)-5- | | | | | 8.0 Hz, 1H), |
| | (trifluoromethyl)phenyl) | | | | | 7.34 (d, J = 8.5 |
| | benzamide | | | | | Hz, 2H), 7.01 |
| | | | | | | (dd, J = 8.9, 2.2 |
| | | | | | | Hz, 1H), 6.77 |
| | | | | | | (s, 1H), 4.39 (s, |
| | | | | | | 2H), 2.51 (s, |
| | | | | | | 3H), 2.26 (d, J = |
| | | | | | | 1.0 Hz, 3H). |
| Ex. 5 | 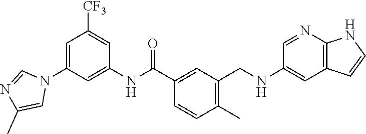 [SMILES: Cc1cn(-c2cc(NC(=O)c3ccc(C)c(CNc4cnc5[nH]ccc5c4)c3)cc(C(F)(F)F)c2)cn1] | 0.20 g | 0.026 g (Y = 10%) | 4 | 2.173 | ¹H NMR (300 MHz, Methanol-d4) δ 8.21 (d, J = 2.1 Hz, 1H), 8.11 (d, J = 1.5 Hz, 1H), 8.08-8.02 (m, 2H), 7.81 (dd, J = 7.5, 2.1 |
| | 3-(((1H-pyrrolo[2,3- | | | | | Hz, 2H), 7.60 |
| | b]pyridin-5- | | | | | (s, 1H), 7.39 (d, |
| | yl)amino)methyl)-4- | | | | | J = 8.0 Hz, 1H), |
| | methyl-N-(3-(4-methyl- | | | | | 7.36 (s, 1H), |
| | 1H-imidazol-1-yl)-5- | | | | | 7.25 (d, J = 2.5 |
| | (trifluoromethyl)phenyl) | | | | | Hz, 1H), 7.23 |
| | benzamide | | | | | (d, J = 3.4 Hz, |
| | | | | | | 1H), 6.27 (d, J = |
| | | | | | | 3.4 Hz, 1H), |
| | | | | | | 4.40 (s, 2H), |
| | | | | | | 2.51 (s, 3H), |
| | | | | | | 2.26 (d, J = 1.1 |
| | | | | | | Hz, 3H). |

### Example 6

4-methyl-N-(3-(4-methyl-1H-imidazol-1-yl)-5-(trifluoromethyl)phenyl)-3-(((4,5,6,7-tetrahydropyrazolo[1,5-a]pyrimidin-6-yl)amino)methyl)benzamide

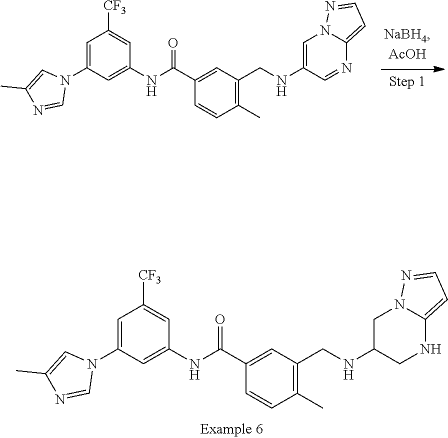 [SMILES: Cc1cn(-c2cc(NC(=O)c3ccc(C)c(CNc4cnc5ccnn5c4)c3)cc(C(F)(F)F)c2)cn1 ; Cc1cn(-c2cc(NC(=O)c3ccc(C)c(CNC4CNc5ccnn5C4)c3)cc(C(F)(F)F)c2)cn1]

4-methyl-N-(3-(4-methyl-1H-imidazol-1-yl)-5-(trifluoromethyl)phenyl)-3-((pyrazolo[1,5-a]pyrimidin-6-ylamino)methyl)benzamide (Example 1) (30.0 mg, 0.059 mmol) was dissolved in glacial AcOH (0.4 mL) and NaBH₄ (9 mg, 0.24 mmol) was added at RT. The reaction mixture was stirred at RT overnight. AcOH was evaporated under reduced pressure, the residue dissolved in 0.4 M NaOH (7 mL) and the product extracted with DCM (20 mL×3). The combined extracts were evaporated under reduced pressure and the solid residue purified by pTLC (DCM:MeOH, 100:4) to provide the product as colorless solid (16 mg, 53%).

| | t_{R} | | |
|---|---|---|---|
| Example | (min) | Method | Analytical data NMR |
| Ex. 6 | 1.627 | 2 | ¹H NMR (300 MHz, DMSO-d6) δ 10.61 |
| | | | (s, 1H), 8.31 (t, J = 2.0 Hz, 1H), 8.20 (d, |
| | | | J = 1.4 Hz, 1H), 8.15 (d, J = 2.1 Hz, 1H), |
| | | | 7.98 (d, J = 2.0 Hz, 1H), 7.82 (dd, J = 7.8, |
| | | | 2.0 Hz, 1H), 7.72 (d, J = 1.9 Hz, 1H), 7.49 |
| | | | (t, J = 1.3 Hz, 1H), 7.35 (d, J = 8.0 Hz, 1H), |
| | | | 7.05 (d, J = 1.9 Hz, 1H), 6.01 (t, J = 2.8 |
| | | | Hz, 1H), 5.17 (d, J = 1.9 Hz, 1H), 4.19 (dd, |
| | | | J = 12.0, 4.8 Hz, 1H), 3.88 (s, 2H), 3.78 |
| | | | (dd, J = 12.0, 7.4 Hz, 1H), 3.32 (dd, J = |
| | | | 11.3, 3.0 Hz, 1H), 3.21-3.09 (m, 1H), 3.05- |
| | | | 2.95 (m, 1H), 2.39 (s, 3H), 2.18 (d, J = 1.0 |
| | | | Hz, 3H). |

### Example 7

3-(((2-cyanopyridin-4-yl)amino)methyl)-4-methyl-N-(3-(4-methyl-1H-imidazol-1-yl)-5-(trifluoromethyl)phenyl)benzamide

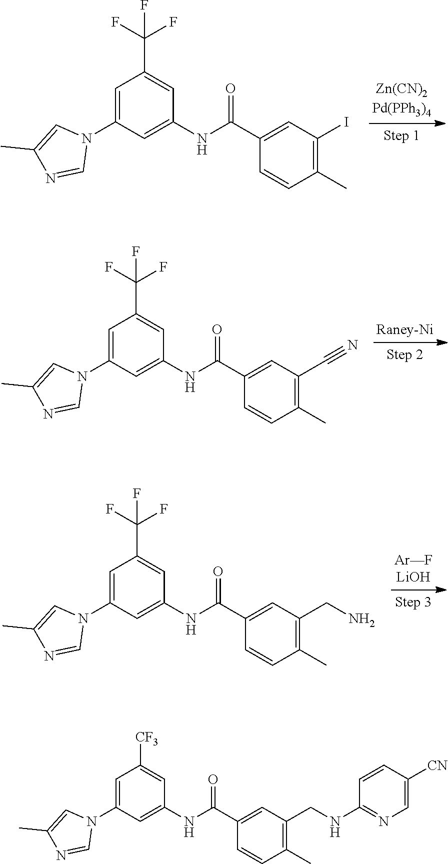 [SMILES: Cc1cn(-c2cc(NC(=O)c3ccc(C)c(I)c3)cc(C(F)(F)F)c2)cn1 ; Cc1cn(-c2cc(NC(=O)c3ccc(C)c(C#N)c3)cc(C(F)(F)F)c2)cn1 ; Cc1cn(-c2cc(NC(=O)c3ccc(C)c(CN)c3)cc(C(F)(F)F)c2)cn1 ; Cc1cn(-c2cc(NC(=O)c3ccc(C)c(CNc4ccc(C#N)cn4)c3)cc(C(F)(F)F)c2)cn1]

Step 1: Preparation of 3-cyano-4-methyl-N-(3-(4-methyl-1H-imidazol-1-yl)-5-(trifluoromethyl)phenyl)benzamide

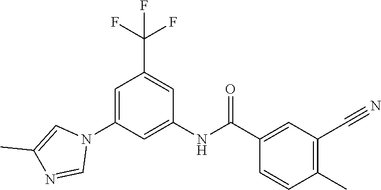 [SMILES: Cc1cn(-c2cc(NC(=O)c3ccc(C)c(C#N)c3)cc(C(F)(F)F)c2)cn1]

3-iodo-4-methyl-N-(3-(4-methyl-1H-imidazol-1-yl) (trifluoromethyl)phenyl)benzamide (7 g, 14.43 mmol), prepared as described in Example 1, step 1, zinc cyanide (2.028 g, 17.31 mmol) and Pd(PPh₃)₄ (0.834 g, 0.721 mmol) were dissolved in DMF (48.1 ml). The reaction mixture was stirred at 80° C. for 4 hr. The mixture was diluted with EtOAc and washed with water. The organic solvents were then removed under reduced pressure. The crude material was purified via dry flash chromatography (hexane:EtOAc, 9:1) to afford the product as a beige powder (6.7 g, quant.)

¹H NMR (300 MHz, DMSO-d6) δ 10.78 (s, 1H), 8.42 (d, J=1.9 Hz, 1H), 8.27 (t, J=1.9 Hz, 1H), 8.22 (d, J=1.4 Hz, 1H), 8.19 (dd, J=8.1, 2.0 Hz, 1H), 8.13 (d, J=1.9 Hz, 1H), 7.77 (d, J=2.2 Hz, 1H), 7.69 (d, J=8.2 Hz, 1H), 7.51 (t, J=1.3 Hz, 1H), 2.59 (s, 3H), 2.19 (d, J=1.0 Hz, 3H).

Step 2: Preparation fo 3-(aminomethyl)-4-methyl-N-(3-(4-methyl-1H-imidazol-1-yl)-5-(trifluoromethyl)phenyl)benzamide

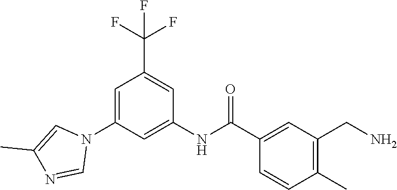 [SMILES: Cc1cn(-c2cc(NC(=O)c3ccc(C)c(CN)c3)cc(C(F)(F)F)c2)cn1]

3-Cyano-4-methyl-N-(3-(4-methyl-1H-imidazol-1-yl)-5-(trifluoromethyl)phenyl)benzamide (2.2 g, 5.72 mmol) was dissolved in MeOH (45.8 ml) and ammonia (11.45 ml), then Raney nickel (2 ml, 5.72 mmol) was added. The reaction mixture was stirred under hydrogen atmosphere (balloon) over 3 d. The reaction mixture was filtered through a pad of Celite, concentrated and dried in vacuo to afford the crude product (1.62 g, 67.6%) which was used in the next step without further purification.

¹H NMR (300 MHz, DMSO-d6) δ 10.59 (s, 1H), 8.31 (s, 1H), 8.19 (d, J=14.4 Hz, 2H), 8.03 (s, 1H), 7.78 (d, J=7.4 Hz, 1H), 7.72 (s, 1H), 7.50 (s, 1H), 7.33 (d, J=7.9 Hz, 1H), 3.80 (s, 2H), 2.36 (s, 3H), 2.18 (s, 3H).

Step 3: Preparation of 3-(((2-cyanopyridin-4-yl)amino)methyl)-4-methyl-N-(3-(4-methyl-1H-imidazol-1-yl)-5-(trifluoromethyl)phenyl)benzamide (Example 7)

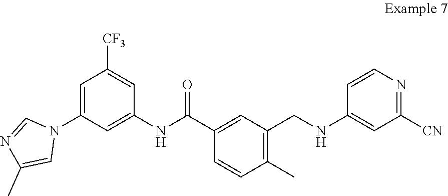 [SMILES: Cc1cn(-c2cc(NC(=O)c3ccc(C)c(CNc4ccnc(C#N)c4)c3)cc(C(F)(F)F)c2)cn1]

3-(aminomethyl)-4-methyl-N-(3-(4-methyl-1H-imidazol-1-yl)-5-(trifluoromethyl) phenyl) benzamide (0.1 g, 0.257 mmol) and 4-fluoropicolinonitrile (0.038 g, 0.309 mmol) were dissolved in DMF (0.52 ml). Then LiOH (0.013 g, 0.548 mmol) was added and the reaction mixture was stirred at rt overnight. Reaction mixture was diluted with water and extracted with AcOEt (×3). The organic layers were combined, dried over Na₂SO₄, filtered and concentrated. The crude material was purified via FCC (from 100% DCM to 10% MeOH in DCM) to give the desired product as a white solid (67 mg, 53%).

| | t_{R} | | |
|---|---|---|---|
| Example | (min) | Method | Analytical data NMR |
| Ex. 7 | 1.627 | 4 | ¹H NMR (300 MHz, DMSO-d6) δ 10.60 |
| | | | (s, 1H), 8.28 (t, J = 2.0 Hz, 1H), 8.19 (d, |
| | | | J = 1.4 Hz, 1H), 8.15 (d, J = 5.9 Hz, 1H), |
| | | | 8.12 (s, 1H), 7.89 (dd, J = 8.0, 1.9 Hz, |
| | | | 1H), 7.87 (s, 1H), 7.72 (s, 1H), 7.60 (t, |
| | | | J = 5.5 Hz, 1H), 7.48 (t, J = 1.3 Hz, 1H), |
| | | | 7.43 (d, J = 7.8 Hz, 1H), 7.14 (d, J = 2.4 |
| | | | Hz, 1H), 6.81 (d, J = 5.9 Hz, 1H), 4.42 |
| | | | (d, J = 5.5 Hz, 2H), 2.41 (s, 3H), 2.18 |
| | | | (d, J = 1.0 Hz, 3H). |

The Example 8 was prepared according to the above protocol using the appropriate fluoro-arylamine.

| | | | Amount of | | | Analytical |
|---|---|---|---|---|---|---|
| | | Amount | product | | t_{R} | data |
| Ex. | Structure | of SM | (yield) | Method | (min) | NMR |
| Ex. 8 | 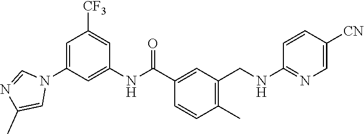 [SMILES: Cc1cn(-c2cc(NC(=O)c3ccc(C)c(CNc4ccc(C#N)cn4)c3)cc(C(F)(F)F)c2)cn1] | 0.10 g | 0.085 g (Y = 67%) | 7 | 2.220 | ¹H NMR (300 MHz, DMSO-d6) δ 10.59 (s, 1H), 8.46-8.37 (m, 1H), 8.28 (t, J = 2.0 Hz, 1H), 8.19 (d, J = 1.4 Hz, 1H), 8.12 (s, 1H), |
| | 3-(((5-cyanopyridin-2- | | | | | 8.06 (t, J = 5.6 Hz, |
| | yl)amino)methyl)-4- | | | | | 1H), 7.86 (d, J = |
| | methyl-N-(3-(4-methyl- | | | | | 7.5 Hz, 2H), 7.76- |
| | 1H-imidazol-1-yl)-5- | | | | | 7.67 (m, 2H), |
| | (trifluoromethyl)phenyl) | | | | | 7.51-7.44 (m, |
| | benzamide | | | | | 1H), 7.40 (d, J = |
| | | | | | | 7.8 Hz, 1H), 6.66 |
| | | | | | | (d, J = 8.9 Hz, |
| | | | | | | 1H), 4.60 (d, J = |
| | | | | | | 5.5 Hz, 2H), 2.40 |
| | | | | | | (s, 3H), 2.18 (d, |
| | | | | | | J = 1.0 Hz, 3H). |

### Example 9

N-(4-methyl-3-((pyrimidin-5-ylamino)methyl)phenyl)-3-((4-methylpiperazin-1-yl)methyl)-5-(trifluoromethyl)benzamide

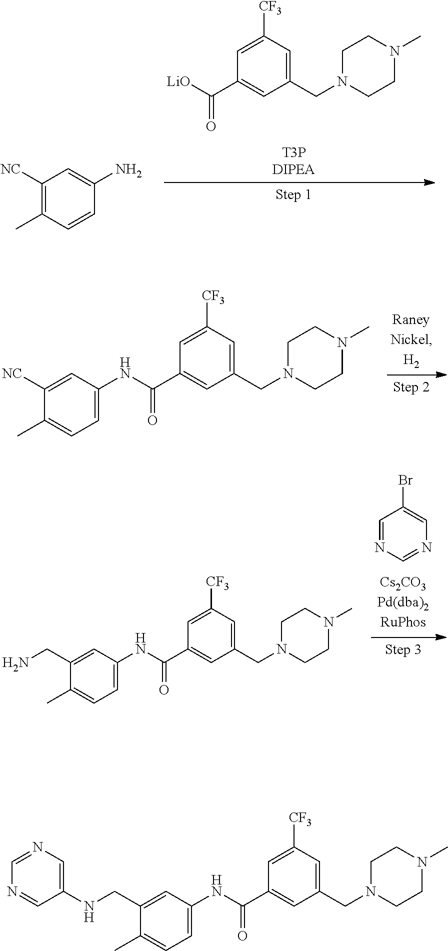

Step 1: Preparation of N-(3-cyano-4-methylphenyl)-3-((4-methylpiperazin yl)methyl)-5-(trifluoromethyl)benzamide

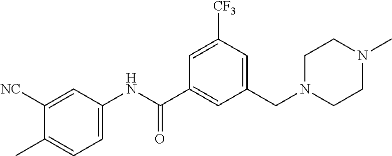

5-amino-2-methylbenzonitrile (0.500 g, 3.78 mmol) and lithium 3-((4-methylpiperazin-1-yl)methyl)-5-(trifluoromethyl)benzoate (1.166 g, 3.78 mmol) were dissolved in DCM (38 mL), then DIPEA (3.96 mL, 22.70 mmol) and 50% T3P in EtOAc (3.34 mL, 5.67 mmol) were added. The reaction mixture was stirred at 40° C. for 24 h, and afterwards, diluted with DCM and washed with water (3×50 mL). The aqueous phase was then extracted with DCM (3×50 mL). The combined organic layers were washed with brine (100 mL) and concentrated in vacuo. The crude material was purified via column chromatography (DCM:MeOH, from 100:0 to 90:10) to give the titular compound (0.342 g, 22% yield) as a reddish-white solid.

¹H NMR (300 MHz, DMSO-d6) δ 10.67 (s, 1H), 8.26-8.13 (m, 3H), 7.97-7.87 (m, 2H), 7.49 (d, J=8.5 Hz, 1H), 3.65 (s, 2H), 2.47 (s, 3H), 2.38 (d, J=21.9 Hz, 8H), 2.16 (s, 3H).

Step 2: Preparation of N-(3-(aminomethyl)-4-methylphenyl)-3-((4-methylpiperazin-1-yl)methyl)-5-(trifluoromethyl)benzamide

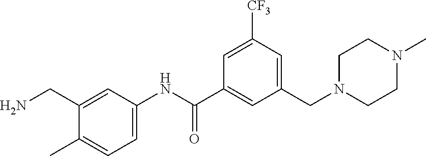 [SMILES: Cc1ccc(NC(=O)c2cc(CN3CCN(C)CC3)cc(C(F)(F)F)c2)cc1CN]

A solution of N-(3-cyano-4-methylphenyl)-3-((4-methylpiperazin-1-yl)methyl)-5-(trifluoromethyl)benzamide (0.345 g, 0.828 mmol) in MeOH (8.28 mL) was charged with Raney Nickel (1.6 ml, 0.828 mmol) and was stirred at RT in the presence of hydrogen atmosphere (balloon) for 16 h. The reaction mixture was filtered through a pad of Celite, concentrated and dried in vacuo to afford the crude product (334 mg, 96% yield) as a yellow solid, which was used in the following step without additional purification.

¹H NMR (300 MHz, DMSO-d6) δ 10.41 (d, J=10.1 Hz, 1H), 8.19 (d, J=9.4 Hz, 2H), 7.84 (s, 1H), 7.70 (d, J=2.2 Hz, 1H), 7.59 (dd, J=8.1, 2.3 Hz, 1H), 7.11 (d, J=8.3 Hz, 1H), 3.71 (s, 2H), 3.63 (s, 2H), 2.37 (m, 8H), 2.24 (s, 3H), 2.24 (m, 2H), 2.15 (s, 3H).

Step 3: Preparation of N-(4-methyl-3-((pyrimidin-5-ylamino)methyl)phenyl) ((4-methylpiperazin-1-yl)methyl)-5-(trifluoromethyl)benzamide (Example 9)

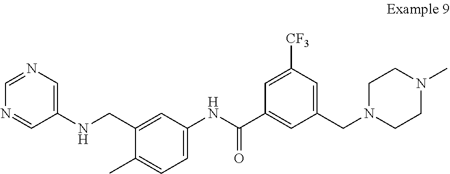 [SMILES: Cc1ccc(NC(=O)c2cc(CN3CCN(C)CC3)cc(C(F)(F)F)c2)cc1CNc1cncnc1]

N-(3-(aminomethyl)-4-methylphenyl)-3-((4-methylpiperazin-1-yl)methyl)-5-(trifluoromethyl)benzamide (110 mg, 0.262 mmol) 5-bromopyrimidine (49.9 mg, 0.314 mmol) and Cs₂(CO)₃ (256 mg, 0.785 mmol) were suspended in toluene (2 mL). The mixture was degassed with argon then RuPhos (24.42 mg, 0.052 mmol) and Pd(dba)₂ (15.04 mg, 0.026 mmol) were added and the reaction was stirred for 16 h at 110° C. The reaction was partitioned between water and DCM, the product was extracted with DCM (×3) and the combined organic layers were washed with brine and concentrated in vacuo, to give the crude material which was purified by FCC (DCM:MeOH, from 100:0 to 90:10) to afford the titular compound (18.00 mg, 14% yield) as a yellow solid.

| | t_{R} | | |
|---|---|---|---|
| Ex. | (min) | Method | Analytical dataNMR |
| Ex. 9 | 1.683 | 1 | ¹H NMR (300 MHz, Chloroform-d) δ 8.64 (s, |
| | | | 1H), 8.15 (s, 2H), 8.12 (s, 2H), 8.10 (br, 1H), |
| | | | 8.04 (s, 1H), 7.73 (s, 1H), 7.69 (s, 1H), 7.57 (d, |
| | | | J = 8.3 Hz, 1H), 7.23 (d, J = 8.2 Hz, 1H), 4.34 |
| | | | (d, J = 5.2 Hz, 2H), 4.20 (d, J = 5.4 Hz, 1H), |
| | | | 3.70 (s, 2H), 2.75 (s, 8H), 2.51 (s, 3H), 2.36 (s, |
| | | | 3H). |

### Example 10

N-methyl-4-((2-methyl-5-(3-((4-methylpiperazin-1-yl)methyl) (trifluoromethyl)benzamido)benzyl)amino)picolinamide

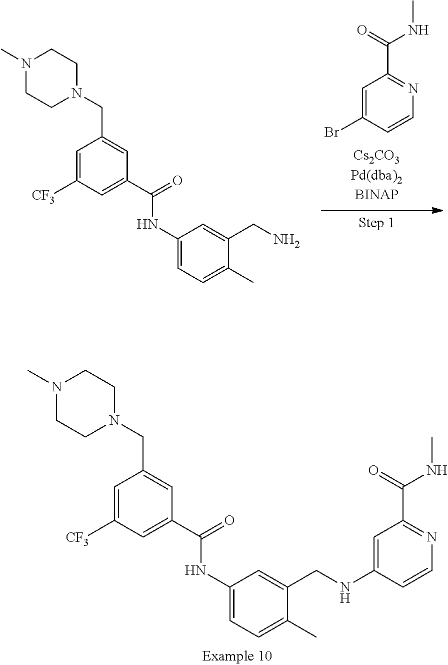

N-(3-(aminomethyl)-4-methylphenyl)-3-((4-methylpiperazin-1-yl)methyl)-5-(trifluoromethyl)benzamide (110 mg, 0.262 mmol), prepared as described in Example 9, step 1-2, 4-bromo-N-methylpicolinamide (67.5 mg, 0.314 mmol) and Cs₂(CO)₃ (256 mg, 0.785 mmol) were suspended in toluene (2 mL). The mixture was degassed with argon then BINAP (32.6 mg, 0.052 mmol and Pd(dba)₂ (15.04 mg, 0.026 mmol) were added and the reaction was stirred for 16 h at 110° C. The reaction was partitioned between water and DCM, the compound was extracted with DCM (×3) and the combined organic layers were washed with brine and concentrated in vacuo, to give the crude material which was purified by FCC (DCM:MeOH, from 100:0 to 90:10), followed by trituration with pentane to afford the titular compound (0.04 g, 28% yield) as a beige solid.

| | t_{R} | | |
|---|---|---|---|
| Ex. | (min) | Method | Analytical data NMR |
| Ex 10 | 1.88 | 2 | ¹H NMR (300 MHz, DMSO-d6) δ 10.39 (s, |
| | | | 1H), 8.55 (d, J = 5.0 Hz, 1H), 8.14 (s, 1H), |
| | | | 8.11 (s, 1H), 8.06 (d, J = 5.7 Hz, 1H), 7.82 |
| | | | (s, 1H), 7.71-7.54 (m, 2H), 7.34 (t, J = 5.5 |
| | | | Hz, 1H), 7.28-7.17 (m, 2H), 6.62 (s, 1H), |
| | | | 4.32 (d, J = 5.5 Hz, 2H), 3.61 (s, 2H), 2.76 |
| | | | (d, J = 4.8 Hz, 3H), 2.44-2.27 (m, 8H), 2.31 |
| | | | (s, 3H), 2.15 (s, 3H). |

### Example 11

N-(4-methyl-3-(((pyrimidin-5-ylmethyl)amino)methyl)phenyl)-3-((4-methylpiperazin-1-yl)methyl)-5-(trifluoromethyl)benzamide

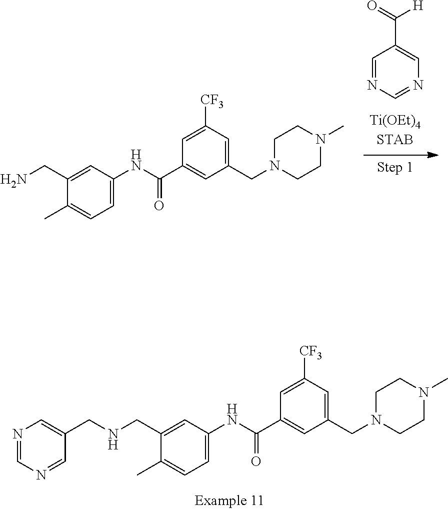 [SMILES: O=Cc1cncnc1 ; Cc1ccc(NC(=O)c2cc(CN3CCN(C)CC3)cc(C(F)(F)F)c2)cc1CN ; Cc1ccc(NC(=O)c2cc(CN3CCN(C)CC3)cc(C(F)(F)F)c2)cc1CNCc1cncnc1]

N-(3-(aminomethyl)-4-methylphenyl)-3-((4-methylpiperazin-1-yl)methyl)-5-(trifluoromethyl)benzamide (0.07 g, 0.166 mmol) prepared as described in Example 9, step 1-2, and pyrimidine-5-carbaldehyde (0.018 g, 0.166 mmol) were mixed and the tube was backfilled with argon (×3). THF (1.7 mL) was added to the mixture followed by Ti(OEt)₄ (0.070 mL, 0.333 mmol). The reaction mixture was cooled to 0° C. and STAB (0.141 g, 0.666 mmol) was added. The reaction mixture was warmed to RT and stirred for 16 h. The reaction mixture was added to a 1M NaOH solution. The desired product was then extracted with EtOAc (3×10 mL) and the combined organic layers were washed with brine (1×10 mL) and concentrated in vacuo, to give the crude material which was purified by column chromatography (DCM:MeOH, from 100:0 to 90:10) to give the titular compound (22.98 mg, 27% yield) as a yellow solid.

| | t_{R} | | |
|---|---|---|---|
| Ex. | (min) | Method | Analytical data NMR |
| Ex. 11 | 2.78 | 3 | ¹H NMR (300 MHz, Methanol-d4) δ 9.07 (s, |
| | | | 1H), 8.86 (s, 2H), 8.18 (s, 2H), 7.90 (s, 1H), |
| | | | 7.71 (d, J = 2.3 Hz, 1H), 7.49 (dd, J = 8.2, 2.3 |
| | | | Hz, 1H), 7.19 (d, J = 8.2 Hz, 1H), 3.93 (s, |
| | | | 2H), 3.84 (s, 2H), 3.73 (s, 2H), 2.61 (br s, |
| | | | 8H), 2.37 (s, 3H), 2.34 (s, 3H). |

The following compounds were prepared via reductive amination as described for Example 11, reacting the corresponding, commercially available aldehydes.

| | | | Amount of | | | |
|---|---|---|---|---|---|---|
| | Structure and | Amount | product | | t_{R} | |
| Ex. | Chemical Name | of SM | (yield) | Method | (min) | NMR data |
| Ex. 12 | 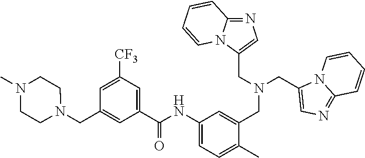 [SMILES: Cc1ccc(NC(=O)c2cc(CN3CCN(C)CC3)cc(C(F)(F)F)c2)cc1CN(Cc1cnc2ccccn12)Cc1cnc2ccccn12] | 0.1 g | 0.037 g (Y = 23%) | 4 | 1.58 | ¹H NMR (300 MHz, Methanol-d4) δ 8.17 (s, 2H), 7.90 (s, 1H), 7.80 (d, J = 2.3 Hz, 1H), 7.59 (s, 2H), 7.57-7.44 (m, 3H), 7.32-7.11 (m, 5H), 6.54 (td, J = 6.8, 1.2 Hz, 2H), 4.06 (s, 4H), 3.76 (s, 2H), 3.72 (s, 2H), 2.57 (br s, 8H), |
| | N-(3-((bis(imidazo[1,2- | | | | | 2.32 (s, 3H), 1.71 (s, |
| | a]pyridin-3- | | | | | 3H). |
| | ylmethyl)amino)methyl)- | | | | | |
| | 4-methylphenyl)-3- | | | | | |
| | ((4-methylpiperazin-1- | | | | | |
| | yl)methyl)-5- | | | | | |
| | (trifluoromethyl) | | | | | |
| | benzamide | | | | | |
| Ex. 13 | 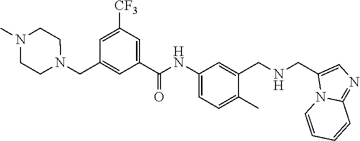 [SMILES: Cc1ccc(NC(=O)c2cc(CN3CCN(C)CC3)cc(C(F)(F)F)c2)cc1CNCc1cnc2ccccn12] | 0.1 g | 0.025 g (Y = 19%) | 3 | 2.60 | ¹H NMR (300 MHz, Methanol-d4) δ 8.47 (d, J = 6.9 Hz, 1H), 8.17 (s, 2H), 7.90 (s, 1H), 7.67 (s, 1H), 7.57 (s, 1H), 7.54 (d, J = 1.2 Hz, 1H), 7.48 (dd, J = 8.1, 2.3 Hz, 1H), 7.38-7.30 (m, 1H), |
| | N-(3-(((imidazo[1,2- | | | | | 7.17 (d, J = 8.2 Hz, |
| | a]pyridin-3- | | | | | 1H), 6.97 (td, J = 6.7, |
| | ylmethyl)amino)methyl)- | | | | | 1.1 Hz, 1H), 4.22 (s, |
| | 4-methylphenyl)-3- | | | | | 2H), 3.83 (s, 2H), 3.72 |
| | ((4-methylpiperazin-1- | | | | | (s, 2H), 2.56 (br s, |
| | yl)methyl)-5- | | | | | 8H), 2.32 (s, 3H), 2.28 |
| | (trifluoromethyl) | | | | | (s, 3H). |
| | benzamide | | | | | |
| Ex. 14 | 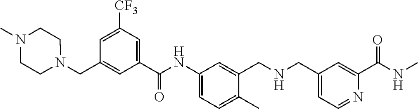 | 0.1 g | 0.027 g (Y = 20%) | 3 | 2.91 | ¹H NMR (300 MHz, Methanol-d4) δ 8.57 (d, J = 4.9 Hz, 1H), 8.21-8.12 (m, 3H), 7.90 (s, 1H), 7.69-7.51 (m, 3H), 7.19 (d, J = 8.3 Hz, 1H), 3.98 (s, |
| | N-methyl-4-(((2- | | | | | 2H), 3.81 (s, 2H), 3.72 |
| | methyl-5-(3-((4- | | | | | (s, 2H), 2.98 (s, 3H), |
| | methylpiperazin-1- | | | | | 2.57 (br s, 8H), 2.33 (s, |
| | yl)methyl)-5- | | | | | 3H), 2.32 (s, 3H). |
| | trifluoromethyl) | | | | | |
| | benzamido)benzyl)amino) | | | | | |
| | methyl)picolinamide | | | | | |
| Ex. 15 | 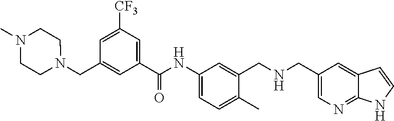 [SMILES: Cc1ccc(NC(=O)c2cc(CN3CCN(C)CC3)cc(C(F)(F)F)c2)cc1CNCc1cnc2[nH]ccc2c1] | 0.1 g | 0.043 g (Y = 33%) | 4 | 1.81 | ¹H NMR (300 MHz, Methanol-d4) δ 8.31 (d, J = 2.0 Hz, 1H), 8.19 (d, J = 2.8 Hz, 2H), 8.14 (d, J = 2.1 Hz, 1H), 7.91 (s, 1H), 7.78 (d, J = 2.2 Hz, 1H), 7.52 (dd, J = 8.2, |
| | N-(3-((((1H- | | | | | 2.3 Hz, 1H), 7.45 (d, |
| | pyrrolo[2,3-b]pyridin- | | | | | J = 3.5 Hz, 1H), 7.26 (d, |
| | 5-yl)methyl) | | | | | J = 8.3 Hz, 1H), 6.54 |
| | amino)methyl)- | | | | | (d, J = 3.5 Hz, 1H), |
| | 4-methylphenyl)-3- | | | | | 4.19 (s, 2H), 4.02 (s, |
| | ((4-methylpiperazin-1- | | | | | 2H), 3.73 (s, 2H), 2.62 |
| | yl)methyl)-5- | | | | | (s, 8H), 2.39 (s, 3H), |
| | (trifluoromethyl) | | | | | 2.33 (s, 3H). |
| | benzamide | | | | | |

### Example 16

4-isopropyl-N-(3-((4-methylpiperazin-1-yl)methyl)-5(trifluoromethyl) phenyl)-3-((pyrazolo[1,5-a]pyrimidin-6 ylamino)methyl)benzamide

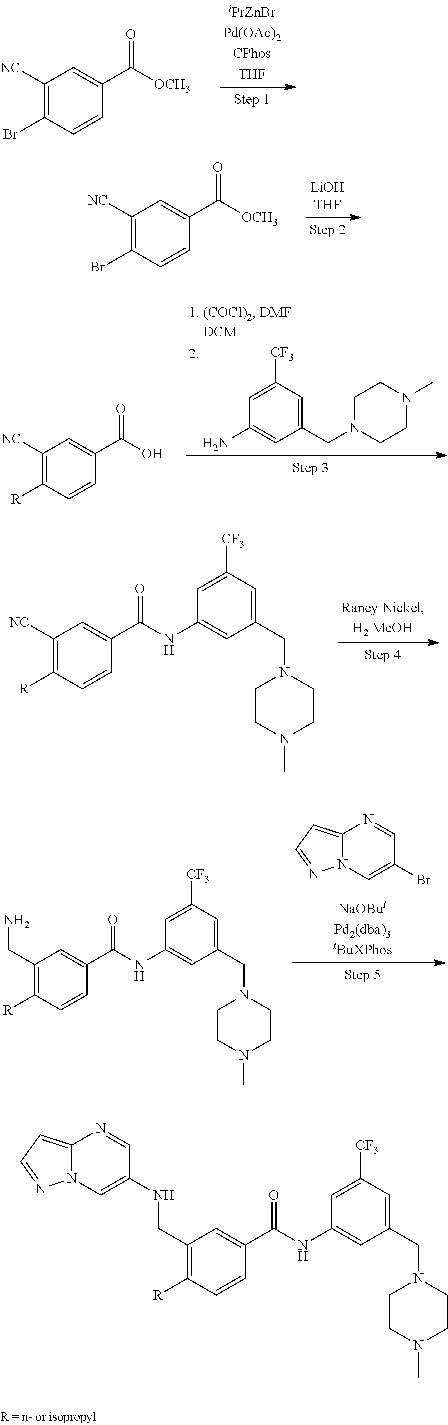

Step 1: Preparation of methyl 3-cyano-4-(propan-2-yl)benzoate; methyl 3-cyano-4-propylbenzoate

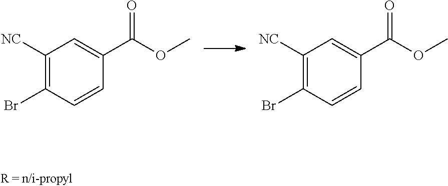

To a mixture of methyl 4-bromo-3-cyanobenzoate (4.00 g, 16.67 mmol), CPhos (0.146 g, 0.333 mmol) and Pd(OAc)₂ (0.037 g, 0.167 mmol) in THF (67 mL), 0.5 M isopropylzinc(II) bromide in THF (40 ml, 20.00 mmol) was added dropwise at 0° C. The reaction was carried out at RT for 3 h, then the reaction mixture was concentrated in vacuo. The residue was partitioned between EtOAc and water, the desired compound was extracted with EtOAc (2×30 mL) and the combined organic phases were washed with water (30 mL), followed by brine (30 mL), then dried with Na₂SO₄ and concentrated in vacuo. The obtained crude product was purified by column chromatography (hexane:EtOAc, from 98:2 to 96:4) to give the isomeric mixture (ratio 1:1) as light yellow oil (2.76 g, 81%).

¹H NMR (300 MHz, DMSO-d6) δ 8.26 (dd, J=3.7, 1.8 Hz, 2H), 8.18 (ddd, J=9.9, 8.2, 1.9 Hz, 2H), 7.73 (d, J=8.3 Hz, 1H), 7.65 (d, J=8.2 Hz, 1H), 3.88 (s, 6H), 3.30 (dd, J=14.1, 7.2 Hz, 1H), 2.84 (dd, J=8.4, 6.8 Hz, 2H), 1.75-1.60 (m, 2H), 1.30 (d, J=6.9 Hz, 6H), 0.93 (t, J=7.3 Hz, 3H).

Step 2: Preparation of 3-cyano-4-(propan-2-yl)benzoic acid; 3-cyano-4-propylbenzoic acid

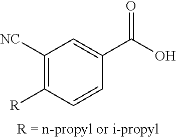

A solution of 1M LiOH (8.61 mL) was added to a mixture of methyl 3-cyano-4-propylbenzoate and methyl-3-cyano-4-isopropylbenzoate (1.75 g, 8.62 mmol, ratio 1:1) in THF (17.5 mL) at 0° C. and the reaction was carried out at RT for 16 h. The reaction mixture was partitioned between EtOAc and water, the aqueous layer was washed with EtOAc (2×30 mL) and the organic phases were discarded. Next, the aqueous layer was acidified with 1M HCl and the product was extracted with EtOAc (3×50 mL). The combined organic layers were dried with Na₂SO₄ and concentrated in vacuo to obtain the titular isomeric mixture as a white solid (ratio 1:1, 1.25 g, 77%).

¹H NMR (300 MHz, DMSO-d6) δiPr 13.40 (s, 1H, OH), 8.26-8.12 (m, 2H, CH), 7.70 (d, J=8.2 Hz, 1H, CH), 3.32-3.21 (m, 1H, CHMe₂), 1.30 (d, J=6.9 Hz, 6H, CH₃) δnPr 13.40 (s, 1H, OH), 8.26-8.12 (m, 2H, CH), 7.62 (d, J=8.1 Hz, 1H, CH), 2.84 (dd, J=8.5, 6.7 Hz, 2H, CH₂), 1.77-1.60 (m, 2H, CH₂), 0.93 (t, J=7.3 Hz, 3H, CH₃).

Step 3: Preparation of 3-cyano-N-{3-[(4-methylpiperazin-1-yl)methyl]-5-(trifluoromethyl)phenyl}-4-(propan-2-yl)benzamide; 3-cyano-N-{3-[(4-methylpiperazin-1-yl)methyl]-5-(trifluoromethyl)phenyl}-4-propylbenzamide

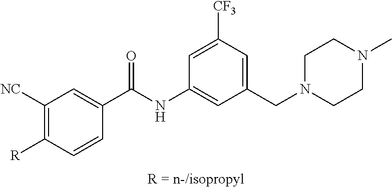

DMF (8.18 μl, 0.106 mmol) and oxalyl chloride (0.370 ml, 4.23 mmol) were added to a solution of 3-cyano-4-propylbenzoic acid and 3-cyano-4-isopropylbenzoic acid (0.40 g, 2.12 mmol, ratio 1:1) in DCM (4 mL) under an argon atmosphere and the mixture was stirred at RT for 4 h. Next, the solvent was removed under reduced pressure and the residue was dissolved in anhydrous DCM (2 mL). The resulting solution was added dropwise to a solution of 3-((4-methylpiperazin-1-yl)methyl)-5-(trifluoromethyl)aniline (0.636 g, 2.325 mmol) and triethylamine (0.589 ml, 4.23 mmol) in anhydrous DCM (2 mL) at 0° C. and stirred at RT for 16 h. The reaction was diluted with DCM, washed with water (25 mL), then the aqueous phase was extracted with DCM (3×25 mL). The combined organic layers were washed with brine (50 mL) and dried with Na₂SO₄ and concentrated in vacuo to give the crude which was purified via column chromatography (DCM:MeOH, from 100:0 to 90:10) to obtain the isomeric mixture as a white solid (ratio 1:1, 0.672 g, 72%).

¹H NMR (300 MHz, DMSO-d6) δiPr 10.63 (s, 1H), 8.41 (m, 1H), 8.28-8.15 (m, 2H), 8.00 (s, 1H), 7.75 (d, J=8.4 Hz), 7.38 (s, 1H), 3.56 (s, 2H), 3.29 (m, 1H), 2.38 (m, 8H), 2.16 (s, 3H), 1.33 (d, J=6.9 Hz, 6H). δnPr 10.63 (s, 1H), 8.41 (m, 1H), 8.28-8.15 (m, 2H), 8.00 (s, 1H), 7.68 (d, J=8.1 Hz, 1H), 7.38 (s, 1H), 3.56 (s, 2H), 2.86 (t, J=7.6 Hz, 2H), 2.38 (m, 8H), 2.16 (s, 3H), 1.69 (p, J=7.4 Hz, 2H), 0.95 (t, J=7.3 Hz, 3H).

Step 4: Preparation of 3-(aminomethyl)-N-{3-[(4-methylpiperazin-1-yl)methyl]-5-(trifluoromethyl)phenyl}-4-(propan-2-yl)benzamide; 3-(aminomethyl)-N-{3-[(4-methylpiperazin-1-yl)methyl]-5-(trifluoromethyl)phenyl}-4-propylbenzamide

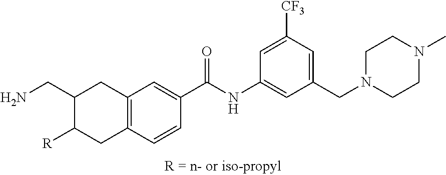

A solution of 3-cyano-N-(3-((4-methylpiperazin-1-yl)methyl)-5-(trifluoromethyl)phenyl)-4-propylbenzamide and 3-cyano-4-isopropyl-N-(3-((4-methylpiperazin-1-yl)methyl)-5-(trifluoromethyl)phenyl)benzamide (0.672 g, 1.51 mmol, ratio 1:1) in MeOH (30 mL) was charged with Raney Nickel (3 mL, 3.02 mmol) and was stirred at RT in the presence of hydrogen atmosphere (balloon) for 3 d. The reaction mixture was filtered through a pad of Celite, concentrated and dried in vacuo to afford to yield the isomeric mixture as a green-yellow solid (ratio iPr: nPr 3:1, 0.589 g, 87%), which was used in the next step without further purification.

¹H NMR (300 MHz, Methanol-d4) δiPr 8.10 (s, 1H), 7.95 (d, J=2.2 Hz, 2H), 7.85 (dd, J=8.1, 2.1 Hz, 1H), 7.50 (d, J=8.2 Hz, 1H), 7.45 (s, 1H), 4.05-3.93 (m, 2H), 3.63 (s, 2H), 2.55 (m, 8H), 2.30 (s, 3H) 1.30 (d, J=6.8 Hz, 6H). CH iPr masked by solvent peak. δnPr 8.10 (s, 1H), 7.95 (d, J=2.2 Hz, 1H), 7.80 (dd, J=8.1, 2.1 Hz, 1H), 7.45 (s, 1H), 7.37 (d, J=8.0 Hz, 2H), 4.05-3.93 (m, 2H), 3.63 (d, J=9.8 Hz, 2H), 2.83-2.68 (m, 2H), 2.55 (m, 8H) 2.30 (s, 3H), 1.69 (m, 2H), 1.04 (t, J=7.3 Hz, 3H).

Step 5: preparation of 4-isopropyl-N-(3-((4-methylpiperazin-1-yl)methyl)-5-(trifluoromethyl)phenyl)-3-((pyrazolo[1,5-a]pyrimidin ylamino)methyl)benzamide (Example 16) and N-(3-((4-methylpiperazin-1-yl)methyl)-5-(trifluoromethyl)phenyl)-4-propyl-3-((pyrazolo[1,5-a]pyrimidin ylamino)methyl)benzamide (Example 17).

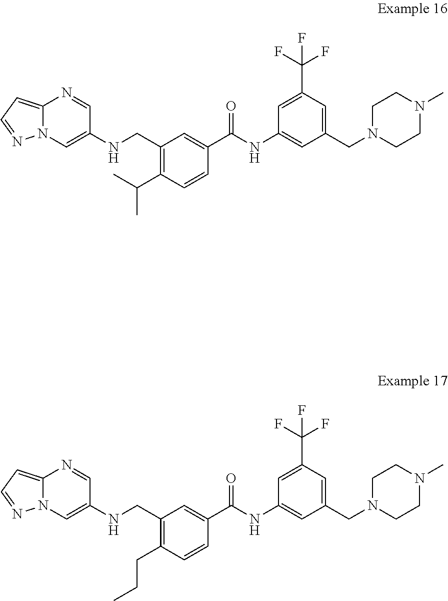 [SMILES: CC(C)c1ccc(C(=O)Nc2cc(CN3CCN(C)CC3)cc(C(F)(F)F)c2)cc1CNc1cnc2ccnn2c1 ; CCCc1ccc(C(=O)Nc2cc(CN3CCN(C)CC3)cc(C(F)(F)F)c2)cc1CNc1cnc2ccnn2c1]

The mixture of 3-(aminomethyl)-4-isopropyl-N-(3-((4-methylpiperazin-1-yl)methyl)-5-(trifluoromethyl)phenyl)benzamide and 3-(aminomethyl)-N-(3-((4-methylpiperazin-1-yl)methyl)-5-(trifluoromethyl)phenyl)-4-propylbenzamide (200 mg, 0.446 mmol, ratio iPr: nPr 3:1) was dissolved in an anhydrous toluene (5 ml). 6-bromopyrazolo[1,5-a]pyrimidine (161 mg, 0.813 mmol) and sodium t-butoxide (65.1 mg, 0.678 mmol) were added, followed by Pd₂(dba)₃ (62.1 mg, 0.068 mmol) and tBuXPhos (57.6 mg, 0.136 mmol). The reaction was stirred at 80° C. for 17 hr, then the reaction mixture was filtered through the Celite. Next, the filtrate was washed with water and the organic phase was concentrated. The crude material was purified via FCC (DCM:MeOH from 1:0 to 0:1), followed by prepHPLC (ACN, H2O+0.1% NH3) yielding 4-isopropyl-N-(3-((4-methylpiperazin-1-yl)methyl)-5-(trifluoromethyl)phenyl)-3-((pyrazolo[1,5-a]pyrimidin-6-ylamino)methyl)benzamide (Example 16) (0.029 g, 15%) and N-(3-((4-methylpiperazin-1-yl)methyl)-5-(trifluoromethyl)phenyl)-4-propyl-3-((pyrazolo[1,5-a]pyrimidin-6-ylamino)methyl)benzamide (Example 17) (0.009 g, 14%) as white solids.

| | t_{R} | Structure and | | Analytical data |
|---|---|---|---|---|
| Ex. | (min) | Chemical Name | Method | NMR |
| Ex. 17 | 2.16 | 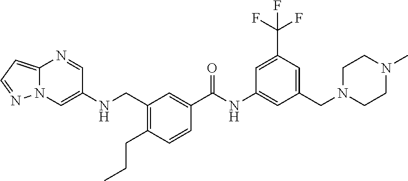 [SMILES: CCCc1ccc(C(=O)Nc2cc(CN3CCN(C)CC3)cc(C(F)(F)F)c2)cc1CNc1cnc2ccnn2c1] | 1 | 1H NMR (300 MHz, Methanol-d4) δ 8.34 (d, J = 2.6 Hz, 1H), 8.07- 8.01 (m, 2H), 7.97-7.94 (m, 1H), 7.89 (s, 1H), 7.87-7.83 (m, 2H), 7.43 (d, J = 8.4 Hz, 1H), 7.41 (s, 1H), 6.54 (dd, J = 2.5, 0.9 Hz, 1H), 4.39 (s, 2H), 3.60 (s, 2H), 2.87-2.79 (m, 2H), 2.52 (s, 8H), 2.28 (s, 3H), 1.74 (dq, |
| | | N-(3-((4-methylpiperazin-1- | | J = 15.0, 7.5 Hz, 2H), 1.04 |
| | | yl)methyl)-5- | | (t, J = 7.3 Hz, 3H). |
| | | (trifluoromethyl)phenyl)-4-propyl- | | |
| | | 3-((pyrazolo[1,5-a]pyrimidin-6- | | |
| | | ylamino)methyl)benzamide | | |
| Ex. 16 | 2.13 | 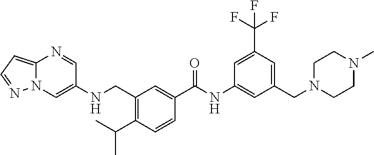 [SMILES: CC(C)c1ccc(C(=O)Nc2cc(CN3CCN(C)CC3)cc(C(F)(F)F)c2)cc1CNc1cnc2ccnn2c1] | 1 | 1H NMR (300 MHz, Methanol-d4) δ 8.33 (d, J = 2.6 Hz, 1H), 8.06 (s, 1H), 8.04-7.99 (m, 2H), 7.91 (dd, J = 8.2, 2.0 Hz, 2H), 7.86 (d, J = 2.5 Hz, 1H), 7.56 (d, J = 8.2 Hz, 1H), 7.41 (s, 1H), 6.54 (dd, J = 2.5, 0.9 Hz, 1H), 4.41 (s, 2H), 3.61 (s, |
| | | 4-isopropyl-N-(3-((4- | | 2H), 3.44-3.36 (m, 1H), |
| | | methylpiperazin-1-yl)methyl)-5- | | 2.52 (s, 8H), 2.28 (s, |
| | | (trifluoromethyl)phenyl)-3- | | 3H), 1.34 (d, J = 6.8 Hz, |
| | | ((pyrazolo[1,5-a]pyrimidin-6- | | 6H). |
| | | ylamino)methyl)benzamide | | |

### Example 18

4-methyl-N-(3-((4-methylpiperazin-1-yl)methyl)-5 (trifluoromethyl)phenyl)-3-((pyrazolo[1,5-a]pyrimidin-6 ylamino)methyl)benzamide

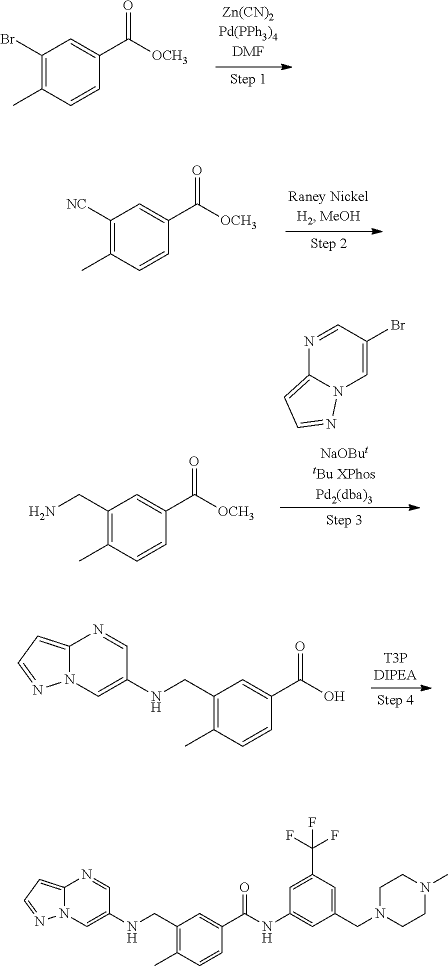 [SMILES: COC(=O)c1ccc(C)c(Br)c1 ; COC(=O)c1ccc(C)c(C#N)c1 ; Brc1cnc2ccnn2c1 ; COC(=O)c1ccc(C)c(CN)c1 ; Cc1ccc(C(=O)O)cc1CNc1cnc2ccnn2c1 ; Cc1ccc(C(=O)Nc2cc(CN3CCN(C)CC3)cc(C(F)(F)F)c2)cc1CNc1cnc2ccnn2c1]

Step 1: Preparation of methyl 3-cyano-4-methylbenzoate

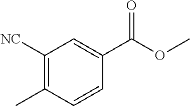

In a 3-necked flask methyl 3-bromo-4-methylbenzoate (10.0 g, 48.7 mmol) and zinc cyanide (6.9 g, 58.5 mmol) were dissolved in anhydrous DMF (150 mL). The solution was degassed under argon. Pd(PPh₃)₄ (2.8 g, 2.4 mmol) was added and the reaction was stirred at 100° C. overnight. After this time the reaction mixture was filtered through a Celite pad and filtrate was concentrated in vacuo. The crude was purified via FCC (hexane:EtOAc, from 98:2 to 95:5) to yield the titular compound as a white solid (7.47 g, 86%).

¹H NMR (300 MHz, CDCl₃) δ 8.30 (d, J=1.7 Hz, 1H), 8.16 (dd, J=1.8, 8.3 Hz, 1H), 7.44 (d, J=8.1 Hz, 1H), 3.97 (s, 3H), 2.64 (s, 3H).

Step 2: Preparation of methyl 3-(aminomethyl)-4-methylbenzoate

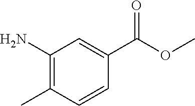

A solution of methyl 3-cyano-4-methylbenzoate (6.99 g, 39.9 mmol) in MeOH (399 mL) was charged with Raney Nickel (80 mL, 50% dispersion in water) and was stirred at RT in the presence of hydrogen atmosphere (balloon) overnight. The reaction mixture was filtered through a pad of Celite, concentrated and dried in vacuo to afford the crude material, which was purified via FCC (hexane:EtOAc, from 50:50 to MeOH:EtOAc:NH₃ 10:89:1) to yield the title compound as a yellow oil (3.63 g, 47%).

¹H NMR (300 MHz, DMSO-d6) δ 8.03 (s, 1H), 7.72 (dd, J=1.9, 7.7 Hz, 1H), 7.27 (d, J=7.7 Hz, 1H), 3.84 (s, 3H), 3.74 (s, 2H), 2.32 (s, 3H), 2.01 (br s, 2H)

Step 3: Preparation of 4-methyl-3-((pyrazolo[1,5-a]pyrimidin-6-ylamino)methyl)benzoic acid

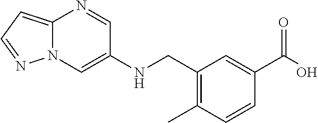 [SMILES: Cc1ccc(C(=O)O)cc1CNc1cnc2ccnn2c1]

To the mixture of 6-bromopyrazolo[1,5-a]pyrimidine (0.398 g, 2.01 mmol) and sodium tert-butoxide (0.483 g, 5.02 mmol), a solution of methyl 3-(aminomethyl)-4-methylbenzoate (0.300 g, 1.67 mmol) in anhydrous toluene (6 mL) was added. The mixture was degassed under argon, then tBu XPhos (0.142 g, 0.335 mmol) and Pd₂(dba)₃ (0.153 g, 0.167 mmol) were added. The reaction was carried out at 110° C. overnight, then filtered through a pad of Celite, concentrated and dried in vacuo to afford the crude material, which was purified by FCC (MeOH:DCM, from 5:95 to 20:80) to give the titular material as a red solid (0.059 g, 11%)

¹H NMR (300 MHz, DMSO-d6) δ 12.43 (s, 1H), 8.41 (s, 1H), 7.96-7.84 (m, 2H), 7.78 (d, J=8.1 Hz, 1H), 7.35 (d, J=7.8 Hz, 2H), 6.54 (s, 1H), 6.41 (t, J=5.6 Hz, 1H), 4.30 (d, J=5.4 Hz, 2H), 2.43 (s, 3H).

Step 4: Preparation of 4-methyl-N-(3-((4-methylpiperazin-1-yl)methyl)-5-(trifluoromethyl)phenyl)-3-((pyrazolo[1,5-a]pyrimidin-6-ylamino)methyl)benzamide (Example 18)

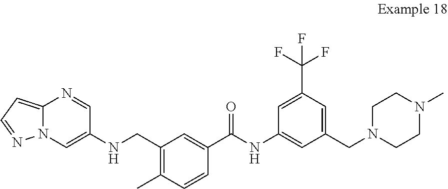 [SMILES: Cc1ccc(C(=O)Nc2cc(CN3CCN(C)CC3)cc(C(F)(F)F)c2)cc1CNc1cnc2ccnn2c1]

Preparation of Example 18 was performed according to the General method A for amide coupling, reacting 4-methyl-3-((pyrazolo[1,5-a]pyrimidin-6-ylamino)methyl)benzoic acid (0.03 g) with the required amine to give a yellow solid (0.006 g; 11%).

| | Method | | t_{R} | |
|---|---|---|---|---|
| Ex. | purification | Method | (min) | Analytical data NMR |
| Ex. 18 | pTLC | 5 | 5.53 | 1H NMR (300 MHz, Methanol- |
| | | | | d4) δ 8.34 (d, J = 2.6 Hz, 1H), |
| | | | | 8.04 (s, 1H), 8.01 (d, J = 2.0 |
| | | | | Hz, 1H), 7.95 (dd, J = 2.7, 0.9 |
| | | | | Hz, 1H), 7.88 (s, 1H), 7.85 (d, |
| | | | | J = 2.5 Hz, 1H), 7.82 (dd, J = |
| | | | | 7.9, 2.0 Hz, 1H), 7.44-7.36 |
| | | | | (m, 2H), 6.53 (dd, J = 2.6, 0.8 |
| | | | | Hz, 1H), 4.36 (s, 2H), 3.60 (s, |
| | | | | 2H), 2.51 (m, 11H), 2.27 |
| | | | | (s, 3H). |

### Example 19

4-methyl-N-(3-((4-methylpiperazin-1-yl)methyl) (trifluoromethyl)phenyl)-3-((pyrimidin-5-ylamino)methyl)benzamide

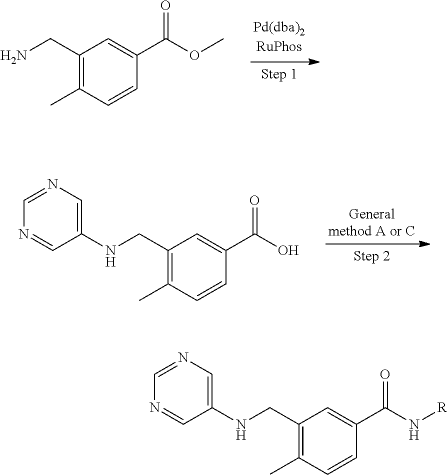

Step 1: Preparation of 4-methyl-3-((pyrimidin-5-ylamino)methyl)benzoic acid

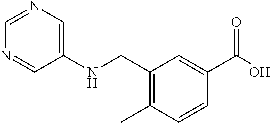 [SMILES: Cc1ccc(C(=O)O)cc1CNc1cncnc1]

Methyl 3-(aminomethyl)-4-methylbenzoate) prepared as described in Example 18, step 1-3 (0.500 g, 2.79 mmol, Cs₂CO₃ (2.73 g, 8.37 mmol) and 5-bromopyrimidine (1.06 g, 6.67 mmol) were suspended in anhydrous toluene (9.0 mL). The suspension was degassed, then RuPhos (0.520 g, 1.11 mmol) and Pd(dba)₂ (0.320 g, 0.557 mmol) were added and the reaction was carried out at 100° C. for 24 h. The reaction mixture was filtered through a pad of celite, concentrated and dried in vacuo to afford the crude material, which was purified via column chromatography (DCM:3.5M NH₃ in MeOH, from 80:20 to 50:50) to yield the titular compound as a yellow oil (0.755 g, 100%).

¹H NMR (300 MHz, DMSO-d6) δ 8.64 (s, 1H), 8.39 (s, 1H), 8.13 (s, 2H), 7.76-7.67 (m, 2H), 7.25 (dd, J=7.7, 5.1 Hz, 2H), 4.33 (d, J=5.7 Hz, 2H), 2.37 (d, J=2.3 Hz, 3H).

Step 2: preparation of 4-methyl-N-(3-((4-methylpiperazin-1-yl)methyl)-5-(trifluoromethyl)phenyl)-3-((pyrimidin-5-ylamino)methyl)benzamide (Example 19), 4-methyl-N-(3-(4-methyl-1H-imidazol-1-yl)-5-(trifluoromethyl)phenyl)-3-((pyrimidin-5-ylamino)methyl)benzamide (Example 20), N-(4-methoxy-3-(trifluoromethyl)phenyl)-4-methyl-3-((pyrimidin-5-ylamino)methyl)benzamide (Example 31) and 4-methyl-3-((pyrimidin-5-ylamino)methyl)-N-(3-(trifluoromethyl)phenyl)benzamide (Example 32).

Preparations of Example 19 and Example 20 were performed according to the General method A while preparations of Example 31 and Example 32 were performed according to the General method C, reacting 4-methyl-3-((pyrimidin ylamino)methyl)benzoic acid with the required amine to give the following compounds:

| | | | Amount of | | | |
|---|---|---|---|---|---|---|
| | Structure and | Amount | product | | t_{R} | Analytical data |
| Ex. | chemical name | of SM | (yield) | Method | (min) | NMR |
| Ex. 19 | 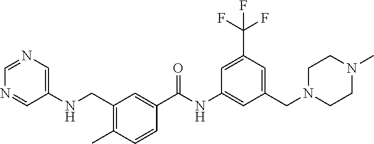 [SMILES: Cc1ccc(C(=O)Nc2cc(CN3CCN(C)CC3)cc(C(F)(F)F)c2)cc1CNc1cncnc1] | 0.05 g | 0.003 g (3%) | 3 | 3.38 | ¹H NMR (300 MHz, Methanol-d4) δ 8.45 (s, 1H), 8.40 (s, 1H), 8.16 (s, 2H), 8.00 (s, 1H), 7.94 (d, J = 2.2 Hz, 2H), 7.82 |
| | 4-methyl-N-(3-((4- | | | | | (dd, J = 7.9, 2.0 |
| | methylpiperazin-1- | | | | | Hz, 1H), 7.45- |
| | yl)methyl)-5- | | | | | 7.37 (m, 2H), |
| | (trifluoromethyl)phenyl)- | | | | | 4.44 (s, 2H), |
| | 3-((pyrimidin-5- | | | | | 3.68 (s, 2H), |
| | ylamino)methyl)benzamide | | | | | 3.06 (s, 4H), |
| | | | | | | 2.85-2.58 (m, |
| | | | | | | 7H), 2.48 (s, |
| | | | | | | 3H). |
| Ex. 20 | 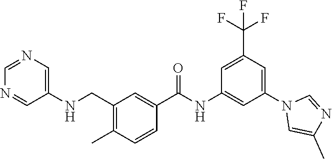 [SMILES: Cc1cn(-c2cc(NC(=O)c3ccc(C)c(CNc4cncnc4)c3)cc(C(F)(F)F)c2)cn1] | 0.05 g | 0.006 g (6%) | 5 | 6.333 | ¹H NMR (400 MHz, DMSO- d6) δ 10.61 (s, 1H), 8.42 (s, 1H), 8.29 (t, 1H), 8.20 (d, J = 1.4 Hz, 1H), 8.17 (s, 2H), 8.12 (s, 1H), 7.93 (d, J = 1.9 |
| | 4-methyl-N-(3-(4-methyl- | | | | | Hz, 1H), 7.89 |
| | 1H-imidazol-1-yl)-5- | | | | | (dd, J = 7.8, 2.0 |
| | (trifluoromethyl)phenyl)- | | | | | Hz, 1H), 7.72 |
| | 3-((pyrimidin-5- | | | | | (s, 1H), 7.48 (t, |
| | ylamino)methyl)benzamide | | | | | J = 1.3 Hz, 1H), |
| | | | | | | 7.43 (d, J = 7.9 |
| | | | | | | Hz, 1H), 6.66 |
| | | | | | | (t, J = 5.7 Hz, |
| | | | | | | 1H), 4.39 (d, |
| | | | | | | J = 5.6 Hz, 2H), |
| | | | | | | 2.44 (s, 3H), |
| | | | | | | 2.18 (d, J = 1.0 |
| | | | | | | Hz, 3H). |
| Ex. 31 | 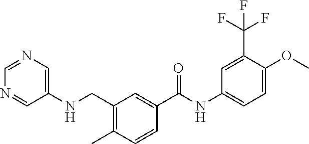 [SMILES: COc1ccc(NC(=O)c2ccc(C)c(CNc3cncnc3)c2)cc1C(F)(F)F] | 0.10 g | 0.069 g (41%) | 6 | 2.553 | ¹H NMR (300 MHz, Methanol-d4) δ 8.40 (s, 1H), 8.16 (s, 2H), 7.93 (d, J = 2.3 Hz, 2H), 7.86 (dd, J = 9.0, 2.7 Hz, 1H), 7.80 |
| | N-(4-methoxy-3- | | | | | (dd, J = 7.9, 2.0 |
| | (trifluoromethyl)phenyl)- | | | | | Hz, 1H), 7.38 (d, |
| | 4-methyl-3-((pyrimidin-5- | | | | | J = 7.9 Hz, 1H), |
| | ylamino)methyl)benzamide | | | | | 7.17 (d, J = 9.0 |
| | | | | | | Hz, 1H), 4.43 (s, |
| | | | | | | 2H), 3.90 (s, |
| | | | | | | 3H), 2.47 (s, |
| | | | | | | 3H). |
| Ex. 32 | 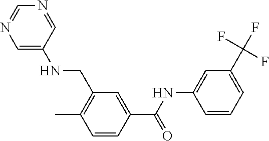 [SMILES: Cc1ccc(C(=O)Nc2cccc(C(F)(F)F)c2)cc1CNc1cncnc1] | 0.10 g | 0.146 g (36%) | 6 | 2.693 | ¹H NMR (300 MHz, Methanol-d4) δ 8.40 (s, 1H), 8.17 (s, 2H), 8.12 (s, 1H), 7.95 (d, J = 2.0 Hz, 1H), 7.91 (d, J = 8.2 Hz, 1H), |
| | 4-methyl-3-((pyrimidin-5- | | | | | 7.82 (dd, J = 7.8, |
| | ylamino)methyl)-N-(3- | | | | | 2.0 Hz, 1H), |
| | (trifluoromethyl)phenyl) | | | | | 7.54 (t, J = 8.0 |
| | benzamide | | | | | Hz, 1H), 7.41 (t, |
| | | | | | | J = 7.1 Hz, 2H), |
| | | | | | | 4.44 (s, 2H), |
| | | | | | | 2.48 (s, 3H). |

### Example 21

N-(2-methyl-5-((3-((4-methylpiperazin-1-yl)methyl) (trifluoromethyl)phenyl)carbamoyl)benzyl)imidazo[1,2-a]pyridine carboxamide

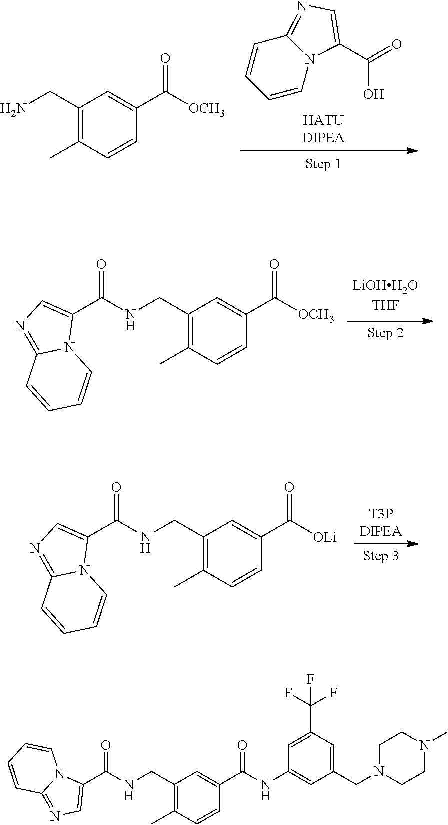

Step 1: Preparation of methyl 3-((imidazo[1,2-a]pyridine-3-carboxamido)methyl)-4-methylbenzoate

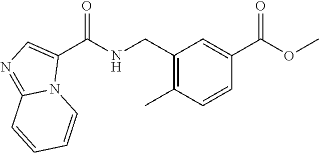 [SMILES: COC(=O)c1ccc(C)c(CNC(=O)c2cnc3ccccn23)c1]

To a mixture of methyl 3-(aminomethyl)-4-methylbenzoate (0.7 g, 3.91 mmol), prepared as described in Example 18, step 1-2, imidazo[1,2-a]pyridine-3-carboxylic acid (0.760 g, 4.69 mmol) and HATU (1.49 g, 3.91 mmol, 1 eq), anhydrous DCM (13 mL) was added, followed by DIPEA (1.4 mL, 7.81 mmol). The reaction was stirred overnight at RT, then the reaction mixture was partitioned between DCM (15 mL) and water (15 mL) and the desired compound extracted with DCM (2×15 mL). The combined organic phases were washed with water (15 mL), brine (15 mL), then dried with Na₂SO₄, filtered and concentrated. The crude material was purified via FCC (MeOH:DCM, from 1:99 to 5:95) to yield the title compound (1.26 g, 66%) as an off-white solid.

¹H NMR (300 MHz, CDCl₃) δ 9.69 (d, J=6.7 Hz, 1H, CH), 8.26 (br s, 1H, CH), 8.03 (s, 1H, CH), 7.90 (dd, J=7.9, 1.6 Hz, 1H, CH), 7.76 (d, J=8.9 Hz, 1H, CH), 7.46 (t, J=7.6 Hz, 1H, CH), 7.31 (s, 1H, CH), 7.07 (t, J=6.9 Hz, 1H, CH), 6.93 (br s, 1H, NH), 4.72 (d, J=5.5 Hz, 2H, CH₂), 3.90 (s, 3H, CH₃), 2.48 (s, 3H, CH₃).

Step 2: Preparation of Lithium 3-((imidazo[1,2-a]pyridine-3-carboxamido)methyl)-4-methylbenzoate

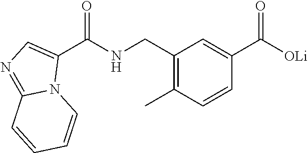

Methyl 3-((imidazo[1,2-a]pyridine-3-carboxamido)methyl)-4-methylbenzoate

(0.416 g, 1.29 mmol) was dissolved in THF (13 mL) and 1 M LiOH solution (2 mL, 1.93 mmol) was added. The reaction was stirred for 16 h at 60° C. The solvent was removed in vacuo and the crude material triturated with diethyl ether (30 mL) and filtered. The solid was collected to give the titular product as an off-white solid (0.348 g, 86%).

¹H NMR (300 MHz, DMSO-d6) δ9.54 (d, J=7.0 Hz, 1H), 9.02 (br s, 1H), 8.45 (s, 1H), 7.80 (s, 1H), 7.69 (m, 1H), 7.69 (m, 1H), 7.45 (ddd J=8.7, 6.8, 1.3 Hz, 1H), 7.10 (m, 1H), 7.10 (m, 1H), 4.49 (s, 2H), 2.32 (s, 3H).

Step 3: Preparation of N-(2-methyl-5-((3-((4-methylpiperazin-1-yl)methyl)-5-(trifluoromethyl)phenyl)carbamoyl)benzyl)imidazo[1,2-a]pyridine-3-carboxamide (Example 21) and N-(2-methyl-5-((3-(4-methyl-1H-imidazol-1-yl)-5-(trifluoromethyl)phenyl)carbamoyl)benzyl)imidazo[1,2-a]pyridine-3-carboxamide (Example 22).

Preparations of Example 21 and Example 22 were performed according to the General method A for amide coupling, reacting 4 Lithium 3-((imidazo[1,2-a]pyridine carboxamido)methyl)-4-methylbenzoate with the required amines to give the following compounds:

| | | | Amount of | | | |
|---|---|---|---|---|---|---|
| | Structure and | Amount | product | | t_{R} | |
| Ex. | chemical name | of SM | (yield) | Method | (min) | Analytical data NMR |
| Ex.21 | 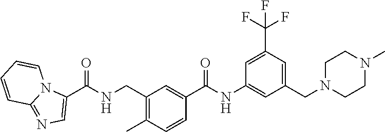 [SMILES: Cc1ccc(C(=O)Nc2cc(CN3CCN(C)CC3)cc(C(F)(F)F)c2)cc1CNC(=O)c1cnc2ccccn12] | 0.05 g | 0.025 g (27%) | 5 | 4.81 | 1H NMR (300 MHz, Methanol-d4) δ 9.51 (dt, J = 7.0, 1.2 Hz, 1H), 8.31 (s, 1H), 8.03 (s, 1H), 7.96 (d, J = 1.9 Hz, 1H), 7.88 (s, 1H), 7.79 (dd, J = 7.9, 2.0 Hz, 1H), 7.69 (dt, J = 9.1, |
| | N-(2-methyl-5-((3- | | | | | 1.2 Hz, 1H), 7.52 (ddd, |
| | ((4- | | | | | J = 9.0, 6.8, 1.3 Hz, |
| | methylpiperazin-1- | | | | | 1H), 7.40 (s, 1H), 7.37 |
| | yl)methyl)-5- | | | | | (d, J = 7.9 Hz, 1H), 7.16- |
| | (trifluoromethyl) | | | | | 7.10 (m, 1H), 4.69 (s, |
| | phenyl)carbamoyl) | | | | | 2H), 3.60 (s, 2H), 2.50 |
| | benzyl)imidazo[1,2- | | | | | (s, 11H), 2.28 (s, 3H). |
| | a]pyridine-3- | | | | | |
| | carboxamide | | | | | |
| Ex.22 | 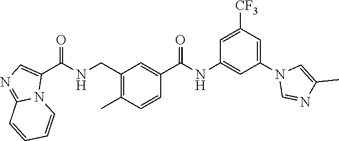 [SMILES: Cc1cn(-c2cc(NC(=O)c3ccc(C)c(CNC(=O)c4cnc5ccccn45)c3)cc(C(F)(F)F)c2)cn1] | 0.05 g | 0.037 g (43%) | 3 | 3.30 | 1H NMR (300 MHz, Methanol-d4) δ 9.55- 9.47 (m, 1H), 8.31 (s, 1H), 8.21 (t, J = 2.0 Hz, 1H), 8.10 (s, 1H), 8.05 (s, 1H), 7.99 (d, J = 1.9 Hz, 1H), 7.82 (dd, J = 7.8, 2.0 Hz, 1H), 7.72- 7.64 (m, 1H), 7.59 (s, |
| | N-(2-methyl-5-((3- | | | | | 1H), 7.52 (ddd, J = 8.9, |
| | (4-methyl-1H- | | | | | 6.9, 1.3 Hz, 1H), 7.42- |
| | imidazol-1-yl)-5- | | | | | 7.31 (m, 2H), 7.12 (td, |
| | (trifluoromethyl) | | | | | J = 6.9, 1.2 Hz, 1H), 4.69 |
| | phenyl)carbamoyl) | | | | | (s, 2H), 2.50 (s, 3H), |
| | benzyl)imidazo[1,2- | | | | | 2.26 (d, J = 1.0 Hz, 3H). |
| | a]pyridine-3- | | | | | |
| | carboxamide | | | | | |

### Example 23

N-methyl-4-((2-methyl-5-((3-(4-methyl-1H-imidazol-1-yl)-5-(trifluoromethyl)phenyl)carbamoyl)benzyl)amino)picolinamide

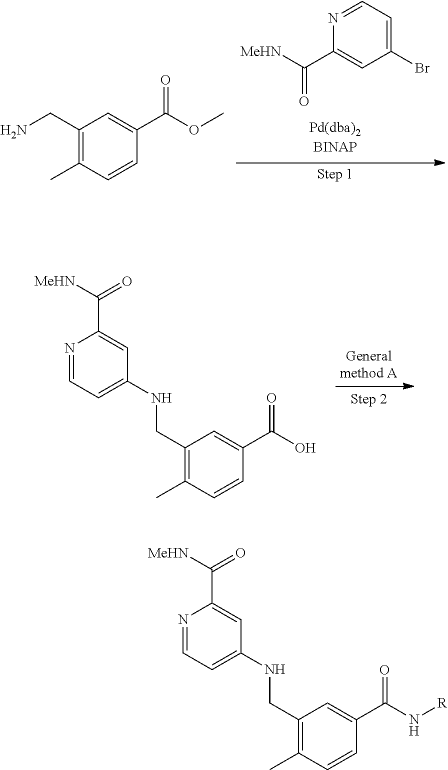

Step 1: Preparation of 4-methyl-3-(((2-(methylcarbamoyl)pyridine-4-yl)amino)methyl)benzoic acid

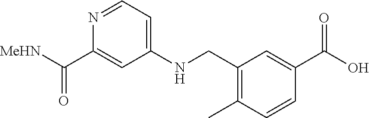 [SMILES: CNC(=O)c1cc(NCc2cc(C(=O)O)ccc2C)ccn1]

To 4-bromo-N-methylpyridine-2-carboxamide (0.720 g, 3.35 mmol), prepared as described in Example 18, step 1-2, and Cs₂CO₃ (2.73 g, 8.37 mmol), a solution of methyl-3-(aminomethyl)-4-methyl-benzoate (0.500 g, 2.79 mmol) in anhydrous toluene (9 mL) was added. The reaction mixture was degassed and BINAP (0.347 g, 0.558 mmol) and Pd(dba)₂ (0.160 g, 0.279 mmol) were added and the reaction stirred at 100° C. overnight. The reaction mixture was filtered through a pad of Celite, concentrated and dried in vacuo to afford the crude material, which was purified via FCC (MeOH:DCM, from 10:90 to 50:50) to obtain the titular product as a yellow solid (0.650 g, 78%).

¹H NMR (300 MHz, DMSO-d6) δ8.56 (q, J=4.6 Hz, 1H), 8.06 (d, J=5.7 Hz, 1H), 7.82-7.78 (m, 2H), 7.78-7.72 (m, 1H), 7.39 (t, J=5.6 Hz, 1H), 7.33 (d, J=7.9 Hz, 1H), 7.24 (s, 1H), 6.63 (d, J=5.4 Hz, 1H), 4.38 (d, J=5.5 Hz, 2H), 2.76 (d, J=4.9 Hz, 3H), 2.40 (s, 3H).

Step 2: Preparation of N-methyl-4-((2-methyl-5-((3-(4-methyl-1H-imidazol-1-yl)-5-(trifluoromethyl)phenyl)carbamoyl)benzyl)amino)picolinamide (Example 23) and N-methyl-4-((2-methyl-5-((3-((4-methylpiperazin-1-yl)methyl)-5-(trifluoromethyl)phenyl)carbamoyl)benzyl)amino)picolinamide (Example 24)

Preparations of Example 23 and Example 24 were performed according to the General method A for amide coupling, reacting 4-methyl-3-(((2-(methylcarbamoyl)pyridine-4-yl)amino)methyl)benzoic acid with the required amines to give the following compounds.

| | | | Amount of | | | |
|---|---|---|---|---|---|---|
| | Structure and | Amount | product | | t_{R} | |
| Ex. | chemical name | of SM | (yield) | Method | (min) | Analytical data NMR |
| Ex. 23 | 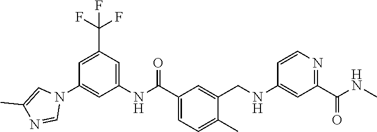 | 0.04 g | 0.017 g (24%) | 5 | 4.187 | 1H NMR (300 MHz, DMSO-d6) δ 10.61 (s, 1H), 8.57 (d, J = 4.9 Hz, 1H), 8.29 (d, J = 2.0 Hz, 1H), 8.20 (d, J = 1.4 Hz, 1H), 8.14-8.04 (m, 2H), 7.89 (m, 2H), 7.70 (s, 1H), 7.48 (t, J = 1.3 |
| | N-methyl-4-((2- | | | | | Hz, 1H), 7.45-7.34 |
| | methyl-5-((3-(4- | | | | | (m, 2H), 7.27 (s, 1H), |
| | methyl-1H- | | | | | 6.72-6.62 (m, 1H), |
| | imidazol-1-yl)-5- | | | | | 4.42 (d, J = 5.0 Hz, 2H), |
| | (trifluoromethyl) | | | | | 2.80-2.73 (m, 3H), |
| | phenyl)carbamoyl) | | | | | 2.43 (s, 3H), 2.18 (d, J = |
| | benzyl)amino) | | | | | 1.0 Hz, 3H). |
| | picolinamide | | | | | |
| Ex. 24 | 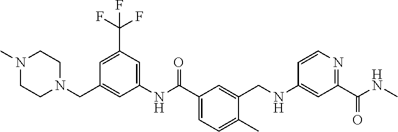 | 0.04 g | 0.013 g (17%) | 5 | 4.160 | 1H NMR (300 MHz, DMSO-d6) δ 10.45 (s, 1H), 8.57 (d, J = 5.0 Hz, 1H), 8.16 (s, 1H), 8.08 (d, J = 5.7 Hz, 1H), 7.99 (s, 1H), 7.87 (d, J = 6.6 Hz, 2H), 7.43-7.32 (m, 3H), 7.27 (s, 1H), |
| | N-methyl-4-((2- | | | | | 6.68 (dd, J = 5.8, 2.3 |
| | methyl-5-((3-((4- | | | | | Hz, 1H), 4.41 (d, J = 5.4 |
| | methylpiperazin-1- | | | | | Hz, 2H), 3.53 (s, 2H), |
| | yl)methyl)-5- | | | | | 2.77 (d, J = 4.9 Hz, 3H), |
| | (trifluoromethyl) | | | | | 2.43 (s, 3H), 2.36 (d, J = |
| | phenyl)carbamoyl) | | | | | 18.8 Hz, 8H), 2.16 (s, |
| | benzyl)amino)picolinamide | | | | | 3H). |

### Example 25

N-(2-methyl-5-((3-(4-methyl-1H-imidazol-1-yl)-5-(trifluoromethyl)phenyl)carbamoyl)benzyl)-1H-pyrrolo[2,3-b]pyridine-5-carboxamide

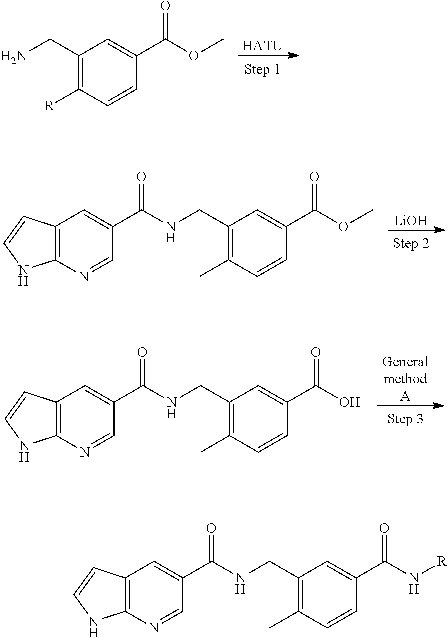

Step 1: Preparation of methyl 4-methyl-3-(((1H-pyrrolo[2,3-b]pyridin-5-yl)formamido)methyl)benzoate

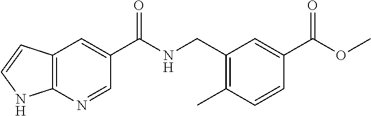 [SMILES: COC(=O)c1ccc(C)c(CNC(=O)c2cnc3[nH]ccc3c2)c1]

To a solution of methyl 3-(aminomethyl)-4-methylbenzoate (0.300 g, 1.67 mmol), prepared as described in Example 18, step 1-2, 1H-pyrrolo[2,3-b]pyridine-5-carboxylic acid (0.324 g, 2.01 mmol) and HATU (0.636 g, 1.67 mmol) in anhydrous DCM (5 mL), DIPEA (0.6 mL, 3.35 mmol) was added, then the mixture was stirred 16 h at RT. The reaction was quenched by the addition of saturated NaHCO₃, then extracted with DCM (×3). The organic layers were dried over Na₂SO₄ and evaporated to dryness. The crude product was purified by column chromatography (DCM:MeOH, from 98:2 to 96:4) to obtain the titular amide as a yellow-white solid (0.323 g. 60%).

NMR (d-DMSO, 300 MHz): δ 11.93 (s, 1H), 9.04 (t, J=5.7 Hz, 1H), 8.78 (d, J=2.1 Hz, 1H), 8.50 (d, J=2.0 Hz, 1H), 7.91 (d, J=1.5 Hz, 1H), 7.77 (dd, J=7.8, 1.8 Hz, 1H), 7.58 (dd, J=3.5, 2.3 Hz, 1H), 7.34 (d, J=7.9 Hz, 1H), 6.63-6.55 (m, 1H), 4.54 (d, J=5.7 Hz, 2H), 3.80 (s, 3H), 2.43 (s, 3H).

Step 2: Preparation of lithium 3-((1H-pyrrolo[2,3-b]pyridine-5-carboxamido)methyl)-4-methylbenzoate

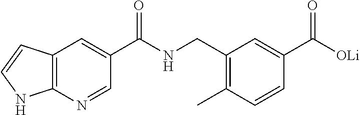

Methyl 4-methyl-3-(((1H-pyrrolo[2,3-b]pyridin-5-yl)formamido)methyl)benzoate (323 mg, 0.999 mmol) was dissolved in THF (10 mL) then 1 M LiOH aqueous solution (3.0 mL) was added and the reaction mixture was stirred for 3 days at RT. Next, the reaction mixture was concentrated in vacuo and the residue triturated with diethyl ether to obtain the titular compound as a bright yellow solid (238 mg, 76%).

NMR (d-DMSO, 300 MHz): δ 8.58 (d, J=5.7 Hz, 1H), 8.55 (d, J=2.3 Hz, 1H), 8.25 (d, J=2.3 Hz, 1H), 7.86 (d, J=1.6 Hz, 1H), 7.66 (dd, J=7.6, 1.7 Hz, 1H), 7.47 (d, J=2.6 Hz, 1H), 7.05 (d, J=7.7 Hz, 1H), 6.25 (d, J=2.6 Hz, 1H), 4.45 (d, J=5.3 Hz, 2H), 2.32 (s, 3H).

Step 3: Preparation of N-(2-methyl-5-((3-(4-methyl-1H-imidazol-1-yl)-5-(trifluoromethyl)phenyl)carbamoyl)benzyl)-1H-pyrrolo[2,3-b]pyridine-5-carboxamide (Example 25) and N-(2-methyl-5-((3-((4-methylpiperazin-1-yl)methyl)-5-(trifluoromethyl)phenyl)carbamoyl)benzyl)-1H-pyrrolo[2,3-b]pyridine-5-carboxamide (Example 26)

Preparations of Example 25 and Example 26 were performed according to the General method A for amide coupling, reacting lithium 3-((1H-pyrrolo[2,3-b]pyridine-5-carboxamido)methyl)-4-methylbenzoate with the required amines to give the following compounds:

| | | | Amount of | | | |
|---|---|---|---|---|---|---|
| | Structure and | Amount | product | | t_{R} | Analytical data |
| Ex. | chemical name | of SM | (yield) | Method | (min) | NMR |
| Ex. 25 | 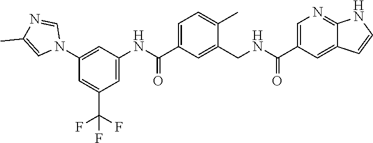 [SMILES: Cc1cn(-c2cc(NC(=O)c3ccc(C)c(CNC(=O)c4cnc5[nH]ccc5c4)c3)cc(C(F)(F)F)c2)cn1] | 0.04 g | 0.007 g (10%) | 5 | 5.417 | 1H NMR (300 MHz, DMSO-d6) δ 11.96 (s, 1H), 10.85 (s, 1H), 9.13 (s, 1H), 8.80 (d, J = 2.1 Hz, 1H), 8.53 (d, J = 2.1 Hz, 1H), 8.30 (s, 1H), 8.19 (d, J = 1.4 Hz, 1H), 8.14 (s, |
| | N-(2-methyl-5-((3- | | | | | 1H), 8.00 (s, 1H), |
| | (4-methyl-1H- | | | | | 7.93-7.85 (m, 1H), |
| | imidazol-1-yl)-5- | | | | | 7.66 (s, 1H), 7.57 (d, |
| | (trifluoromethyl) | | | | | J = 3.5 Hz, 1H), 7.47 |
| | phenyl)carbamoyl) | | | | | (s, 1H), 7.38 (d, J = |
| | benzyl)-1H- | | | | | 7.9 Hz, 1H), 6.57 (d, |
| | pyrrolo[2,3- | | | | | J = 3.5 Hz, 1H), 4.58 |
| | b]pyridine-5- | | | | | (d, J = 5.5 Hz, 2H), |
| | carboxamide | | | | | 2.45 (s, 3H), 2.17 (s, |
| | | | | | | 3H) |
| Ex. 26 | 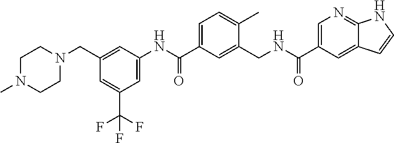 [SMILES: Cc1ccc(C(=O)Nc2cc(CN3CCN(C)CC3)cc(C(F)(F)F)c2)cc1CNC(=O)c1cnc2[nH]ccc2c1] | 0.04 g | 0.0025 g (3%) | 5 | 5.310 | 1H NMR (300 MHz, Methanol-d4) δ 8.78 (s, 1H), 8.54 (d, J = 2.1 Hz, 1H), 8.07 (s, 1H), 7.97 (s, 1H), 7.90 (s, 1H), 7.81 (d, J = 8.0 Hz, 1H), 7.50 (d, J = 3.5 Hz, 1H), 7.46-7.35 (m, 2H), |
| | N-(2-methyl-5- | | | | | 6.63 (d, J = 3.5 Hz, |
| | ((3-((4- | | | | | 1H), 4.72 (s, 2H), |
| | methylpiperazin- | | | | | 3.62 (s, 2H), 2.53 (s, |
| | 1-yl)methyl)-5- | | | | | 11H), 2.29 (s, 3H). |
| | (trifluoromethyl) | | | | | |
| | phenyl)carbamoyl) | | | | | |
| | benzyl)-1H- | | | | | |
| | pyrrolo[2,3- | | | | | |
| | b]pyridine-5- | | | | | |
| | carboxamide | | | | | |

### Example 27

N-(2-isopropyl-5-((3-((4-methylpiperazin-1-yl)methyl) (trifluoromethyl)phenyl)carbamoyl)benzyl)-1H-pyrrolo[2,3-b]pyridine carboxamide

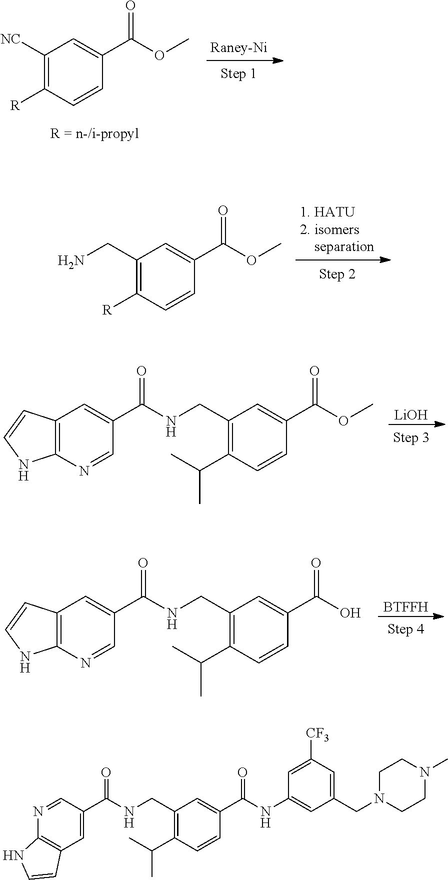

Step 1: Preparation of methyl 3-(aminomethyl)-4-isopropylbenzoate

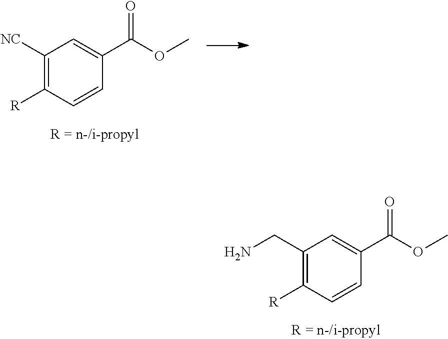

To the mixture of methyl 3-cyano-4-isopropylbenzoate and methyl 3-cyano-4-propylbenzoate (2.76 g, 13.6 mmol, ratio 1:1), prepared as described in Example 16, step 1-2, in MeOH (200 mL), Raney Nickel (4 ml, 6.80 mmol) was added. The reaction mixture was placed in the Parr apparatus under hydrogen atmosphere (7 atm.). The reaction was carried out at RT for 60 h, then it was filtered through a pad of Celite, concentrated and dried in vacuo to afford the crude, which was purified via column chromatography (DCM:5.5 M NH₃ in MeOH, from 99:1 to 90:10)) to obtain the isomeric mixture (isomeric ratio 1:1, 1.33 g, 94%) as a light yellow oil.

¹H NMR (d-DMSO, 300 MHz): iPr δ 7.77 (ddd, J=15.1, 8.0, 2.0 Hz, 2H), 3.83 (s, 3H), 3.82 (s, 2H), 3.25 (sept, J=6.8 Hz, 1H), 2.07 (br, 2H), 1.20 (d, J=6.8 Hz, 6H) nPr δ 8.04 (dd, J=11.3, 1.9 Hz, 2H), 7.27 (d, J=7.9 Hz, 1H), 3.83 (s, 3H), 3.78 (s, 2H), 2.71-2.59 (m, 2H), 2.07 (s, 2H), 1.57 (sext, J=7.3 Hz, 2H), 0.94 (t, J=7.3 Hz, 3H).

Step 2: Preparation of methyl 3-((1H-pyrrolo[2,3-b]pyridine carboxamido)methyl)-4-isopropylbenzoate

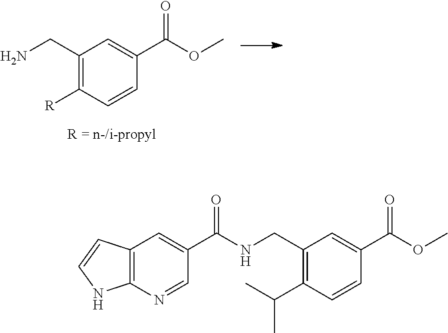

To a mixture of 1H-pyrrolo[2,3-b]pyridine-5-carboxylic acid (250 mg, 1.54 mmol) and HATU (488 mg, 1.283 mmol), a solution of methyl 3-(aminomethyl)-4-isopropylbenzoate and methyl 3-(aminomethyl)-4-propylbenzoate (266 mg, 1.28 mmol, ratio 1:1) in DCM (4.3 mL) was added, followed by addition of DIPEA (448 μl, 2.57 mmol). The reaction was carried out at RT for 16 h, then it was diluted with DCM, washed with water and the desired compound re-extracted with DCM. The combined organic phases were concentrated and dried in vacuo to afford the crude, which was purified via FCC (hexane:EtOAc, from 100:0 to 50:50) to afford the isomeric mixture. Next, the isomers were submitted for preparative HPLC separation, to yield the desired isomer (105 mg, 47%) as a white crystalline solid.

1H NMR (300 MHz, DMSO-d6) δ 11.95 (s, 1H), 9.06 (t, J=5.7 Hz, 1H), 8.77 (d, J=2.1 Hz, 1H), 8.49 (d, J=2.1 Hz, 1H), 7.95 (d, J=1.9 Hz, 1H), 7.85 (dd, J=8.1, 1.9 Hz, 1H), 7.58 (d, J=3.4 Hz, 1H), 7.49 (d, J=8.1 Hz, 1H), 6.58 (d, J=3.5 Hz, 1H), 4.62 (d, J=5.6 Hz, 2H), 3.82 (s, 3H), 3.38 (s, 1H), 1.24 (d, J=6.8 Hz, 6H).

Step 3: Preparation of 3-((1H-pyrrolo[2,3-b]pyridine-5-carboxamido)methyl)-4-isopropylbenzoic acid

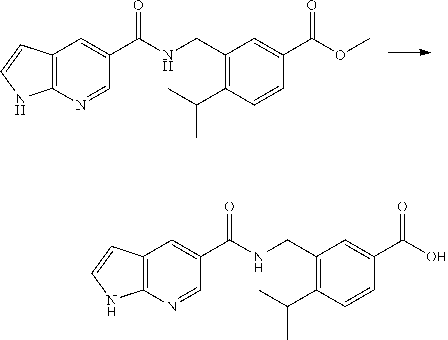 [SMILES: COC(=O)c1ccc(C(C)C)c(CNC(=O)c2cnc3[nH]ccc3c2)c1 ; CC(C)c1ccc(C(=O)O)cc1CNC(=O)c1cnc2[nH]ccc2c1]

Methyl 3-((1H-pyrrolo[2,3-b]pyridine-5-carboxamido)methyl)-4-isopropylbenzoate (0.105 g, 0.299 mmol) was dissolved in THF (3 ml) and 1 M solution of LiOH (0.65 ml, 0.448 mmol) was added to the solution and stirred over 60 h at 35° C. The reaction was concentrated in vacuo and the resulting residue was dissolved in water and acidified with 10% KHSO₄ solution until pH=4. The product was extracted with EtOAc, the combined organic layers were dried with Na₂SO₄, concentrated in vacuo to afford the titular compound (0.043 g, 43%) which was used in the further step without additional purification.

1H NMR (300 MHz, Methanol-d4) δ 8.76 (d, J=2.1 Hz, 1H), 8.52 (d, J=2.1 Hz, 1H), 8.06 (d, J=1.9 Hz, 1H), 7.95 (dd, J=8.1, 1.9 Hz, 1H), 7.51-7.46 (m, 2H), 6.62 (d, J=3.5 Hz, 1H), 4.76 (s, 2H), 3.41 (p, J=7.0 Hz, 1H), 1.32 (d, J=6.8 Hz, 6H).

Step 4: Preparation of N-(2-isopropyl-5-((3-((4-methylpiperazin-1-yl)methyl)-5-(trifluoromethyl)phenyl)carbamoyl)benzyl)-1H-pyrrolo[2,3-b]pyridine-5-carboxamide (Example 27)

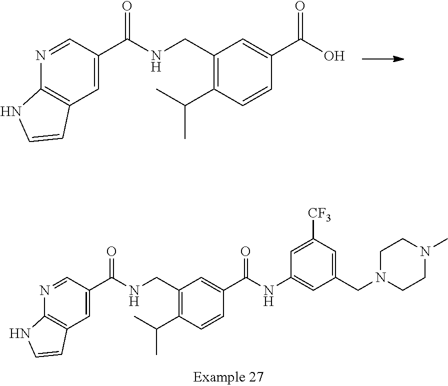 [SMILES: CC(C)c1ccc(C(=O)O)cc1CNC(=O)c1cnc2[nH]ccc2c1 ; CC(C)c1ccc(C(=O)Nc2cc(CN3CCN(C)CC3)cc(C(F)(F)F)c2)cc1CNC(=O)c1cnc2[nH]ccc2c1]

To the 3-((1H-pyrrolo[2,3-b]pyridine-5-carboxamido)methyl)-4-isopropylbenzoic acid (0.043 g, 0.127 mmol) solution in DMF (0.1 mL), BTFFH (0.121 g, 0.382 mmol) and DIPEA (0.100 ml, 0.574 mmol) were added. The mixture was stirred 15 minutes then 3-((4-methylpiperazin-1-yl)methyl)-5-(trifluoromethyl)aniline (0.052 g, 0.191 mmol) was added and the reaction mixture was stirred at 80° C. for 18 h. Then, the mixture was diluted with EtOAc, washed with water. The organic layers were then washed with brine and concentrated in vacuo. The crude material was purified via prepHPLC (ACN+0.1% FA, H₂O+0.1% FA), followed by preparative TLC (DCM:MeOH, 90:10) to afford the formic salt of the titular compound. The material obtained was dissolved in MeOH, stirred with Amberlite IRN-78 for 2 h, filtered and concentrated in vacuo to yield the title compound (0.003 g, 4% yield) as a white solid.

| | t_{R} | | |
|---|---|---|---|
| Ex. | (min) | Method | Analytical data NMR |
| Ex. 27 | 2.02 | 1 | 1H NMR (300 MHz, Methanol-d4) δ 8.75 (d, |
| | | | J = 2.1 Hz, 1H), 8.51 (d, J = 2.1 Hz, 1H), 8.06 |
| | | | (s, 1H), 7.98 (d, J = 2.0 Hz, 1H), 7.91-7.85 |
| | | | (m, 2H), 7.52 (d, J = 8.2 Hz, 1H), 7.48 (d, J = |
| | | | 3.6 Hz, 1H), 7.41 (s, 1H), 6.60 (d, J = 3.5 Hz, |
| | | | 1H), 4.77 (s, 2H), 3.60 (s, 2H), 3.43 (p, J = 6.8 |
| | | | Hz, 1H), 2.52 (s, 8H), 2.27 (s, 3H), 1.32 (d, J = |
| | | | 6.8 Hz, 6H). |

### Example 28

N-(5-((3-((4-methylpiperazin-1-yl)methyl)-5-(trifluoromethyl)phenyl)carbamoyl)-2-propylbenzyl)imidazo[1,2-a]pyridine-3-carboxamide formate salt

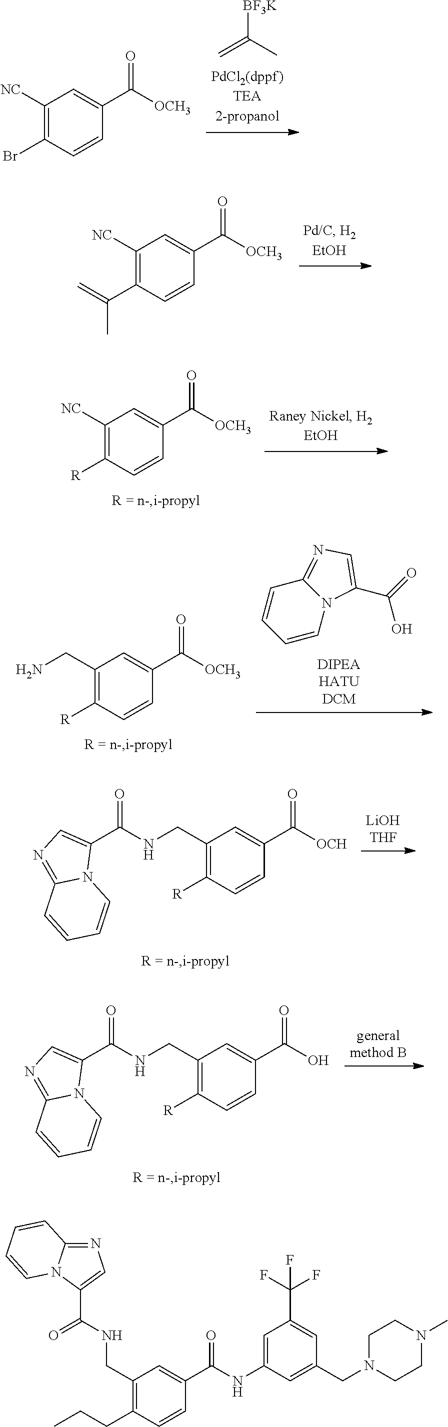

Step 1: Preparation of methyl 3-cyano-4-(prop-1-en-2-yl)benzoate; methyl 3-cyano-4-[(1E)-prop-1-en-1-yl]benzoate

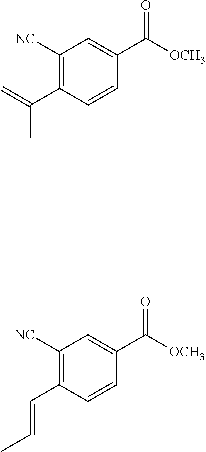

To the solution of methyl 4-bromo-3-cyanobenzoate (2.00 g, 8.33 mmol) and potassium isopropenyltrifluoroborate (2.47 g, 16.7 mmol) in iPrOH (21 mL), TEA (4.7 mL, 33.3 mmol) was added, followed by PdCl₂(dppf) (0.305 g, 0.417 mmol). The mixture was degassed with argon and stirred at 110° C. overnight. Next, the reaction mixture was concentrated to dryness and partitioned between EtOAc and water. The aqueous phase was extracted with EtOAc (3×20 mL) and washed with water (3×20 mL) and brine (20 mL). The combined organic layers were dried with Na₂SO₄, concentrated in vacuo to afford the crude, which was purified via FCC (hexane:EtOAc, from 99:1 to 90:10) to yield the product as an isomeric mixture (ratio 1:1, 1.68 g, 56%)

¹H NMR (d-DMSO, 300 MHz): δiPr 8.23 (m, 2H), 7.96 (d, J=8.4 Hz, 1H), 5.52 (t, J=1.5 Hz, 1H), 5.33 (s, 1H, CH), 3.90 (s, 3H), 2.17 (s, 3H) δnPr 8.23 (m, 2H), 7.70 (m, 1H), 6.76 (m, 2H), 3.90 (s, 3H), 1.98 (dd, J=6.2, 1.1 Hz, 3H).

Step 2: Preparation of methyl 3-cyano-4-(propan-2-yl)benzoate; methyl 3-cyano-4-propylbenzoate

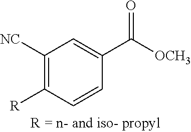

A mixture of methyl 3-cyano-4-(prop-1-en-2-yl)benzoate and methyl 3-cyano-4-[(1E)-prop-1-en-1-yl]benzoate (500 mg, 2.49 mmol, ratio 1:1) was dissolved in ethanol (100 mL) in a Parr apparatus. 10% Palladium on carbon (5.89 mg, 0.05 mmol) was added to the reaction mixture and stirred under a hydrogen atmosphere (7 atm) over 16 h. The reaction mixture was filtered through Celite, concentrated in vacuo and the mixture of methyl 3-cyano-4-(propan-2-yl)benzoate and methyl 3-cyano-4-propylbenzoate (ratio 1:1, 506 mg, 100%) was taken onto the next step without further purification.

¹H NMR (400 MHz, DMSO-d6) δiPr 8.26 (dd, J=4.8, 1.8 Hz, 2H), 7.72 (d, J=8.3 Hz, 1H), 3.88 (s, 3H), 3.29 (m, 1H), 1.29 (d, J=6.9 Hz, 6H). δnPr 8.18 (ddd, J=13.0, 8.2, 1.9 Hz, 2H), 7.64 (d, J=8.1 Hz, 1H), 3.88 (s, 3H), 2.84 (dd, J=8.4, 6.8 Hz, 2H), 1.67 (m, 2H), 0.93 (t, J=7.4 Hz, 3H).

Step 3: Preparation of methyl 3-(aminomethyl)-4-(propan-2-yl)benzoate; methyl 3-(aminomethyl)-4-propylbenzoate

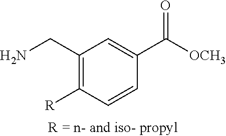

A mixture of methyl 3-cyano-4-(propan-2-yl)benzoate and methyl 3-cyano-4-propylbenzoate (ratio 1:1, 505 mg, 2.485 mmol) was dissolved in ethanol (300 mL) in a Parr apparatus. Raney Nickel (5 mL) was added to the reaction mixture and stirred under a hydrogen atmosphere (7 atm) over 16 h. The reaction mixture was filtered through Celite, concentrated in vacuo to afford the crude material, which was purified by column chromatography (DCM: 5.5 M NH₃ in MeOH, from 99:1 to 98:2), to yield the mixture of methyl 3-(aminomethyl)-4-(propan-2-yl)benzoate and methyl 3-(aminomethyl)-4-propylbenzoate (ratio: 1:1, 118 mg, 23%)

¹H NMR (d-DMSO, 400 MHz): iPr: δ 8.02 (d, J=1.9 Hz, 1H), 7.79 (dd, J=8.1, 2.0 Hz, 1H), 7.41 (d, J=8.1 Hz, 1H), 3.84 (s, 3H), 3.79 (s, 2H), 3.26 (sept, J=6.9 Hz, 1H), 1.84 (m, 2H), 1.20 (d, J=6.8 Hz, 6H). nPr: δ 8.06 (d, J=2.0 Hz, 1H), 7.74 (dd, J=7.9, 2.0 Hz, 1H), 7.27 (d, J=7.9 Hz, 1H), 3.84 (s, 3H), 3.83 (s, 2H) 2.64 (m, 2H), 1.57 (m, 2H), 0.94 (t, J=7.3 Hz)

Step 4: Preparation of methyl 3-[({imidazo[1,2-a]pyridin-3-yl}formamido)methyl]-4-(propan-2-yl)benzoate; methyl 3-[({imidazo[1,2-a]pyridin-3-yl}formamido)methyl]-4-propylbenzoate

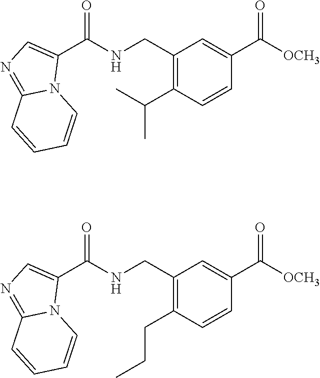 [SMILES: COC(=O)c1ccc(C(C)C)c(CNC(=O)c2cnc3ccccn23)c1 ; CCCc1ccc(C(=O)OC)cc1CNC(=O)c1cnc2ccccn12]

To the solution of methyl 3-(aminomethyl)-4-isopropylbenzoate and methyl 3-(aminomethyl)-4-propylbenzoate (ratio 1:1, 0.156 g, 0.748 mmol), imidazo[1,2-a]pyridine-3-carboxylic acid (0.146 g, 0.897 mmol) and HATU (0.284 g, 0.748 mmol) in DCM (7.5 mL), DIPEA was added (0.26 mL, 1.50 mmol) and the mixture was stirred at RT for 16 h. The reaction was quenched by addition of water, then it was extracted with DCM (3×25 mL). The combined organic layers were washed with water (25 mL), brine (20 mL), dried with Na₂SO₄, filtered and concentrated in vacuo. The crude material was purified via column chromatography (DCM:MeOH, 98:2), followed by prepHPLC to yield isopropyl (93 mg, 71%) and n-propyl (108 mg, 82%) isomers.

¹H NMR (300 MHz, d-DMSO): δ (methyl 3-((imidazo[1,2-a]pyridine-3-carboxamido)methyl)-4-isopropylbenzoate) 9.48 (d, J=6.9 Hz, 1H), 9.03 (t, J=5.7 Hz, 1H), 8.41 (s, 1H), 7.95 (d, J=1.9 Hz, 1H), 7.86 (dd, J=8.1, 1.9 Hz, 1H), 7.73 (dt, J=9.0, 1.2 Hz, 1H), 7.54-7.43 (m, 2H), 7.13 (td, J=6.9, 1.3 Hz, 1H), 4.62 (d, J=5.6 Hz, 2H), 3.82 (s, 3H), 3.40 (d, J=6.7 Hz, 1H), 1.23 (d, J=6.8 Hz, 6H).

δ (methyl 3-((imidazo[1,2-a]pyridine-3-carboxamido)methyl)-4-propylbenzoate) 9.48 (dt, J=7.0, 1.2 Hz, 1H, CH), 9.05 (t, J=5.8 Hz, 1H, NH), 8.42 (s, 1H, CH), 7.95 (d, J=1.9 Hz, 1H, CH), 7.80 (dd, J=7.9, 1.9 Hz, 1H, CH), 7.73 (dt, J=9.1, 1.2 Hz, 1H, CH), 7.47 (ddd, J=9.1, 6.8, 1.4 Hz, 1H, CH), 7.36 (d, J=8.0 Hz, 1H, CH), 7.13 (td, J=6.9, 1.3 Hz, 1H, CH), 4.59 (d, J=5.7 Hz, 2H, CH₂), 3.81 (s, 3H, OCH₃), 2.79-2.70 (m, 2H, CH₂), 1.68-1.53 (m, 2H), 0.94 (t, J=7.3 Hz, 3H).

Step 5: Preparation of lithium 3-((imidazo[1,2-a]pyridine-3-carboxamido)methyl)-4-propylbenzoate

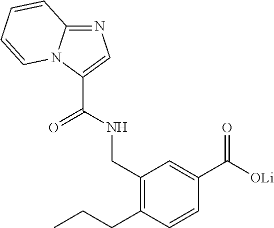

Methyl 3-((imidazo[1,2-a]pyridine-3-carboxamido)methyl)-4-isopropylbenzoate (108 mg, 0.293 mmol) was dissolved in THF (3 mL) and a 1 M solution of LiOH in H₂O (0.70 mL, 0.700 mmol) was added and the reaction mixture was stirred at RT over 3 d. The reaction mixture was concentrated in vacuo and triturated with diethyl ether to yield the titular product as an off-white solid (114 mg, 100%).

1H NMR (300 MHz, DMSO-d6) δ 9.54 (d, J=7.0 Hz, 1H), 8.96 (s, 1H), 8.42 (s, 1H), 7.85 (d, J=1.5 Hz, 1H), 7.71 (d, J=9.0 Hz, 1H), 7.66 (dd, J=7.8, 1.6 Hz, 1H), 7.49-7.41 (m, 1H), 7.11 (t, J=7.0 Hz, 1H), 7.05 (d, J=7.7 Hz, 1H), 4.53 (s, 2H), 2.68-2.59 (m, 2H), 1.64-1.50 (m, 2H), 0.92 (t, J=7.3 Hz, 3H).

Step 6: Preparation of N-(5-((3-((4-methylpiperazin-1-yl)methyl)-5-(trifluoromethyl)phenyl)carbamoyl)-2-propylbenzyl)imidazo[1,2-a]pyridine-3-carboxamide formate salt (Example 28)

Preparations of Example 28 was performed according to the General method B for amide coupling, reacting lithium 3-((imidazo[1,2-a]pyridine carboxamido)methyl)-4-propylbenzoate with the required amines to give the following compounds:

| | | | Amount | | | |
|---|---|---|---|---|---|---|
| | Structure and | Amount | product | LCMS | t_{R} | |
| Ex. | chemical name | SM | (yield) | Method | (min) | NMR data |
| Ex. 28 | 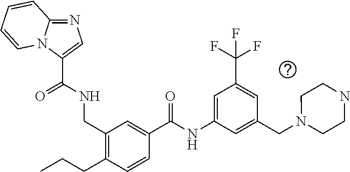 [SMILES: CCCc1ccc(C(=O)Nc2cc(CN3CCNCC3)cc(C(F)(F)F)c2)cc1CNC(=O)c1cnc2ccccn12] | 0.059 g | 0.010 g (10%) | 1 | 1.817 | 1H NMR (300 MHz, DMSO-d6) δ 10.48 (s, 1H), 9.49 (d, J = 7.0 Hz, 1H), 9.02 (d, J = 5.7 Hz, 1H), 8.42 (s, 1H), 8.14 (s, 2H), 8.04- 7.92 (m, 2H), 7.90- 7.83 (m, 1H), 7.72 (d, J = 8.9 Hz, 1H), 7.47 (dd, J = |
| | | | | | | 8.7, 6.9 Hz, 1H), |
| | | | | | | 7.42-7.26 (m, |
| | | | | | | 2H), 7.13 (t, J = 6.7 |
| | | | | | | Hz, 1H), 4.62 (d, |
| | | | | | | J = 5.6 Hz, 2H), 3.54 |
| | | | | | | (s, 2H), 1.63 (q, J = |
| | | | | | | 7.5 Hz, 2H), 0.96 |
| | | | | | | (t, J = 7.3 Hz, 3H). |

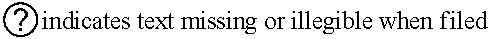

### Example 29

N-(3-((4-methylpiperazin-1-yl)methyl)-5-(trifluoromethyl)phenyl)-4-propyl-3-((pyrimidin-5-ylamino)methyl)benzamide

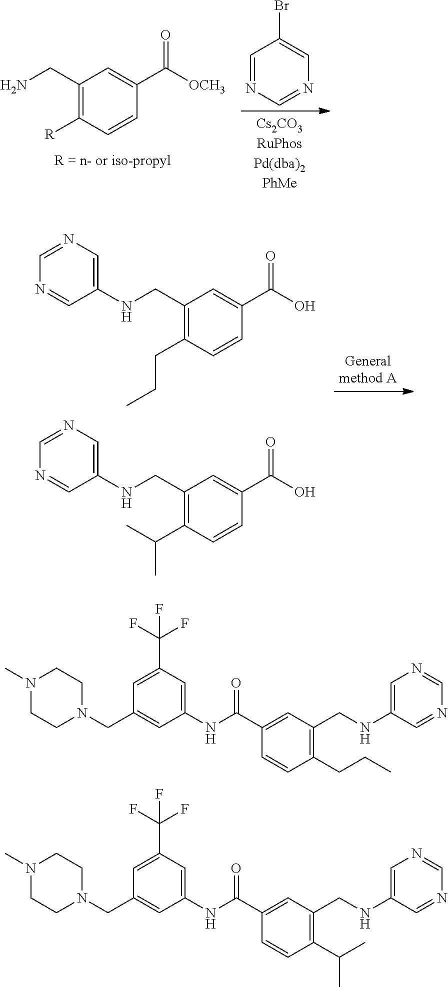

Step 1: Preparation of 4-propyl-3-((pyrimidin-5-ylamino)methyl)benzoic acid and 4-isopropyl-3-((pyrimidin-5-ylamino)methyl)benzoic acid

To a mixture of methyl 3-(aminomethyl)-4-isopropylbenzoate and methyl 3-(aminomethyl)-4-propylbenzoate (ratio 1:1, 200 mg, 0.964 mmol), prepared as in Example 16, step 1-2, 5-bromopyrimidine (184 mg, 1.158 mmol) and Cs₂(CO)₃ (943 mg, 2.89 mmol) were added, followed by toluene (2 mL). The mixture was degassed and RuPhos (90 mg, 0.193 mmol) and Pd(dba)₂ (55.5 mg, 0.096 mmol) were added and the reaction mixture was stirred at 110° C. overnight. The reaction mixture was filtered through Celite and concentrated in vacuo to give the crude, which was purified by column chromatography (DCM:MeOH, from 99:1 to 0:100), followed by prepHPLC to obtain the iso- (69 mg, 53%) and n-propyl (87 mg, 67%) isomers.

¹H NMR iPr (300 MHz, DMSO-d6) δ 8.40 (s, 1H), 8.14 (s, 2H), 7.86 (d, J=1.7 Hz, 1H), 7.84-7.77 (m, 1H), 7.38 (d, J=8.0 Hz, 1H), 6.56 (t, J=5.5 Hz, 1H), 4.36 (d, J=5.2 Hz, 2H), 3.23 (sept., J=6.8 Hz, 1H), 1.22 (d, J=6.8 Hz, 6H). ¹H NMR nPr (300 MHz, DMSO-d6) δ 9.32 (s, 1H), 8.38 (s, 1H), 8.12 (s, 2H), 7.82 (d, J=1.7 Hz, 1H), 7.69 (dd, J=7.8, 1.7 Hz, 1H), 7.12 (d, J=7.8 Hz, 1H), 6.58 (t, J=5.5 Hz, 1H), 4.29 (d, J=5.4 Hz, 2H), 2.63 (dd, J=8.9, 6.5 Hz, 2H), 1.68-1.49 (m, 2H), 0.93 (t, J=7.3 Hz, 3H).

Step 2: Preparation of N-(3-((4-methylpiperazin-1-yl)methyl) (trifluoromethyl)phenyl)-4-propyl-3-((pyrimidin-5-ylamino)methyl)benzamide (Example 29) and 4-isopropyl-N-(3-((4-methylpiperazin-1-yl)methyl) (trifluoromethyl)phenyl)-3-((pyrimidin-5-ylamino)methyl)benzamide (Example 30).

Preparations of Example 29 and Example 30 was performed according to the General method B for amide coupling, reacting 4-propyl-3-((pyrimidin-5-ylamino)methyl)benzoic acid and 4-isopropyl-3-((pyrimidin-5-ylamino)methyl)benzoic acid with the required amines to give the following compounds:

| | | | Amount | | | |
|---|---|---|---|---|---|---|
| | Structure and | Amount | product | | t_{R} | Analytical data NMR |
| Ex. | chemical name | SM | (yield) | Method | (min) | data |
| Ex.29 | 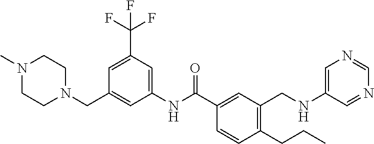 [SMILES: CCCc1ccc(C(=O)Nc2cc(CN3CCN(C)CC3)cc(C(F)(F)F)c2)cc1CNc1cncnc1] | 0.044 g | 0.012 g (14%) | 1 | 1.970 | 1H NMR (300 MHz, Methanol-d4) δ 8.41 (s, 1H), 8.16 (s, 2H), 8.04 (s, 1H), 7.97 (d, J = 1.9 Hz, 1H), 7.89 (s, 1H), 7.85 (dd, J = 8.0, 2.0 Hz, 1H), 7.45- 7.38 (m, 2H), 4.46 (s, 2H), 3.61 (s, 2H), 2.84- |
| | N-(3-((4-methylpiperazin- | | | | | 2.75 (m, 2H), 2.53 |
| | 1-yl)methyl)-5- | | | | | (s, 8H), 2.29 (s, 3H), |
| | (trifluoromethyl)phenyl)-4- | | | | | 1.78-1.67 (m, 2H), |
| | propyl-3-((pyrimidin-5- | | | | | 1.02 (t, J = 7.3 Hz, |
| | ylamino)methyl)benzamide | | | | | 3H). |
| Ex.30 | 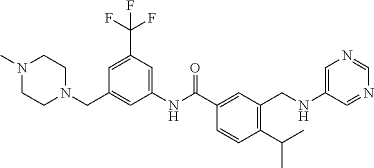 [SMILES: CC(C)c1ccc(C(=O)Nc2cc(CN3CCN(C)CC3)cc(C(F)(F)F)c2)cc1CNc1cncnc1] | 0.044 g | 0.017 g (19%) | 1 | 1.930 | 1H NMR (300 MHz, Methanol-d4) δ 8.41 (s, 1H), 8.18 (s, 2H), 8.03 (s, 1H), 7.97 (d, J = 2.0 Hz, 1H), 7.91 (dd, J = 10.7, 2.5 Hz, 2H), 7.55 (d, J = 8.2 Hz, 1H), 7.42 (s, 1H), 4.48 (s, 2H), 3.64 (s, 2H), 2.67 (d, J = 31.8 Hz, 8H), 2.44 (s, 3H), |
| | 4-isopropyl-N-(3-((4- | | | | | 1.32 (d, J = 6.8 Hz, |
| | methylpiperazin-1- | | | | | 6H). |
| | yl)methyl)-5- | | | | | |
| | (trifluoromethyl)phenyl)-3- | | | | | |
| | ((pyrimidin-5- | | | | | |
| | ylamino)methyl)benzamide | | | | | |

### Example 33

34(1H-pyrrolo[2,3-b]pyridin-5-yl)amino)methyl)-4-fluoro-N-(3-(trifluoromethyl)phenyl)benzamide

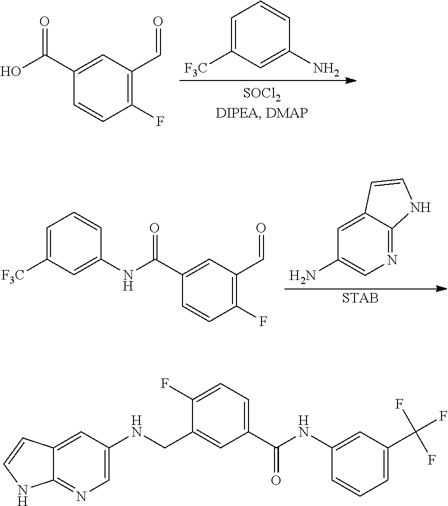

Step 1: Preparation of 4-fluoro-3-formyl-N-(3-(trifluoromethyl)phenyl)benzamide A solution of 4-fluoro-3-formylbenzoic acid (200 mg, 1.190 mmol) in SOCl₂ (2.386 ml, 32.7 mmol) was refluxed for 2 h, then evaporated in vacuo to remove residual SOCl₂. The brown, solid residue was dissolved in anhydrous THF (4.0 ml) and added dropwise to a solution of DIPEA (0.249 ml, 1.428 mmol), 3-(trifluoromethyl)aniline (192 mg, 1.190 mmol) and DMAP (5.81 mg, 0.048 mmol) in anhydrous THF (2.0 ml). The reaction mixture was stirred at RT for 18 h. Reaction mixture was quenched by addition to water (20 mL). 1 M NaOH (4 mL) was added to adjust pH to 10. Product was extracted with AcOEt (3×25 mL), combined organic extracts were evaporated in vacuo to give crude product (548 mg). Crude was purified by column chromatography (Hexane/DCM, from 1:3 to 0:1) to give the title product (265 mg, 72%).

¹H NMR (300 MHz, DMSO-d6) δ 10.76 (s, 1H), 10.30 (s, 1H), 8.51 (dd, J=6.7, 2.5 Hz, 1H), 8.35 (ddd, J=8.7, 5.0, 2.5 Hz, 1H), 8.23 (d, J=2.2 Hz, 1H), 8.07 (d, J=8.3 Hz, 1H), 7.67-7.58 (m, 2H), 7.52-7.46 (m, 1H).

Step 2: Preparation of 3-(((1H-pyrrolo[2,3-b]pyridin-5-yl)amino)methyl)-4-fluoro-N-(3-(trifluoromethyl)phenyl)benzamide

4-fluoro-3-formyl-N-(3-(trifluoromethyl)phenyl)benzamide (80 mg, 0.257 mmol) and 1H-pyrrolo[2,3-b]pyridin-5-amine (34.2 mg, 0.257 mmol) were placed in round bottom flask under argon. Glacial AcOH (1.0 ml) was added and reaction mixture was stirred 3 h at RT. Reaction mixture was cooled down in ice water then STAB (163 mg, 0.771 mmol) as suspension in glacial Acetic Acid (1.0 ml) was added and reaction mixture stirred at RT for 72 h. Reaction mixture was added to 1M NaOH (50 mL) and aqueous layer was extracted with AcOEt (3×25 mL), organic layers were combined, dried (Na₂SO₄), filtered and evaporated to give crude product (126 mg). Crude product was purified by preparative TLC (SiO₂, DCM/MeOH 100:5) to give the title compound (35.26 mg. 32%).

| | t_{R} | | |
|---|---|---|---|
| Ex. | (min) | Method | Analytical data NMR |
| Ex. 33 | 3.77 | 6 | ¹H NMR (300 MHz, DMSO-d6) δ 11.14 (s, |
| | | | 1H), 10.53 (s, 1H), 8.18 (s, 1H), 8.10 (dd, J = |
| | | | 7.3, 2.4 Hz, 1H), 8.00 (d, J = 9.1 Hz, 1H), |
| | | | 7.98-7.91 (m, 1H), 7.83 (d, J = 2.6 Hz, 1H), |
| | | | 7.58 (t, J = 8.0 Hz, 1H), 7.47-7.35 (m, 2H), |
| | | | 7.24 (t, J = 2.9 Hz, 1H), 7.04 (d, J = 2.6 Hz, |
| | | | 1H), 6.18 (dd, J = 3.3, 1.8 Hz, 1H), 5.94 |
| | | | (t, J = 6.2Hz, 1H), 4.39 (d, J = 6.1 Hz, 2H). |

### Example 34

4-methyl-3-((pyridin-3-ylamino)methyl)-N-(3-(trifluoromethyl) phenyl)benzamide

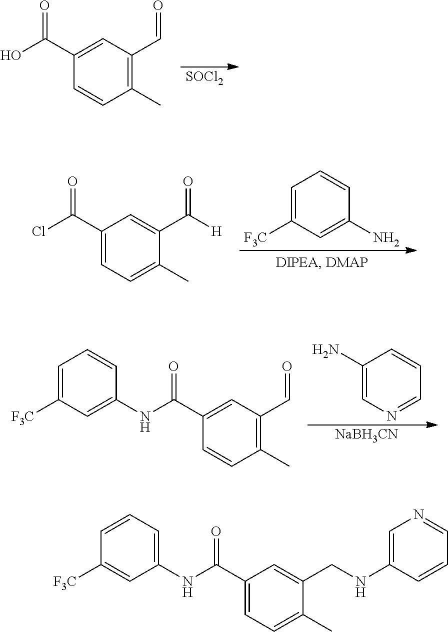

Step 1: Preparation of 3-formyl-4-methylbenzoyl chloride

3-formyl-4-methylbenzoic acid (1 g, 6.09 mmol) was dissolved in DCM (20.31 ml). The solution was cooled down to 0° C., then Oxalyl chloride (1.569 ml, 18.27 mmol) and DMF (catalytic amount) were added. The reaction mixture was stirred in ice bath for 3 h. Formation of acid chloride was confirmed by quenching of reaction with MeOH (methyl ester). The mixture was concentrated to give the desired product (1.1 g, 99%) and that material was used into the next step without further purification.

Step 2: Preparation of 3-formyl-4-methyl-N-(3-(trifluoromethyl)phenyl)benzamide

3-formyl-4-methylbenzoyl chloride (1 g, 5.48 mmol) was dissolved in THF (5.37 ml) and this solution was added to solution of 3-(trifluoromethyl)aniline (0.684 ml, 5.48 mmol), DIPEA (1.145 ml, 6.57 mmol) and DMAP (0.027 g, 0.219 mmol) in THF (10.74 ml). The mixture was stirred at RT overnight. The reaction mixture was concentrated. The crude material was dissolved in sat. NaHCO₃ and extracted with DCM (×3). The all combined organic layers were washed with 5% citric acid, dried over Na₂SO₄, filtered and concentrated. The crude material was purified via FCC (from 100% Hexane to 30% AcOEt in Hexane) to give desired product (1.13 g, 67%).

¹H NMR (300 MHz, DMSO-d6) δ 10.68 (s, 1H), 10.33 (s, 1H), 8.46 (d, J=2.1 Hz, 1H), 8.25 (d, J=2.0 Hz, 1H), 8.16 (dd, J=8.0, 2.1 Hz, 1H), 8.07 (dt, J=7.9, 2.3 Hz, 1H), 7.62 (t, J=8.0 Hz, 1H), 7.54 (d, J=8.0 Hz, 1H), 7.51-7.44 (m, 1H), 2.71 (s, 3H).

Step 3: Preparation of 4-methyl-3-((pyridin-3-ylamino)methyl)-N-(3-(trifluoromethyl) phenyl)benzamide

The 3-formyl-4-methyl-N-(3-(trifluoromethyl)phenyl)benzamide (0.1 g, 0.325 mmol) and pyridin-3-amine (0.031 g, 0.325 mmol) were dissolved in MeOH (1.63 ml) and AcOH (0.06 ml). The mixture was stirred at 50° C. for 1 h, then reaction mixture was cooled down toRT and NaBH₃CN (0.092 g, 1.464 mmol) was added. The solution was stirred at 50° C. for 1 h. The reaction mixture was cooled down to RT and quenched with 1M NaOH aq. solution, product was extracted with AcOEt (×3). The all combined organic layers were dried over Na₂SO₄, filtered and concentrated. The crude material was purified via FCC (from DCM 100% to 10% MeOH in DCM), then it was repurified via preparative HPLC (ACN+0.1% FA, H₂O+0.1% FA). The obtained product was washed with saturated NaHCO₃ to remove formic acid to give the desired product (48 mg, 38%).

| | t_{R} | | |
|---|---|---|---|
| Ex. | (min) | Method | Analytical data NMR |
| Ex. 34 | 1.51 | 6 | ¹H NMR (300 MHz, Methanol-d4) δ 8.12 (s, |
| | | | 1H), 7.96 (d, J = 2.1 Hz, 2H), 7.91 (d, J = 8.9 |
| | | | Hz, 1H), 7.84-7.73 (m, 2H), 7.53 (t, J = 8.0 |
| | | | Hz, 1H), 7.41 (d, J = 8.8 Hz, 1H), 7.37 (d, J = |
| | | | 8.0 Hz, 1H), 7.16 (dd, J = 8.3, 4.6 Hz, 1H), |
| | | | 7.08-7.00 (m, 1H), 4.39 (s, 2H), 2.48 (s, 3H). |

### Example 35

4-fluoro-3-(((5-(1-methyl-1H-pyrazol-3-yl)pyridin-3-yl)amino)methyl)-N-(3-(trifluoromethoxy)phenyl)benzamide

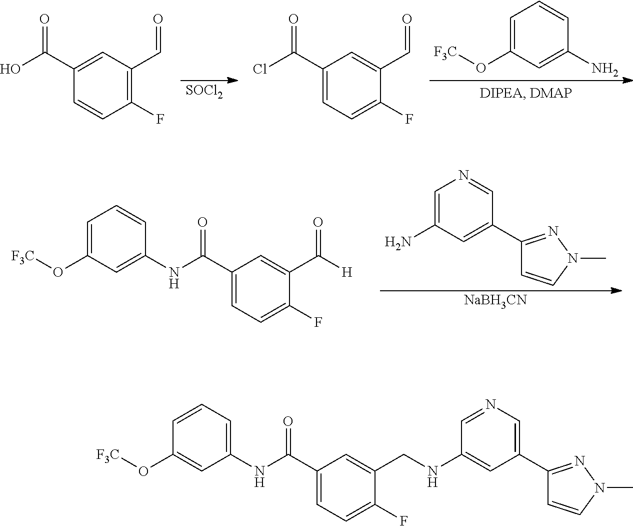

Step 1: Preparation of 4-fluoro-3-formylbenzoyl chloride

4-fluoro-3-formylbenzoic acid (0.2 g, 1.190 mmol) was dissolved in DCM (5.95 ml). The solution was cooled down to 0° C., then oxalyl chloride (0.306 ml, 3.57 mmol) and DMF (catalytic amount) were added. The mixture was stirred in ice bath for 3 h. Formation of acid chloride was confirmed by quenching of reaction with MeOH (methyl ester). The reaction mixture was concentrated (222 mg, 100%) and that material was used into the next step without other purification.

Step 2: Preparation of 4-fluoro-3-formyl-N-(3-(trifluoromethoxy) phenyl)benzamide

The 4-fluoro-3-formylbenzoyl chloride (0.2 g, 1.072 mmol) was dissolved in THF (1.083 ml) and this solution was added to solution of 3-(trifluoromethoxy)aniline (0.172 ml, 1.286 mmol), DIPEA (0.224 ml, 1.286 mmol) and DMAP (5.24 mg, 0.043 mmol) in THF (2.166 ml). The mixture was stirred atRT overnight. The reaction mixture was diluted with sat. NaHCO₃ and extracted with AcOEt (×3). The all combined organic layers were washed with 5% citric acid, dried over Na₂SO₄, filtered and concentrated. The crude material was purified via FCC (from 100% Hexane to 50% AcOEt in Hexane) to give desired product (227 mg, 65%).

¹H NMR (300 MHz, DMSO-d6) δ 10.71 (s, 1H), 10.29 (s, 1H), 8.48 (dd, J=6.7, 2.4 Hz, 1H), 8.33 (ddd, J=8.7, 5.0, 2.5 Hz, 1H), 7.92 (dq, J=2.3, 1.1 Hz, 1H), 7.78 (ddd, J=8.3, 2.0, 0.9 Hz, 1H), 7.61 (dd, J=10.3, 8.7 Hz, 1H), 7.51 (t, J=8.2 Hz, 1H), 7.12 (ddt, J=8.2, 2.4, 1.1 Hz, 1H).

Step 3: Preparation of 4-fluoro-3-(((5-(1-methyl-1H-pyrazol-3-yl)pyridin-3-yl)amino)methyl)-N-(3-(trifluoromethoxy)phenyl)benzamide

The 4-fluoro-3-formyl-N-(3-(trifluoromethoxy)phenyl)benzamide (0.1 g, 0.306 mmol) and 5-(1-methyl-1H-pyrazol-3-yl)pyridin-3-amine (0.053 g, 0.306 mmol) were dissolved in MeOH (1.528 ml) and AcOH (0.053 ml, 0.917 mmol). The mixture was stirred at 50° C. for 1 h, then it was cooled down to RT then NaBH₃CN (0.086 g, 1.375 mmol) was added. The solution was stirred at 50° C. for 1 h. The reaction mixture was cooled down to RT and quenched with sat. NaHCO₃, product was extracted with AcOEt (×3). The all combined organic layers were dried over Na₂SO₄, filtered and concentrated. The crude material was purified via FCC (from 100% DCM to 10% MeOH in DCM), then it was repurified via preparative HPLC (ACN+0.1% NH₃, H₂O+0.1% NH₃) to give the desired product (47 mg, 32%).

| | t_{R} | | |
|---|---|---|---|
| Ex. | (min) | Method | Analytical data NMR |
| Ex. 35 | 2.18 | 1 | ¹H NMR (400 MHz, Methanol-d4) δ 8.10 (dd, |
| | | | J = 7.1, 2.4 Hz, 1H), 8.00 (d, J = 1.8 Hz, 1H), |
| | | | 7.99 (s, 1H), 7.91 (ddd, J = 8.3, 4.8, 2.4 Hz, |
| | | | 1H), 7.82 (d, J = 2.7 Hz, 1H), 7.80 (d, J = 0.9 |
| | | | Hz, 2H), 7.62 (ddd, J = 8.2, 2.0, 0.9 Hz, 1H), |
| | | | 7.42 (t, J = 8.2 Hz, 1H), 7.28 (dd, J = 9.7, 8.5 |
| | | | Hz, 1H), 7.20 (dd, J = 2.7, 1.8 Hz, 1H), 7.07- |
| | | | 7.01 (m, 1H), 4.53 (s, 2H), 3.92 (s, 3H). |

The following compound was prepared via reductive amination as described for Example 35, step 1-3, applying the corresponding commercially available amine in step 3 and using STAB as reductive agent.

| | | | Amount | | | |
|---|---|---|---|---|---|---|
| | Structure and | Amount | product | LCMS | t_{R} | |
| Ex. | chemical name | SM | (yield) | Method | (min) | NMR data |
| Ex. 38 | 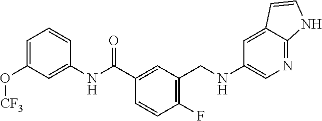 [SMILES: O=C(Nc1cccc(OC(F)(F)F)c1)c1ccc(F)c(CNc2cnc3[nH]ccc3c2)c1] | 0.050 g | 0.032 g (47%) | 6 | 2.37 | ¹H NMR (300 MHz, DMSO-d6) δ 11.14 (s, 1H), 10.48 (s, 1H), 8.09 (dd, J = 7.3, 2.3 Hz, 1H), 7.94 (ddd, J = 7.8, 4.9, 2.4 Hz, 1H), 7.88 (s, 1H), |
| | 3-(((1H-pyrrolo[2,3-b]pyridin- | | | | | 7.83 (d, J = 2.6 Hz, |
| | 5-yl)amino)methyl)-4-fluoro- | | | | | 1H), 7.74-7.69 |
| | N-(3- | | | | | (m, 1H), 7.46 (t, J = |
| | (trifluoromethoxy)phenyl) | | | | | 8.2 Hz, 1H), 7.39 |
| | benzamide | | | | | (dd, J = 9.9, 8.6 Hz, |
| | | | | | | 1H), 7.24 (t, J = 2.9 |
| | | | | | | Hz, 1H), 7.07 (d, |
| | | | | | | J = 7.2 Hz, 1H), 7.04 |
| | | | | | | (d, J = 2.7 Hz, 1H), |
| | | | | | | 6.18 (dd, J = 3.4, |
| | | | | | | 1.8 Hz, 1H), 5.94 |
| | | | | | | (t, J = 6.2 Hz, 1H), |
| | | | | | | 4.39 (d, J = 6.1 Hz, |
| | | | | | | 2H). |

### Example 36

4-(difluoromethyl)-N-(3-(4-methyl-1H-imidazol-1-yl)-5-(trifluoromethyl) phenyl)-3-((pyrimidin-5-ylamino)methyl)benzamide

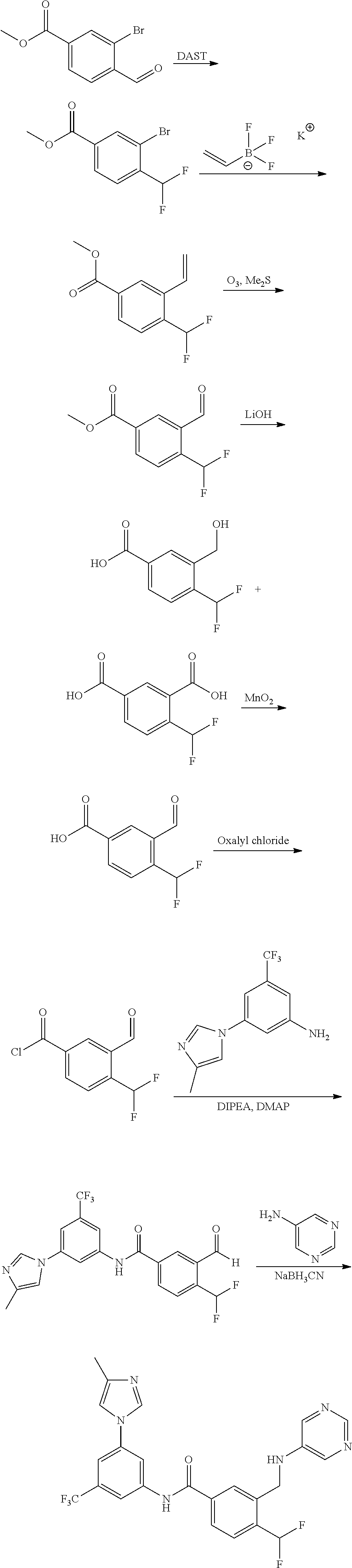

Step 1: Preparation of methyl 3-bromo-4-(difluoromethyl)benzoate Methyl 3-bromo-4-formylbenzoate (5 g, 20.57 mmol) was dissolved in anhydrous DCM (103 ml) and the solution was cooled down to 0° C. Next DAST (4.08 ml, 30.9 mmol) was added and the reaction mixture was stirred at RT overnight. The mixture was quenched with saturated NaHCO₃ and extraction was done with DCM (×3). All organic layers were dried over Na₂SO₄, filtered and concentrated under vacuum to give the desired product, which was used into the next step without further purification (5.37 g, 98%).

¹H NMR (300 MHz, Chloroform-d) δ 8.28 (q, J=1.4 Hz, 1H), 8.07 (dt, J=8.1, 1.0 Hz, 1H), 7.73 (d, J=8.1 Hz, 1H), 6.92 (t, J=54.5 Hz, 1H), 3.95 (s, 3H).

Step 2: Preparation of methyl 4-(difluoromethyl)-3-vinylbenzoate

In an oven dried pressure reactor methyl 3-bromo-4-(difluoromethyl)benzoate (5.37 g, 20.26 mmol), potassium trifluoro(vinyl)borate (5.43 g, 40.5 mmol), K₂CO₃ (7.00 g, 50.7 mmol) were placed and Dioxane (57.9 ml) was added via syringe under argon atmosphere. Solution was filled with argon (10 minutes), then Pd(dppf)Cl₂ (1.482 g, 2.026 mmol) was added. The tube was sealed and heated overnight at 110° C. The reaction mixture was filtered through a pad of celite and washed with AcOEt. Filtrate was concentrated and the crude material was purified via automatic FCC (eluting system: from 100% Hexane to 10% AcOEt in Hexane) to give the desired product (2.49 g, 58%).

¹H NMR (300 MHz, Chloroform-d) δ 8.23 (t, J=1.2 Hz, 1H), 8.01 (dt, J=8.1, 1.1 Hz, 1H), 7.64 (d, J=8.1 Hz, 1H), 7.07 (d, J=1.6 Hz, 1H), 6.84 (t, J=54.9 Hz, 2H), 5.82 (dd, J=17.4, 0.9 Hz, 1H), 5.52 (dd, J=11.1, 0.9 Hz, 1H), 3.95 (s, 3H).

Step 3: Preparation of methyl 4-(difluoromethyl)-3-formylbenzoate

Methyl 4-(difluoromethyl)-3-vinylbenzoate (2.37 g, 11.17 mmol) was dissolved in anhydrous DCM (55.8 ml) and the solution was cooled down to −78° C. Then the reaction was bubbled with ozone for 20 min. After that time the ozone flow was replaced with argon flow. Then Me₂S (1.230 ml, 16.75 mmol) was added and the mixture was stirred at −78° C. for 30 min followed by another 30 min at RT. The solvent was evaporated and the crude material was purified via FCC (from 100% Hexane to 30% AcOEt in Hexane) to give the desired product (1.73, 72%).

¹H NMR (300 MHz, Chloroform-d) δ 10.21 (s, 1H), 8.58 (s, 1H), 8.36 (dd, J=8.1, 1.8 Hz, 1H), 7.93 (d, J=8.0 Hz, 1H), 7.47 (t, J=54.6 Hz, 1H), 4.00 (s, 3H).

Step 4: Preparation of a mixture of 4-(difluoromethyl)-3-(hydroxymethyl)benzoic acid and 4-(difluoromethyl)isophthalic acid

Methyl 4-(difluoromethyl)-3-formylbenzoate (1.73 g, 8.08 mmol) was dissolved in MeOH (40.4 ml), then 1M LiOH (32.3 ml, 32.3 mmol) was added to the solution. The mixture was stirred for 1 h at RT. A mixture of alcohol and carboxylic acid was obtained since Cannizzaro dismutation occurred. The crude was extracted with AcOEt:1M HCl. The mixture of alcohol (0.76 g, 46%) and acid (0.76 g, 44%) was concentrated and used as such in the next step.

Step 5: Preparation of 4-(difluoromethyl)-3-formylbenzoic acid

4-(difluoromethyl)-3-(hydroxymethyl)benzoic acid (1.42 g, 7.02 mmol) was dissolved in Acetonitrile (46.8 ml), then MnO₂ (1.832 g, 21.07 mmol). The mixture was stirred at 80° C. overnight, then the reaction mixture was cooled down to RT and filtered through a Celite pad. The filtrate was concentrated and the crude material was purified via FCC (from 100% DCM to 10% MeOH in DCM) to give the desired product (197 mg, 14%).

¹H NMR (300 MHz, DMSO-d6) δ 13.65 (s, 1H), 10.24 (s, 1H), 8.58 (d, J=1.5 Hz, 1H), 8.34 (dd, J=8.0, 1.8 Hz, 1H), 7.96 (d, J=8.1 Hz, 1H), 7.65 (t, J=54.5 Hz, 1H).

Step 6: Preparation of 4-(difluoromethyl)-3-formylbenzoyl chloride

4-(difluoromethyl)-3-formylbenzoic acid (0.19 g, 0.949 mmol) was dissolved in DCM (4.75 ml). The solution was cooled down to 0° C., then Oxalyl chloride (0.245 ml, 2.85 mmol) and DMF (catalytic amount) were added. The reaction mixture was stirred in ice bath for 3 h, then it was concentrated and that material was used into the next step without further purification.

Step 7: Preparation of 4-(difluoromethyl)-3-formyl-N-(3-(4-methyl-1H-imidazol-1-yl)-5-(trifluoro methyl)phenyl)benzamide 4-(difluoromethyl)-3-formylbenzoyl chloride (0.19 g, 0.869 mmol) was dissolved in THF (0.852 ml) and this solution was added to solution of 3-(4-methyl-1H-imidazol-1-yl)-5-(trifluoromethyl)aniline (0.210 g, 0.869 mmol), DIPEA (0.182 ml, 1.043 mmol) and DMAP (4.25 mg, 0.035 mmol) in THF (1.704 ml). Reaction mixture was stirred at RT overnight. The reaction mixture was concentrated and the crude material was dissolved in 1M NaOH and extracted with AcOEt (×3). The combined organic layers were dried over Na₂SO₄, filtered and concentrated and the crude material was purified via FCC (from 100% DCM to 10% MeOH in DCM) to give the desired compound (146 mg, 40%).

¹H NMR (300 MHz, DMSO-d6) δ 11.02 (s, 1H), 10.30 (d, J=1.2 Hz, 1H), 8.66 (d, J=1.7 Hz, 1H), 8.41 (dd, J=8.1, 1.9 Hz, 1H), 8.29 (d, J=2.0 Hz, 1H), 8.23 (d, J=1.4 Hz, 1H), 8.14 (d, J=1.8 Hz, 1H), 8.04 (d, J=8.1 Hz, 1H), 7.79 (d, J=1.9 Hz, 1H), 7.51 (t, J=1.2 Hz, 1H), 2.19 (d, J=1.0 Hz, 3H).

Step 8: Preparation of 4-(difluoromethyl)-N-(3-(4-methyl-1H-imidazol-1-yl)-5-(trifluoromethyl)phenyl)-3-((pyrimidin-5-ylamino)methyl)benzamide

4-(difluoromethyl)-3-formyl-N-(3-(4-methyl-1H-imidazol-1-yl)-5-(trifluoro methyl)phenyl)benzamide (0.06 g, 0.142 mmol) and pyrimidin-5-amine (0.013 g, 0.142 mmol) were dissolved in MeOH (0.71 ml) and AcOH (0.024 ml). Molecular sieves were added and the mixture was stirred at 50° C. overnight. After that time reaction mixture was cooled down to RT and NaBH₃CN (0.040 g, 0.638 mmol) was added. The solution was stirred at 50° C. for 1 h. The reaction mixture was cooled down to RT and quenched with 1M NaOH aq. solution, the product was extracted with AcOEt (×3) and all the combined organic layers were dried over Na₂SO₄, filtered and concentrated. The crude material was purified via FCC (from 100% DCM to 10% MeOH in DCM) then it was repurified again via preparative HPLC (ACN+0.1% NH₃, H₂O+0.1% NH₃) to give the desired product as a white solid (30 mg, 42%).

| | t_{R} | | |
|---|---|---|---|
| Ex. | (min) | Method | Analytical data NMR |
| Ex. 36 | 2.39 | 4 | ¹H NMR (300 MHz, Methanol-d4) δ 8.43 (s, |
| | | | 1H), 8.23 (t, J = 2.0 Hz, 1H), 8.16 (s, 2H), |
| | | | 8.16-8.14 (m, 1H), 8.13 (d, J = 1.5 Hz, 1H), |
| | | | 8.06 (s, 1H), 8.03 (d, J = 8.4 Hz, 1H), 7.81 |
| | | | (d, J = 8.1 Hz, 1H), 7.64 (s, 1H), 7.37 (t, |
| | | | J = 1.3 Hz, 1H), 7.17 (t, J = 54.7 Hz, 2H), 4.64 |
| | | | (s, 2H), 2.27 (d, J = 1.0 Hz, 3H). |

### Example 37

4-methyl-3-((pyrimidin-5-ylamino)methyl)-N-(3-(trifluoromethoxy) phenyl)benzamide

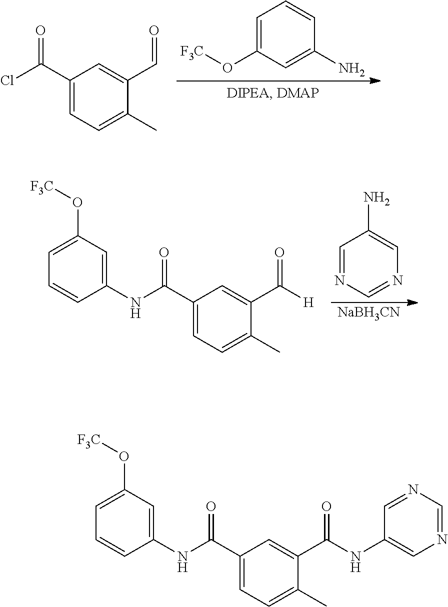

Step 1: Preparation of 3-formyl-4-methyl-N-(3-(trifluoromethoxy)phenyl) benzamide 3-formyl-4-methylbenzoyl chloride (0.6 g, 3.29 mmol) prepared as in Example 34, step 1, was dissolved in THF (3.22 ml) and this solution was added to a solution of 3-(trifluoro methoxy)aniline (0.582 g, 3.29 mmol), DIPEA (0.687 ml, 3.94 mmol) and DMAP (0.016 g, 0.131 mmol) in THF (6.44 ml). The reaction mixture was stirred at RT overnight. The reaction mixture was concentrated and the crude material was dissolved in saturated NaHCO₃ and extracted with DCM (×3). The all combined organic layers were washed with 5% citric acid, dried over Na₂SO₄, filtered and concentrated. The crude material was purified via FCC (from 100% Hexane to 30% AcOEt in Hexane) to give the desired product (450 mg, 42%).

¹H NMR (300 MHz, Chloroform-d) δ 10.37 (s, 1H), 8.29 (d, J=2.0 Hz, 1H), 8.06 (dd, J=8.0, 2.0 Hz, 1H), 8.01 (s, 1H), 7.73 (s, 1H), 7.56-7.50 (m, 1H), 7.46-7.35 (m, 2H), 7.07-7.00 (m, 1H), 2.76 (s, 3H).

Step 2: Preparation of 4-methyl-3-((pyrimidin-5-ylamino)methyl)-N-(3-(trifluoromethoxy)phenyl)benzamide

3-formyl-4-methyl-N-(3-(trifluoromethoxy)phenyl)benzamide (0.1 g, 0.309 mmol) and pyrimidin-5-amine (0.029 g, 0.309 mmol) were dissolved in MeOH (1.547 ml) and AcOH (0.053 ml, 0.928 mmol). The mixture was stirred at 50° C. for 1 h, then the reaction mixture was cooled down to RT and NaBH₃CN (0.087 g, 1.392 mmol) was added. The solution was stirred at 50° C. for 1 h. The reaction mixture was cooled down to RT and diluted with DCM. Solution was extracted with saturated NaHCO₃, aqueous layer was washed with DCM (×2). All organic phases were combined, dried over Na₂SO₄, filtered and concentrated. The crude material was purified via FCC (from 100% DCM to 5% MeOH in DCM) to give the desired product (63 mg, 51%).

| | t_{R} | | |
|---|---|---|---|
| Ex. | (min) | Method | Analytical data NMR |
| Ex. 37 | 2.75 | 6 | ¹H NMR (300 MHz, Methanol-d4) δ 8.40 (s, |
| | | | 1H), 8.17 (s, 2H), 7.94 (d, J = 1.9 Hz, 1H), |
| | | | 7.84-7.77 (m, 2H), 7.67-7.59 (m, 1H), 7.46- |
| | | | 7.36 (m, 2H), 7.07-6.99 (m, 1H), 4.43 (s, 2H), |
| | | | 2.48 (s, 3H). |

Pharmacological Activity of the Compounds of the Invention

In Vitro Assays

Binding Assays

DDR1 and DDR2 binding assays were performed using Life Technologies LanthaScreen™ Europium Kinase Binding assay. The compounds were incubated with 5 nM DDR1 (Carna Biosciences) or 5 nM DDR2 (Life Technologies) for 1 hour at room temperature in white 384-well OptiPlate (PerkinElmer), containing 20 nM or 10 nM Kinase Tracer 178 respectively and 2 nM Europium labelled anti-GST antibody (Life Technologies) in assay buffer (50 mM HEPES pH 7.5, 10 mM MgCl2, 1 mM EGTA and 0.01% BRIJ35).

The ratio of fluorescence emission 665 nm/615 nm after excitation at 340 nm was obtained using the Tecan Spark 20M plate reader. IC₅₀ values were determined in GraphPad Prism 7.0 software, using 4 parameter model: log(inhibitor) vs. response. IC₅₀ values were converted in Ki using the Cheng-Prusoff equation (Ki=IC₅₀/(1+[Tracer]/Kd).

DDR1 Cell Based Assay

The inhibition of DDR1 receptor activation by compounds was evaluated by PathHunter® U2OS DDR1 assay (Eurofins DiscoverX), according to the manufacturer's instructions. Briefly, U2OS-DDR1 cells were seeded in white 384-well plates at a density of 5000 cells/well and incubated for 2 hours at 37° C. and 5% CO₂. Cells were then treated with compounds at different concentrations and incubated for 30 minutes, before stimulation with bovine Type II Collagen 20 μg/ml and incubation overnight at 37° C. and 5% CO₂. PathHunter Detection Reagents were prepared according to the protocol provided by DiscoverX and 20 μl/well of this mix were added to each well. After incubating the plates for 1 hour at room temperature in the dark, luminescence signal was acquired with a plate reader. Raw data were normalized to vehicle control (0% for normalization) and positive control (100% for normalization; cells treated with 20 μg/ml collagen II) and IC₅₀ parameters were calculated in GraphPad Prism 8.0 software, using sigmoidal dose-response curve fitting with variable slope.

DDR2 Cell Based Assay

The inhibition of DDR2 phosphorylation by compounds was evaluated in HEK293T-DDR2 recombinant cells by phospho-ELISA assay. Briefly, HEK293T-DDR2 cells were seeded in poly-D-lysine-coated 24-well plates at a density of 250.000 cells/well and incubated for 1.5 hours at 37° C. and 5% CO₂ in DMEM+10% FBS. After that, the medium was changed to serum-free DMEM and cells were incubated for 3 hours. Then. test compounds were added at different concentrations 30 minutes before stimulation with bovine Type II Collagen at 50 μg/ml for further 3 hours. For DDR2 phospho-ELISA assay (DuoSet IC Human Phospho-DDR2; R&D Systems), protein extracts were obtained by adding 60 μl/well of lysis buffer prepared according to the manufacturer's instructions. Protein concentration in the samples was determined by BCA assay and the levels of phospho-DDR2 were determined following R&D Systems indications. Raw data were normalized to maximal inhibition control (0% for normalization) and positive control (100% for normalization; cells treated with 20 μg/ml collagen II) and IC₅₀ parameters were calculated in GraphPad Prism 8.0 software, using sigmoidal dose-response curve fitting with variable slope.

The results of the binding assay for individual compounds are provided below in Table 2 wherein the compounds are classified in term of potency with respect to their inhibitory activity expressed as Ki on DDR1 and DDR2:

TABLE 2

| Example No. | Ki DDR1 | Ki DDR2 |
|---|---|---|
| Example 22 | +++ | +++ |
| Example 19 | +++ | +++ |
| Example 23 | +++ | +++ |
| Example 24 | +++ | +++ |
| Example 25 | +++ | +++ |
| Example 26 | +++ | +++ |
| Example 21 | +++ | +++ |
| Example 20 | +++ | +++ |
| Example 1 | +++ | +++ |
| Example 18 | +++ | ++ |
| Example 28 | + | + |
| Example 30 | +++ | +++ |
| Example 29 | +++ | ++ |
| Example 10 | +++ | ++ |
| Example 27 | + | + |
| Example 9 | +++ | +++ |
| Example 16 | ++ | ++ |
| Example 17 | ++ | ++ |
| Example 11 | + | + |
| Example 12 | +++ | ++ |
| Example 14 | ++ | ++ |
| Example 15 | ++ | ++ |
| Example 2 | +++ | + |
| Example 6 | + | + |
| Example 5 | +++ | +++ |
| Example 4 | ++ | ++ |
| Example 8 | ++ | ++ |
| Example 7 | ++ | ++ |
| Example 3 | ++ | ++ |
| Example 13 | +++ | +++ |
| Example 31 | +++ | +++ |
| Example 32 | +++ | +++ |
| Example 33 | +++ | +++ |
| Example 34 | +++ | +++ |
| Example 35 | +++ | +++ |
| Example 36 | +++ | +++ |
| Example 37 | +++ | +++ |
| Example 38 | +++ | +++ |

DDR1
+: Ki comprised between 1000 and 300 nM
++: Ki comprised between 300 and 30 nM
+++: Ki lower than 30 nM
DDR2
Ki comprised between 1000 and 300 nM
++: Ki comprised between 300 and 30 nM
+++: Ki lower than 30 nM

In below Table 4 some compounds of the invention are classified in term of potency (IC₅₀) with respect to their inhibitory activity on DDR1 and DDR2 receptors, according to the cell based assay.

TABLE 4

| Example No. | IC₅₀ DDR1 | IC₅₀ DDR2 |
|---|---|---|
| EXAMPLE 20 | ++ | + |
| EXAMPLE 31 | ++ | ++ |
| EXAMPLE 32 | ++ | ++ |
| EXAMPLE 34 | ++ | ++ |
| EXAMPLE 37 | ++ | + |
| EXAMPLE 38 | ++ | + |
| EXAMPLE 35 | ++ | ++ |

DDR1
+: IC₅₀ comprised between 15 and 10 nM
++: IC₅₀ lower than 10 nM
DDR2
+: IC₅₀ comprised between 15 and 10 nM
++: IC₅₀ lower than 10 nM

As it can be appreciated, the compounds of Table 2 and 4, show a good activity as antagonists of DDR1 and DDR2 receptors. Accordingly, the compounds of the invention can be effectively used for treating disease, disorder or condition associated with DDR receptors, such as fibrosis, e.g. pulmonary fibrosis, idiopathic pulmonary fibrosis (IPF), hepatic fibrosis, renal fibrosis, ocular fibrosis, cardiac fibrosis, arterial fibrosis and systemic sclerosis.

1: A compound of formula (I) 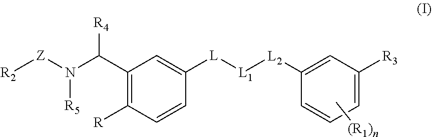
wherein
L and L₁ are different and independently selected from the group consisting of —C(O) and NH;
L₂ is absent;
Z is absent or selected from the group consisting of —CH₂ and —C(O);
R₁ is selected from the group consisting of —O(C₁-C₄)alkyl, 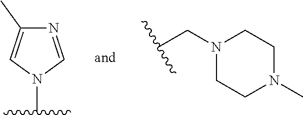
and is in a meta position with respect to the rest of the molecule;
n is 1;
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₄ is H;
R₅ is H or selected from the group consisting of (C₁-C₄)alkyl and heteroaryl(C₁-C₄)alkyl-;
R₆ is H or (C₁-C₄)alkyl;
or a pharmaceutically acceptable salt of said compound.

2. (canceled)

3: The compound or salt thereof according to claim 1, wherein L and L₁ are different and independently selected from the group consisting of —C(O) and NH;
Z is absent or selected from the group consisting of —CH₂ and —C(O);
R₁ is selected from the group consisting of —OCH₃, 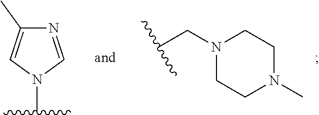
n is 1;
R is selected from the group consisting of methyl, ethyl, propyl and isopropyl;
R₂ is selected from the group consisting of pyrimidinyl, pyridinyl, imidazo[1,2-a]pyridinyl, 1H-pyrrolo[2,3-b]pyridinyl, pyrazolo[1,5-a]pyrimidinyl, 1H-indazolyl, indazolyl, 4,5,6,7-tetrahydropyrazolo[1,5-a]pyrimidinyl and benzo[d]thiazolyl wherein each of said heteroaryl and heterocycloalkyl may be optionally substituted by one or more —C(O)NHR₆ and CN;
R₃ is trifluoromethyl;
R₅ is H or selected from the group consisting of methyl, ethyl and 3-methylimidazo[1,2-a]pyridinyl; and
R₆ is H or methyl.

4: The compound or salt thereof according to claim 1, wherein R₁ is 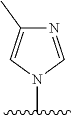 [SMILES: *n1cnc(C)c1]
and the compound is represented by the general formula (Ib) 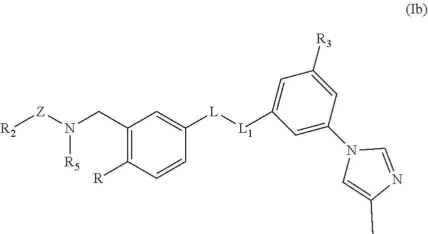
wherein
L and L₁ are different and independently selected from the group consisting of —C(O) and NH;
Z is absent or selected from the group consisting of —CH₂ and —C(O);
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-; and
R₆ is H or (C₁-C₄)alkyl.

5: The compound or salt thereof according to claim 1, wherein R₁ is 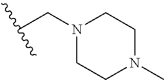 [SMILES: *CN1CCN(C)CC1]
and the compound is represented by the general formula (Ic) 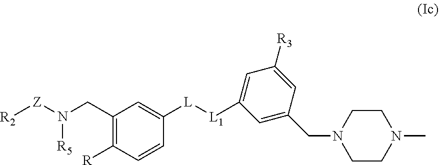
wherein
L and L₁ are different and independently selected from the group consisting of —C(O) and NH;
Z is absent or selected from the group consisting of —CH₂ and —C(O);
R is (C₁-C₄)alkyl;
R₂ is selected from the group consisting of heteroaryl and heterocycloalkyl wherein each of said heteroaryl and heterocycloalkyl may be optionally substituted by one or more —C(O)NHR₆ and CN;
R₃ is (C₁-C₄)haloalkyl;
R₅ is H or selected from the group consisting of (C₁-C₄)alkyl and heteroaryl(C₁-C₄)alkyl-; and
R₆ is H or (C₁-C₄)alkyl.

6: The compound or salt thereof according to claim 1, wherein L₂ is absent, R₄ and R₅ are —H, Z is absent, and the compound is represented by the general formula (If) 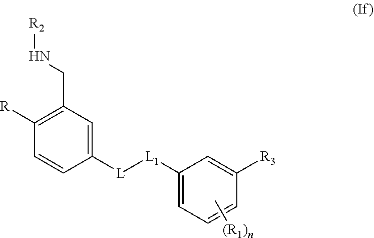
wherein
L is —C(O); L₁ is —NH;
R₁ is selected from the group consisting of —O(C₁-C₄)alkyl and 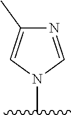 [SMILES: *n1cnc(C)c1]
and is in a meta position with respect to the rest of the molecule;
R is (C₁-C₄)alkyl;
R₂ is selected from the group consisting of 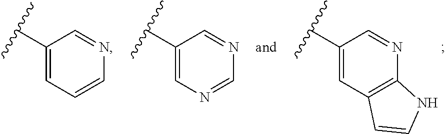
and
R₃ is (C₁-C₄)haloalkyl.

7: The compound or salt thereof according to claim 6, wherein L is —C(O); L₁ is —NH; R₁ is selected from the group consisting of —OCH₃ and 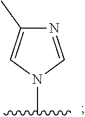 [SMILES: *n1cnc(C)c1]
R is methyl;
R₂ is selected from the group consisting of 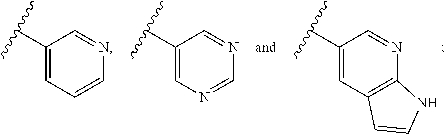
and
R₃ is trifluoromethyl.

8: The compound or salt thereof according to claim 7, wherein L is —C(O), L₁ is —NH, R₁ is OCH₃, R is selected from the group consisting of methyl and fluorine, R₂ is selected from the group consisting of 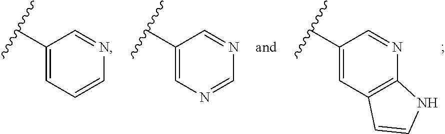
and
R₃ is trifluoromethyl.

9: The compound or salt thereof according to claim 1, wherein the compound is selected from the group consisting of:
N-(2-methyl-5-((3-(4-methyl-1H-imidazol-1-yl)-5-(trifluoromethyl)phenyl)carbamoyl)benzyl)imidazo[1,2-a]pyridine-3-carboxamide;
4-methyl-N-(3-((4-methylpiperazin-1-yl)methyl)-5-(trifluoromethyl)phenyl)-3-((pyrimidin-5-ylamino)methyl)benzamide;
N-methyl-4-((2-methyl-5-((3-(4-methyl-1H-imidazol-1-yl)-5-(trifluoromethyl)phenyl)carbamoyl)benzyl)amino)picolinamide;
N-methyl-4-((2-methyl-5-((3-((4-methylpiperazin-1-yl)methyl)-5-(trifluoromethyl)phenyl)carbamoyl)benzyl)amino)picolinamide;
N-(2-methyl-5-((3-(4-methyl-1H-imidazol-1-yl)-5-(trifluoromethyl)phenyl)carbamoyl)benzyl)-1H-pyrrolo[2,3-b]pyridine-5-carboxamide;
N-(2-methyl-5-((3-((4-methylpiperazin-1-yl)methyl)-5-(trifluoromethyl)phenyl)carbamoyl)benzyl)-1H-pyrrolo[2,3-b]pyridine-5-carboxamide;
N-(2-methyl-5-((3-((4-methylpiperazin-1-yl)methyl)-5-(trifluoromethyl)phenyl)carbamoyl)benzyl)imidazo[1,2-a]pyridine-3-carboxamide
4-methyl-N-(3-(4-methyl-1H-imidazol-1-yl)-5-(trifluoromethyl)phenyl)-3-((pyrimidin-5-ylamino)methyl)benzamide;
4-methyl-N-(3-(4-methyl-1H-imidazol-1-yl)-5-(trifluoromethyl)phenyl)-3-((pyrazolo[1,5-a]pyrimidin-6-ylamino)methyl)benzamide;
4-methyl-N-(3-((4-methylpiperazin-1-yl)methyl)-5-(trifluoromethyl)phenyl)-3-((pyrazolo[1,5-a]pyrimidin-6-ylamino)methyl)benzamide;
N-(5-((3-((4-methylpiperazin-1-yl)methyl)-5-(trifluoromethyl)phenyl)carbamoyl)-2-propylbenzyl)imidazo[1,2-a]pyridine-3-carboxamide formate salt;
4-isopropyl-N-(3-((4-methylpiperazin-1-yl)methyl)-5-(trifluoromethyl)phenyl)-3-((pyrimidin-5-ylamino)methyl)benzamide;
N-(3-((4-methylpiperazin-1-yl)methyl)-5-(trifluoromethyl)phenyl)-4-propyl-3-((pyrimidin-5-ylamino)methyl)benzamide;
N-methyl-4-((2-methyl-5-(3-((4-methylpiperazin-1-yl)methyl)-5-(trifluoromethyl)benzamido)benzyl)amino)picolinamide;
N-(2-isopropyl-5-((3-((4-methylpiperazin-1-yl)methyl)-5-(trifluoromethyl)phenyl)carbamoyl)benzyl)-1H-pyrrolo[2,3-b]pyridine-5-carboxamide;
N-(4-methyl-3-((pyrimidin-5-ylamino)methyl)phenyl)-3-((4-methylpiperazin-1-yl)methyl)-5-(trifluoromethyl)benzamide;
4-isopropyl-N-(3-((4-methylpiperazin-1-yl)methyl)-5 (trifluoromethyl)phenyl)-3-((pyrazolo[1,5-a]pyrimidin-6 ylamino)methyl)benzamide;
N-(3-((4-methylpiperazin-1-yl)methyl)-5-(trifluoromethyl)phenyl)-4-propyl-3-((pyrazolo[1,5-a]pyrimidin-6-ylamino)methyl)benzamide;
N-(4-methyl-3-(((pyrimidin-5-ylmethyl)amino)methyl)phenyl)-3-((4-methylpiperazin-1-yl)methyl)-5-(trifluoromethyl)benzamide;
N-(3-((bis(imidazo[1,2-a]pyridin-3-ylmethyl)amino)methyl)-4-methylphenyl)-3-((4-methylpiperazin-1-yl)methyl)-5-(trifluoromethyl)benzamide;
N-methyl-4-(((2-methyl-5-(3-((4-methylpiperazin-1-yl)methyl)-5-(trifluoromethyl)benzamido)benzyl)amino)methyl)picolinamide;
N-(3-((((1H-pyrrolo[2,3-b]pyridin-5-yl)methyl)amino)methyl)-4-methylphenyl)-3-((4-methylpiperazin-1-yl)methyl)-5-(trifluoromethyl)benzamide;
3-((ethyl(pyrazolo[1,5-a]pyrimidin-6-yl)amino)methyl)-4-methyl-N-(3-(4-methyl-1H-imidazol-1-yl)-5-(trifluoromethyl)phenyl)benzamide;
4-methyl-N-(3-(4-methyl-1H-imidazol-1-yl)-5-(trifluoromethyl)phenyl)-3-(((4,5,6,7-tetrahydropyrazolo[1,5-a]pyrimidin-6-yl)amino)methyl)benzamide;
3-(((1H-pyrrolo[2,3-b]pyridin-5-yl)amino)methyl)-4-methyl-N-(3-(4-methyl-1H-imidazol-1-yl)-5-(trifluoromethyl)phenyl)benzamide;
3-(((1H-indazol-5-yl)amino)methyl)-4-methyl-N-(3-(4-methyl-1H-imidazol-1-yl)-5-(trifluoromethyl)phenyl)benzamide;
3-(((5-cyanopyridin-2-yl)amino)methyl)-4-methyl-N-(3-(4-methyl-1H-imidazol-1-yl)-5-(trifluoromethyl)phenyl)benzamide;
3-(((2-cyanopyridin-4-yl)amino)methyl)-4-methyl-N-(3-(4-methyl-1H-imidazol-1-yl)-5-(trifluoromethyl)phenyl)benzamide;
3-((benzo[d]thiazol-6-ylamino)methyl)-4-methyl-N-(3-(4-methyl-1H-imidazol-1-yl)-5-(trifluoromethyl)phenyl)benzamide;
N-(3-(((imidazo[1,2-a]pyridin-3-ylmethyl)amino)methyl)-4-methylphenyl)-3-((4-methylpiperazin-1-yl)methyl)-5-(trifluoromethyl)benzamide; and
N-(4-methoxy-3-(trifluoromethyl)phenyl)-4-methyl-3-((pyrimidin-5-ylamino)methyl)benzamide.

10: A pharmaceutical composition comprising the compound or salt thereof of formula (I) according to claim 1, in admixture with one or more pharmaceutically acceptable carriers or excipients.

11-12. (canceled)

13: A method of treating a disease, disorder, or condition associated with dysregulation of DDR1 and DDR2, comprising administering the compound or salt thereof according to claim 1 to a patient in need thereof.

14: A method for treating fibrosis and/or diseases, disorders, or conditions that involve fibrosis, comprising administering the compound or salt thereof according to claim 1 to a patient in need thereof.

15: The method according to claim 14, wherein the fibrosis is at least one selected from the group consisting of pulmonary fibrosis, idiopathic pulmonary fibrosis (IPF), hepatic fibrosis, renal fibrosis, ocular fibrosis, cardiac fibrosis, arterial fibrosis and systemic sclerosis.

16: The method according to claim 15, wherein the fibrosis comprises idiopathic pulmonary fibrosis (IPF).

17: A compound selected from the group consisting of:
4-methyl-3-((pyrimidin-5-ylamino)methyl)-N-(3-(trifluoromethyl)phenyl)benzamide;
3-(((1H-pyrrolo[2,3-b]pyridin-5-yl)amino) methyl)-4-fluoro-N-(3 (trifluoromethyl)phenyl) benzamide;
4-methyl-3-((pyridin-3-ylamino)methyl)-N-(3-(trifluoromethyl)phenyl)benzamide;
4-fluoro-3-(((5-(1-methyl-1H-pyrazol-3-yl)pyridin-3-yl)amino)methyl)-N-(3-(trifluoromethoxy)phenyl)benzamide;
4-(difluoromethyl)-N-(3-(4-methyl-1H-imidazol-1-yl)-5-(trifluoromethyl)phenyl)-3-((pyrimidin-5-yl amino)methyl)benzamide;
4-methyl-3-((pyrimidin-5-ylamino)methyl)-N-(3-(trifluoromethoxy)phenyl)benzamide; and
3-(((1H-pyrrolo[2,3-b]pyridin-5-yl)amino)methyl)-4-fluoro-N-(3-(trifluoromethoxy)phenyl)benzamide.

18: A pharmaceutical composition comprising the compound or salt thereof according to claim 17, in admixture with one or more pharmaceutically acceptable carriers or excipients.

19: A method for treating fibrosis and/or diseases, disorders, or conditions that involve fibrosis, comprising administering the compound or salt thereof according to claim 17 to a patient in need thereof.

20: The method according to claim 19, wherein the fibrosis comprises idiopathic pulmonary fibrosis (IPF).
